(19) United States
(12) Patent Application Publication
(10) Pub. No.: US 2023/0416758 A1
(43) Pub. Date: Dec. 28, 2023

(54) TRNA FRAGMENTS AND METHODS OF USE

(71) Applicant: Thomas Jefferson University, Philadelphia, PA (US)

(72) Inventors: Yohei Kirino, Philadelphia, PA (US); Kamlesh Ganesh Pawar, Philadelphia, PA (US)

(21) Appl. No.: 18/254,027

(22) PCT Filed: Nov. 23, 2021
(86) PCT No.: PCT/US2021/060430

§ 371 Date: May 23, 2023

Related U.S. Application Data
(60) Provisional application No. 63/117,167, filed on Nov. 23, 2020.

(51) Int. Cl. C12N 15/117; A61P 37/02

(52) U.S. Cl. CPC C12N 15/117; A61P 37/02; C12N 2310/17

(57) ABSTRACT

The present invention provides fragments of tRNA molecules and methods of use thereof to modulate toll like receptor (TLR) signaling, for immunotherapy and for other therapeutic applications.

### CROSS REFERENCE TO RELATED APPLICATIONS

This application claims priority to U.S. Provisional Application No. 63/117,167, filed Nov. 23, 2020 which is hereby incorporated by reference herein in its entirety.

### STATEMENT REGARDING FEDERALLY SPONSORED RESEARCH OR DEVELOPMENT

This invention was made with government support under Grant No. RO1 GM106047, R21 AI130496, and RO1 HL150560 awarded by the National Institute of Health. The government has certain rights in the invention.

### BACKGROUND OF THE INVENTION

There are many pathogenic microbes that induce a wide range of symptoms and diseases, including Mycobacterium tuberculosis (Mtb), one of the greatest threats to humans, causing more than 1.2 million deaths annually (WHO, Global Tuberculosis Report, 2020). When a host is infected with pathogenic microbes, it has two essential arms of defense to eliminate them: the innate immune system and the adaptive immune system (Parkin J, et al., 2001, Lancet, 357: 1777-1789). In the innate immune system, Toll-like receptors (TLRs) and other pathogen recognition receptors detect pathogen-associated molecular patterns (PAMPs) and initiate protective responses (Brubaker S W, et al., 2015, Annu Rev Immunol, 33: 257-290; Kawai T, et al., 2010, Nat Immunol, 11: 373-384). Among the 10 TLRs characterized in humans, TLR1, -2, -4, -5, -6, and -10 localize to the cell surface (surface TLRs), while TLR3, -7, -8, and -9 localize to intracellular compartments such as endosomes (endosomal TLRs). When TLRs recognize PAMPs, they recruit adaptor proteins, such as MyD88 and TRIF, to initiate signal transduction pathways that culminate in the activation of transcription factors such as NF-κB and AP-1, leading to the production of cytokines and chemokines for host defense (Kawasaki T, et al., 2014, Front Immunol, 5: 461; Satoh, T, et al., 2017, Myeloid Cells in Health and Disease: A Synthesis, 447-453).

Endosomal TLRs are known to sense nucleic acids, which act as ligands (Heil F, et al., 2004, Science. 303: 1526-1529; Zhang Z, et al., 2016, Immunity, 45: 737-748). Of the endosomal TLRs, TLR7 and -8 recognize single-stranded RNAs (ssRNAs), whereas TLR3 and -9 recognize dsRNAs and ssDNAs, respectively. TLR7 and -8 are primarily expressed in immune cells such as monocytes/macrophages, dendritic cells, neutrophils, and B cells, and their recognition of pathogen-derived ssRNAs (e.g., viral and bacterial ssRNAs) recruits MyD88, activates NF-κB-mediated transcription, and induces the production of interferons and cytokines (Blasius A L, et al., 2010, Immunity 32: 305-315). Besides pathogen-derived ssRNAs, TLR7 and -8 also sense host ssRNAs, such as microRNAs (miRNAs). miRNAs can be incorporated into extracellular vehicles (EVs), and those EV-miRNAs can reach and function as agonist of endosomal TLR7 and -8 in recipient cells (Lehmann S M, et al., 2012, Nat Neurosci, 15: 827-835; Fabbri M, et al., 2012, Proc Natl Acad Sci USA, 109: E2110-2116; Temoche-Diaz, M M, et al., 2019, Elife, 8: e47544). The activation of TLR7 and -8 by miRNAs is involved not only in the immune response (Ranganathan P, et al., 2017, J Immunol, 198: 2500-2512; Feng Y, et al., 2017, J Immunol, 199: 2106-2117), but also in tumor growth and metastasis (Alexander M, et al., 2015, Nat Commun, 6(1), 1-16; Casadei L, et al., Cancer Res, 77: 3846-3856; Fabbri M, 2012, Cancer Res, 72: 6333-6337) and in neuronal damage and apoptosis (Lehmann S M, et al., 2012, Nat Neurosci, 15: 827-835; Coleman L G, et al., 2017, J Neuroinflammation, 14: 22). Considering that EV contains many other RNA species (e.g., messenger RNAs [mRNAs], transfer tRNAs [tRNAs], small nucleolar RNAs [snoRNAs], Y-RNAs, vault RNAs, and long non-coding RNAs [lncRNAs]) (Shurtleff M J, et al., 2017, Proc Natl Acad Sci USA, 114: E8987-E8995; Nolte-'t Hoen E N, et al., 2012, Nucleic Acids Res, 40: 9272-9285), it is not surprising that those EV-RNAs are also deliverable to endosomal TLRs and function as their ligands, though this possibility remains unexplored.

Although tRNAs are best known as essential adapter molecules of translational machinery, recent studies have established their role as a source of short non-coding RNAs (ncRNAs) (Sobala A, et al., 2011, Wiley Interdiscip Rev RNA, 2: 853-862; Anderson P, et al., 2014, FEBS Lett, 588: 4297-4304; Kumar P, et al., 2016, Trends Biochem Sci, 41: 679-689; Shigematsu M, et al., 2015, Gene Regul Syst Bio, 9: 27-33). In many organisms, specific tRNA-derived ncRNAs are expressed as functional molecules and are involved in various biological processes beyond translation. tRNA-derived ncRNAs can be classified into two groups: tRNA halves and shorter tRNA-derived fragments (tRFs). Among them, 5′-tRNA halves, which comprise the region from the 5′-end to the anticodon-loop of tRNAs, are one of the most abundant classes. In mammalian cells, they are generated from angiogenin (ANG)-mediated anticodon cleavage of tRNAs (Fu H, et al., 2009, FEBS Lett, 583: 437-442; Yamasaki S, et al., 2009, J Cell Biol, 185: 35-42) and have been shown to regulate translation, promote stress response, promote cell proliferation, and be associated with cancers, neurodegenerative diseases, and metabolic disorders (Sobala A, et al., 2011, Wiley Interdiscip Rev RNA, 2: 853-862; Anderson P, et al., 2014, FEBS Lett, 588: 4297-4304; Kumar P, et al., 2016, Trends Biochem Sci, 41: 679-689; Shigematsu M, et al., 2015, Gene Regul Syst Bio, 9: 27-33; Ivanov P, et al., 2014, Proc Natl Acad Sci USA, 111: 18201-18206; Honda S, et al., 2015, Proc Natl Acad Sci USA, 112: E3816-3825; Zhang Y, et al., 2018, Nat Cell Biol, 20: 535-540). 5′-tRNA halves further function as direct precursors of Piwi-interacting RNAs (piRNAs) in germ cells (Honda S, et al., 2017, Nucleic Acids Res, 45: 9108-9120).

Despite their demonstrated functionality, information regarding the expression profiles of 5′-tRNA halves and their regulation remains elusive, in part because 5′-tRNA halves are not captured by standard RNA sequencing (RNA-seq). As a result of ANG-catalyzed biogenesis, 5′-tRNA halves contain a 2′,3′-cyclic phosphate (cP) at their 3′-end (Honda S, et al., 2015, Proc Natl Acad Sci USA, 112: E3816-3825; Shigematsu M, et al., 2018, Front Genet, 9: 562). These cP-containing RNAs (cP-RNAs) are not ligated to a 3′-adapter during cDNA amplification, and thus they are not amplified in standard RNA-seq procedures. This limitation remains for cP-RNAs, including 5′-tRNA halves, to form uncharacterized components in the transcriptomes (Shigematsu M, et al., 2018, Front Genet, 9: 562). To resolve this issue, “cP-RNA-seq” was developed (Honda S, et al., 2015, Proc Natl Acad Sci USA, 112: E3816-3825; Honda S, et al., 2016, Nat Protoc, 11: 476-489), which is able to specifically sequence cP-RNAs, and subsequently used to identify a comprehensive expression repertoire of 5′-tRNA halves and other cP-RNA species in human and Bombyx cultured cells and mouse tissues (Honda S, et al., 2015, Proc Natl Acad Sci USA, 112: E3816-3825; Honda S, et al., 2017, Nucleic Acids Res, 45: 9108-9120; Shigematsu M, et al., 2020, RNA Biol, 17: 1060-1069; Shigematsu M, et al., 2019, PLoS Genet, 15: e1008469).

Although endosomal TLR7 recognizes single-stranded RNAs as its ligands, as described above, their endogenous RNA ligands have not been fully explored. TLR7 agonists can be used for immunotherapy, adjuvant strategy, antiviral/antibacterial action, and treatments of allergy and asthma. Pharmaceutical companies and research institutions are thereby trying to develop synthetic compounds which work as TLR7 modulators (such as derivatives of imidazoquinolines). However, those synthetic compounds show toxicity. The natural ligands for TLR7, namely 5′-tRNA half molecules, could function as superior TLR7 modulators with lower cellular toxicity.

There remains a need in the art for improved compositions and methods for boosting immunity. This invention satisfies this unmet need.

### SUMMARY OF THE INVENTION

In one embodiment, the invention relates to a method of treating or preventing a disease or disorder in a subject in need thereof, the method comprising administering a nucleic acid molecule comprising a fragment or variant of a tRNA molecule to the subject.

In one embodiment, the fragment or variant of a tRNA molecule activates at least one toll-like receptor (TLR). In one embodiment, the TLR is TLR7, TLR8 or a combination thereof.

In one embodiment, the fragment or variant of a tRNA molecule comprises a fragment comprises at least 4 nucleotides of a tRNA molecule. In one embodiment, the tRNA is tRNA^{HisGUG}, tRNA^{GluCUC}, tRNA^{ValCAC}, tRNA^{GlyGCC}, tRNA^{ValAAC}, tRNA^{GluUUC}, tRNA^{LysCUU}, tRNA^{AspGUC}, tRNA^{MetCAU}, tRNA^{ProAGG}, tRNA^{LeuCAG}, tRNA^{ArgUCU}, tRNA^{LysUUU}, tRNA^{ValUAC}, tRNA^{GlnCUG}, tRNA^{ArgCCG}, tRNA^{ArgACG}, tRNA^{LeuUAA}, tRNA^{ArgUCG}, tRNA^{AsnGUU}, tRNA^{AlaCGC}, tRNA^{LeuAAG}, tRNA^{ThrUGU}, tRNA^{AlaAGC}, tRNA^{LeuCAA}, tRNA^{PheGAA}, tRNA^{GlnUUG}, tRNA^{TrpCCA}, tRNA^{SerGCU}, tRNA^{CysGCA}, tRNA^{IleAAU}, tRNA^{ArgCCU}, tRNA^{SerAGA}, tRNA^{GlyUCC}, tRNA^{LeuUAG}, tRNA^{AlaUGC}, or tRNA^{ThrCGU}. In one embodiment, the tRNA is tRNA^{HisGUG}, tRNA^{ValCAC}, or tRNA^{ValAAC}.

In one embodiment, the fragment or variant of a tRNA molecule comprises an RNA molecule comprising a nucleotide sequence of SEQ ID NO:1, SEQ ID NO:3, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91 or SEQ ID NO:92.

In one embodiment, the fragment or variant of a tRNA molecule comprises an RNA molecule comprising a fragment of a nucleotide sequence of SEQ ID NO:1, SEQ ID NO:3, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO: 76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91 or SEQ ID NO:92 comprising at least 4 nucleotides.

In one embodiment, the fragment or variant of a tRNA molecule comprises an RNA molecule comprising a variant of a nucleotide sequence of SEQ ID NO:1, SEQ ID NO:3, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91 or SEQ ID NO:92 comprising at least one modified nucleotide.

In one embodiment, the fragment or variant of a tRNA molecule comprises an RNA molecule comprising a variant of a nucleotide sequence of SEQ ID NO:1, SEQ ID NO:3, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91 or SEQ ID NO:92 comprising a sequence having at least 90% identity to an RNA molecule comprising a nucleotide sequence of SEQ ID NO:1, SEQ ID NO:3, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO: 67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO: 77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91 or SEQ ID NO:92. In one embodiment, the variant of SEQ ID NO:1 comprises SEQ ID NO:2.

In one embodiment, the disease or disorder is cancer or an infectious disease.

In one embodiment, the invention relates to a nucleic acid molecule comprising a fragment or variant of a tRNA molecule.

In one embodiment, the fragment or variant of a tRNA molecule activates at least one toll-like receptor (TLR). In one embodiment, the TLR is TLR7, TLR8 or a combination thereof.

In one embodiment, the fragment or variant of a tRNA molecule comprises a fragment comprises at least 4 nucleotides of a tRNA molecule. In one embodiment, the tRNA is tRNA^{HisGUG}, tRNA^{GluCUC}, tRNA^{ValCAC}, tRNA^{GlyGCC}, tRNA^{ValAAC}, tRNA^{GluUUC}, tRNA^{LysCUU}, tRNA^{AspGUC}, tRNA^{MetCAU}, tRNA^{ProAGG}, tRNA^{LeuCAG}, tRNA^{ArgUCU}, tRNA^{LysUUU}, tRNA^{ValUAC}, tRNA^{GlnCUG}, tRNA^{ArgCCG}, tRNA^{ArgACG}, tRNA^{LeuUAA}, tRNA^{ArgUCG}, tRNA^{AsnGUU}, tRNA^{AlaCGC}, tRNA^{LeuAAG}, tRNA^{ThrUGU}, tRNA^{AlaAGC}, tRNA^{LeuCAA}, tRNA^{PheGAA}, tRNA^{GlnUUG}, tRNA^{TrpCCA}, tRNA^{SerGCU}, tRNA^{CysGCA}, tRNA^{IleAAU}, tRNA^{ArgCCU}, tRNA^{SerAGA}, tRNA^{GlyUCC}, tRNA^{LeuUAG}, tRNA^{AlaUGC}, or tRNA^{ThrCGU}. In one embodiment, the tRNA is tRNA^{HisGUG}, tRNA^{ValCAC}, or tRNA^{ValAAC}.

In one embodiment, the fragment or variant of a tRNA molecule comprises an RNA molecule comprising a nucleotide sequence of SEQ ID NO:1, SEQ ID NO:3, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO: 77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91 or SEQ ID NO:92.

In one embodiment, the fragment or variant of a tRNA molecule comprises an RNA molecule comprising a fragment of a nucleotide sequence of SEQ ID NO:1, SEQ ID NO:3, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO: 76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91 or SEQ ID NO:92 comprising at least 4 nucleotides.

In one embodiment, the fragment or variant of a tRNA molecule comprises an RNA molecule comprising a variant of a nucleotide sequence of SEQ ID NO:1, SEQ ID NO:3, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO: 76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91 or SEQ ID NO:92 comprising at least one modified nucleotide.

In one embodiment, the fragment or variant of a tRNA molecule comprises an RNA molecule comprising a variant of a nucleotide sequence of SEQ ID NO:1, SEQ ID NO:3, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91 or SEQ ID NO:92 comprising a sequence having at least 90% identity to an RNA molecule comprising a nucleotide sequence of SEQ ID NO:1, SEQ ID NO:3, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO: 67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO: 77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91 or SEQ ID NO:92. In one embodiment, the variant of SEQ ID NO:1 comprises SEQ ID NO:2.

In one embodiment, the invention relates to a composition comprising a nucleic acid molecule comprising a fragment or variant of a tRNA molecule. In one embodiment, the fragment or variant of a tRNA molecule activates at least one toll-like receptor (TLR). In one embodiment, the TLR is TLR7, TLR8 or a combination thereof.

In one embodiment, the fragment or variant of a tRNA molecule comprises a fragment comprises at least 4 nucleotides of a tRNA molecule. In one embodiment, the tRNA is tRNA^{HisGUG}, tRNA^{GluCUC}, tRNA^{ValCAC}, tRNA^{GlYGCc}, tRNA^{ValAAC}, tRNA^{GluUUC}, tRNA^{LysCUU}, tRNA^{AspGUC}, tRNA^{MetCAU}, tRNA^{ProAGG}, tRNA^{LeuCAG}, tRNA^{ArgUCU}, tRNA^{LysUUU}, tRNA^{ValUAC}, tRNA^{GlnCUG}, tRNA^{ArgCCG}, tRNA^{ArgACG}, tRNA^{LeuUAA}, tRNA^{ArgUCG}, tRNA^{AsnGUU}, tRNA^{AlaCGC}, tRNA^{LeuAAG}, tRNA^{ThrUGU}, tRNA^{AlaAGC}, tRNA^{LeuCAA}, tRNA^{PheGAA}, tRNA^{GlnUUG}, tRNA^{TrpCCA}, tRNA^{SerGCU}, tRNA^{CysGCA}, tRNA^{IleAAU}, tRNA^{ArgCCU}, tRNA^{SerAGA}, tRNA^{GlyUCC}, tRNA^{LeuUAG}, tRNA^{AlaUGC}, or tRNA^{ThrCGU}. In one embodiment, the tRNA is tRNA^{HisGUG}, tRNA^{ValCAC}, or tRNA^{ValAAC}.

In one embodiment, the fragment or variant of a tRNA molecule comprises an RNA molecule comprising a nucleotide sequence of SEQ ID NO:1, SEQ ID NO:3, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO: 77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91 or SEQ ID NO:92.

In one embodiment, the fragment or variant of a tRNA molecule comprises an RNA molecule comprising a fragment of a nucleotide sequence of SEQ ID NO:1, SEQ ID NO:3, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91 or SEQ ID NO:92 comprising at least 4 nucleotides.

In one embodiment, the fragment or variant of a tRNA molecule comprises an RNA molecule comprising a variant of a nucleotide sequence of SEQ ID NO:1, SEQ ID NO:3, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO: 76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91 or SEQ ID NO:92 comprising at least one modified nucleotide.

In one embodiment, the fragment or variant of a tRNA molecule comprises an RNA molecule comprising a variant of a nucleotide sequence of SEQ ID NO:1, SEQ ID NO:3, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO: 76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91 or SEQ ID NO:92 comprising a sequence having at least 90% identity to an RNA molecule comprising a nucleotide sequence of SEQ ID NO:1, SEQ ID NO:3, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO: 77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91 or SEQ ID NO:92. In one embodiment, the variant of SEQ ID NO:1 comprises SEQ ID NO:2.

In one embodiment, the composition comprises a pharmaceutically acceptable excipient, an adjuvant, or a combination thereof.

In one embodiment, the composition further comprises at least one additional therapeutic agent.

In one embodiment, the additional therapeutic agent is an altered T-cell, a chimeric antigen receptor T-cell (CAR-T), an antigen, a vaccine, an antibody, an immune checkpoint inhibitor, a small molecule, a chemotherapeutic agent, or a stem cell.

In one embodiment, the invention relates to a method of increasing an immune response in a subject in need thereof, the method comprising administering a nucleic acid molecule comprising a fragment or variant of a tRNA molecule.

In one embodiment, the fragment or variant of a tRNA molecule activates at least one toll-like receptor (TLR). In one embodiment, the TLR is TLR7, TLR8 or a combination thereof.

In one embodiment, the fragment or variant of a tRNA molecule comprises a fragment comprises at least 4 nucleotides of a tRNA molecule. In one embodiment, the tRNA is tRNA^{HisGUG}, tRNA^{GluCUC}, tRNA^{ValCAC}, tRNA^{GlyGCC}, tRNA^{ValAAC}, tRNA^{GluUUC}, tRNA^{LysCUU}, tRNA^{AspGUC}, tRNA^{MetCAU}, tRNA^{ProAGG}, tRNA^{LeuCAG}, tRNA^{ArgUCU}, tRNA^{LysUUU}, tRNA^{ValUAC}, tRNA^{GlnCUG}, tRNA^{ArgCCG}, tRNA^{ArgACG}, tRNA^{LeuUAA}, tRNA^{ArgUCG}, tRNA^{AsnGUU}, tRNA^{AlaCGC}, tRNA^{LeuAAG}, tRNA^{ThrUGU}, tRNA^{AlaAGC}, tRNA^{LeuCAA}, tRNA^{PheGAA}, tRNA^{GlnUUG}, tRNA^{TrpCCA}, tRNA^{SerGCU}, tRNA^{CysGCA}, tRNA^{IleAAU}, tRNA^{ArgCCU}, tRNA^{SerAGA}, tRNA^{GlyUCC}, tRNA^{LeuUAG}, tRNA^{AlaUGC}, or tRNA^{ThrCGU}. In one embodiment, the tRNA is tRNA^{HisGUG}, tRNA^{ValCAC}, or tRNA^{ValAAC}.

In one embodiment, the invention relates to a method of activating a TLR in a subject in need thereof, the method comprising administering a nucleic acid molecule comprising a fragment or variant of a tRNA molecule or a composition comprising a nucleic acid molecule comprising a fragment or variant of a tRNA molecule to the subject. In one embodiment, the TLR is TLR7, TLR8 or a combination thereof.

### BRIEF DESCRIPTION OF THE DRAWINGS

The following detailed description of embodiments of the invention will be better understood when read in conjunction with the appended drawings. It should be understood that the invention is not limited to the precise arrangements and instrumentalities of the embodiments shown in the drawings.

FIG. 1 depicts exemplary read numbers of cP-RNA-seq sequence libraries. The sequence libraries contain ˜35-44 million raw reads and are publicly available from the NCBI Sequence Read Archive (accession No. SRR8430192, SRR8430191, and SRR8430193).

FIG. 2 depicts exemplary results of experiments demonstrating synthetic RNAs that were synthesized by in vitro transcription, gel-purified, and analyzed with denaturing PAGE.

FIG. 3, comprising FIGS. 3A-F, depicts exemplary results of experiments demonstrating upregulation of the expression of 5′-tRNA halves by Mycobacterium bovis bacillus Calmette-Guerin (BCG) infection and surface TLR activation. FIG. 3A depicts results from experiments showing total RNAs from HMDMs infected with viable or heat-killed (HK) BCG for 0.5 or 4 hours that were subjected to TaqMan RT-qPCR for 5′-tRNA^{HisGUG} half (5′-HisGUG) and 5′-tRNA^{GluCUC} half (5′-GluCUC). Non-infected HMDMs served as a control. The quantified 5′-tRNA half levels were normalized to U6 snRNA levels. Averages of three experiments with SD values are shown (*P<0.05, **P<0.01, and ***P<0.001; two-tailed t-Test). FIGS. 3B and 3C depict results of experiments showing total RNAs from HMDMs treated with LPS or PGN for 12 hours that were subjected to RT-qPCR for the indicated mRNAs (FIG. 3B) and to TaqMan RT-qPCR for the 5′-tRNA halves (FIG. 3C). HMDMs without treatment served as a control. The quantified 5′-tRNA half levels were normalized to the levels of U6 snRNA and GAPDH mRNA, respectively. FIG. 3D depicts results from experiments showing total RNAs from HMDMs treated with LPS or PGN that were subjected to northern blot for the 5′-tRNA halves and their corresponding mature tRNAs. miR-16 was analyzed as a control. FIGS. 3E and 3F depict results from experiments showing total RNAs from PHMDMs treated with LPS or PGN that were subjected to RT-qPCR for the indicated mRNAs (FIG. 3E) and to TaqMan RT-qPCR for the 5′-tRNA halves (FIG. 3F). PHMDMs without treatment served as a control.

FIG. 4, comprising FIGS. 4A-G, depicts exemplary results of experiments demonstrating NF-κB-mediated upregulation of the expression of ANG mRNA upon surface TLR activation. FIG. 4A depicts results of HMDMs that were transfected with control siRNA (siControl) or siRNA targeting ANG (siANG) and incubated for 60 h. LPS was then added and the cells were further cultured for 12 h. RT-qPCR confirmed the reduction of ANG mRNA upon siANG transfection (RPLP0: control). Averages of three experiments with SD values are shown (*P<0.05, **P<0.01, and ***P<0.001; two-tailed t-Test). FIG. 4B depicts results showing, after siRNA transfection and LPS treatment of HMDMs, RNAs isolated from the HMDMs that were subjected to quantification of 5′-tRNA halves. Averages of three experiments with SD values are shown. FIGS. 4C and 4D depict results from experiments showing total RNAs from HMDMs (A) or PHMDMs (B), treated with LPS or PGN, that were subjected to RT-qPCR for ANG and RPLP0 (control) mRNAs. HMDMs/PHMDMs without treatment served as a control. Averages of three experiments with SD values are shown. FIG. 4E depicts results showing alignment patterns of ChIP-seq reads (Zhao B, Barrera L A, Ersing I, Willox B, Schmidt S C, et al. Cell Rep 8: 1595-1606, 2014) around the ANG gene region (14q11.2: 21, 152-21,162 kb) for the indicated NF-fB family proteins. The Integrative Genomics Viewer was used for visualization. FIGS. 4F and 4G depict results from experiments showing total RNAs from HMDMs treated with LPS alone or LPS and JSH-23 (a NF-κB inhibitor) that were subjected to RT-qPCR for the indicated mRNAs.

FIG. 5, comprising FIGS. 5A-E, depicts exemplary results of experiments demonstrating abundant accumulation of tRNA halves in HMDM-secreted EVs. FIG. 5A depicts results of lysates from HMDMs and their secreted EVs that were subjected to western blots for the indicated EV- or non-EV-proteins. Cyto-c: cytochrome-c. FIG. 5B depicts results of isolated EVs (HMDM-EVs) that were analyzed by NTA. Particle images [left; Control (PBS): negative control] and size distribution profile (right) are shown. Representative raw video files from the NTA analyses are available in Supplementary Information. FIG. 5C depicts results of transmission electron microscopic evaluation for the isolated EVs, showing small vesicles with the expected size of EVs. Four representative EV images are shown. Scale bar, 100 nm. FIG. 5D depicts results of isolated EVs that were treated with RNase A and/or Triton X-100 and then subjected to stem-loop RT-qPCR and TaqMan RT-qPCR for quantification of each of the two indicated miRNAs and 5′-tRNA halves, respectively. Averages of three experiments with SD values are shown (***P<0.001; N. S.: Non-Significant, based on two-tailed t-Test). FIG. 5E depicts the expression of the miR-150 and 5′-tRNA^{HisGUG} half in HMDMs and their EVs, quantified by stem-loop/TaqMan RT-qPCRs, and their abundance was estimated based on the standard curves shown in FIG. 7. Averages of three experiments with SD values are shown.

FIG. 6 depicts a schematic of synthetic RNA sequences. Guanosine and uridine are shown in red circles. Modified nucleotides [dihydrouridine (D) and pseudouridine (Ψ)] are shown in green and blue circles, respectively.

FIG. 7 depicts exemplary results demonstrating standard curves for the quantification of miR-150 and 5′-tRNA^{HisGUG} half. Indicated amounts of synthetic RNAs were subjected to stem-loop/TaqMan RT-qPCRs. Proportional correlations of synthetic RNA input to the cycle threshold (Ct) were observed and used as standard curves for estimation of the expression levels of respective RNAs.

FIG. 8, comprising FIGS. 8A-G, depicts exemplary results of experiments demonstrating identification of 5′-tRNA halves expressed in HMDMs and their EVs by cP-RNA-seq. FIG. 8A depicts gel-purified 20-45-nt RNAs from LPS-treated HMDMs (untreated HMDMs: control) that were subjected to cP-RNA-seq, which amplified 140-160-bp cDNA products (5′-adapter, 55 bp; 3′-adapter, 63 bp; and thereby estimated inserted sequences, 22-42 bp). The cDNAs in the region highlighted by a line were purified and subjected to Illumina sequencing. FIG. 8B depicts HMDM EV-RNAs (#1 and #2: biological replicates) that were treated with wild-type T4 PNK (PNK WT) or its mutant (PNK M) lacking 3′-dephosphorylation activity and then subjected to Illumina cDNA amplification. Amplification of 140-160-bp cDNA products was dependent on PNK WT treatment. FIG. 8C depicts the ratio of HMDM library versus EV library for reads per million (RPM) of tRNA-derived RNA reads (tRNA), ribosomal RNA-derived RNA reads (rRNA), and mRNA-derived RNA reads (mRNA). FIG. 8D depicts the proportion of tRNA-derived cP-RNAs classified into the indicated subgroups of tRNA-derived ncRNAs. 5′- and 3′-tRFs are derived from 5′- and 3′-parts of tRNAs, respectively, while i-tRFs are derived from wholly internal parts of tRNAs (Shigematsu M, et al., 2015, Gene Regul SystBio, 9: 27-33). FIG. 8E depicts the proportion of the 5′-tRNA half-reads derived from respective cyto tRNA species. FIG. 8F depicts the ratio of HMDM library versus EV library for RPM of the indicated 5′-tRNA half species. FIG. 8G depicts the proportion of 5′-terminal (left) and 3′-terminal (right) nucleotides of the 5′-tRNA^{HisGUG} halves.

FIG. 9 depicts exemplary results demonstrating tRNA anticodon cleavage sites for generation of 5′-tRNA halves. Cleavage sites in the tRNA anticodon-loops were predicted based on the 3′-terminal positions of the 5′-tRNA halves. Anticodons are shown in green.

FIG. 10, comprising FIGS. 10A-B, depicts exemplary results of experiments demonstrating delivery of EV-5′-tRNA halves into endosomal TLR7.

FIGS. 10A and 10B depict fluorescent end-labeled, synthetic 5′-tRNA^{HisGUG} half (FIG. 10A) or 5′-tRNA^{GluCUC} half (FIG. 10B) that was transfected into HMDMs and observed in green. Scale bar, 20 μm.

FIG. 11, comprising FIGS. 11A-B, depicts exemplary results of experiments demonstrating delivery of EV-5′-tRNA halves into endosomes in recipient cells. EVs produced from host HMDMs containing the labeled 5′-tRNA^{HisGUG} half (FIG. 11A) or 5′-tRNA^{GluCUC} half (FIG. 11B) were isolated and applied to recipient HMDMs. Delivery of the labeled, EV-5′-tRNA half into endosomes is observed in green. Immunofluorescence staining of Rab7 is shown in red, and DNA was counterstained with DAPI in blue. Clear co-localization of the labeled 5′-tRNA halves and Rab7 is observed in merged panels. Scale bar, 100 μm.

FIG. 12, comprising FIGS. 12A-B, depicts exemplary results of experiments demonstrating delivery of EV-5′-tRNA halves into endosomal TLR7. FIGS. 12A and 12B depict EVs produced from host HMDMs containing the labeled 5′-tRNA^{HisGUG} half or 5′-tRNA^{GluCUC} half that were isolated and applied to recipient HMDMs. Delivery of the labeled, EV-5′-tRNA^{HisGUG} half (FIG. 12A) or EV-5′-tRNA^{GluCUC} half (FIG. 12B) into endosomes was observed in green. Immunofluorescence staining of TLR7 is shown in red, and DNA was counterstained with DAPI in blue. Scale bar, 100 μm. Clear co-localization of the labeled 5′-tRNA halves and TLR7 was observed.

FIG. 13, comprising FIGS. 13A-B, depicts exemplary results of experiments demonstrating activation of endosomal TLR by DOTAP-fused 5′-tRNA^{HisGUG} half. FIG. 13A depicts results of the synthetic 5′-tRNA halves, ssRNA40 (positive control), and its mutant (ssRNA40-M; negative control) that were transfected into HMDMs using DOTAP. Total RNAs from the cells were subjected to RT-qPCR for the indicated mRNAs. Averages of three experiments with SD values are shown (**P<0.01 and ***P<0.001; two-tailed t-Test). FIG. 13B depicts, after RNA transfection into HMDMs using DOTAP, culture medium that was subjected to ELISA for quantification of TNFα, and IL-1β.

FIG. 14 depicts exemplary results demonstrating that Lipofectamine-mediated transfection of 5′-tRNA^{HisGUG} half has no effect on immune response. Using RNAiMAX or Lipofectamine LTX (Thermo Fisher Scientific), the synthetic 5′-tRNA^{HisGUG} half and ssRNA40 were transfected into HMDMs. Total RNAs from the cells were subjected to RT-qPCR for the indicated mRNAs. Averages of three experiments with SD values are shown.

FIG. 15, comprising FIGS. 15A-B, depicts exemplary results of experiments demonstrating activation of endosomal TLR by DOTAP-fused 5′-tRNA^{HisGUG} half in PHMDMs. FIG. 15A depicts results of experiments performed as in FIG. 13A, but with PHMDMs. FIG. 15B depicts results of experiments performed as in FIG. 13B, but with PHMDMs.

FIG. 16, comprising FIGS. 16A-B, depicts exemplary results of experiments demonstrating activation of endosomal TLRs by various amounts of 5′-tRNA^{HisGUG} half. FIG. 16A depicts the indicated amounts of the synthetic 5′-tRNA^{HisGUG} half that were transfected into HMDMs using DOTAP. Total RNAs from the cells were subjected to RT-qPCR for the indicated mRNAs. Averages of three experiments with SD values are shown. FIG. 16B depicts, after the RNA transfection, culture medium that was subjected to ELISA for quantification of TNFα and IL-1β.

FIG. 17, comprising FIGS. 17A-B, depicts exemplary results of experiments demonstrating activation of endosomal TLR by DOTAP-fused 5′-tRNA^{HisGUG} half with modifications, but not full-length tRNA^{HisGUG}. FIG. 17A depicts results of experiments performed as in FIG. 13A, but using 5′-tRNA^{HisGUG} half with modifications (5′-HisGUG-Mod). FIG. 17B depicts results of experiments performed as in FIG. 13B, but using full-length tRNA^{HisGUG} (FL-HisGUG).

FIG. 18 depicts exemplary results demonstrating that 5′-tRNA^{HisGUG} half activates TLR7. In HMDMs, the expression of TLR7 or TLR8 was silenced by siRNAs and then the DOTAP-fused 5′-tRNA^{HisGUG} half or ssRNA40-M was transfected. Total RNAs from the cells were subjected to RT-qPCR for the indicated mRNAs. Averages of three experiments with SD values are shown (**P<0.01; two-tailed t-Test).

FIG. 19, comprising FIGS. 19A-C, depicts exemplary results of experiments demonstrating siRNA-mediated knockdown (KD) of TLR7 and TLR8 in HMDMs. FIG. 19A depicts results of HMDMs that were transfected with control siRNA (siControl) or siRNA targeting TLR7 (siTLR7) or TLR8 (siTLR8). To confirm the reduction of the targeted mRNA, total RNAs from the cells were subjected to RT-qPCR for TLR7 and TLR8 mRNAs (RPLP0: control). FIG. 19B depicts results of double KDs of TLR7 and TLR8 that were performed by simultaneously transfecting both siTLR7 and siTLR8, and reduction of the both mRNAs was confirmed by RT-qPCR. FIG. 19C depicts results of experiments where, in HMDMs, the expression of both TLR7 and TLR8 was silenced by siRNAs and then DOTAP-fused 5′-tRNA^{HisGUG} half or ssRNA40-M was transfected. Total RNAs from the cells was subjected to RT-qPCR for the indicated mRNAs.

FIG. 20, comprising FIGS. 20A-B, depicts exemplary results of experiments further demonstrating that 5′-tRNA^{HisGUG} half activates TLR7. FIG. 20A depicts results of lysates from two different TLR7KO THP-1 cell clones (#1 and #2), as well as from wild-type (WT) cells, that were subjected to western blots to confirm the depletion of TLR7 expression. FIG. 20B depicts results of the experiments as performed in FIG. 18, but using TLR7 KO cells (***P<0.001; two-tailed t-Test).

FIG. 21, comprising FIGS. 21A-C, depicts exemplary results of experiments demonstrating activation of TLR7 by endogenous EV-5′-tRNA^{HisGUG} half. FIG. 21A depicts results of EVs from HMDMs transfected with the indicated 5′-tRNA halves or ssRNA40-M that were isolated and applied to recipient HMDMs. Total RNAs from the cells were then subjected to RT-qPCR for the indicated mRNAs. Averages of three experiments with SD values are shown (*P<0.05, **P<0.01, and ***P<0.001; two-tailed t-Test). FIG. 21B depicts results showing the indicated synthetic RNAs, antisense oligonucleotides of the 5′-tRNA^{HisGUG} half (AS-oligo), the control oligonucleotides with scrambled sequences (Ctrl-oligo), or a mixture (the 5′-tRNA^{HisGUG} half was mixed with an equal amount of the oligonucleotides) that were subjected to DOTAP-mediated transfection into HMDMs, and indicated mRNAs were quantified. Averages of three experiments with SD values are shown. FIG. 21C depicts results of EVs from LPS-treated HMDMs that were mixed with DOTAP-fused AS- or Ctrl-oligo and applied to recipient HMDMs. Then, the indicated mRNA expression was quantified. Averages of three experiments with SD values are shown.

FIG. 22, comprising FIGS. 22A-B, depicts exemplary results of experiments demonstrating detection of tRNA halves in EVs isolated from human plasma. FIG. 22A depicts results of EVs that were isolated from human plasma and were analyzed by NTA. Representative size distribution profile is shown. FIG. 22B depicts results of isolated EVs that were treated with RNase A and/or Triton X-100 and then subjected to TaqMan RT-qPCR for quantification of 5′-tRNA halves. Averages of three experiments with SD values are shown (*P<0.05, **P<0.01, and ***P<0.001; two-tailed t-Test).

FIG. 23 depicts exemplary results demonstrating enhanced accumulation of tRNA halves in Mtb-infected patients. Human plasma sample (batch #1) was treated with RNase A and/or Triton X-100 and then subjected to TaqMan RT-qPCR for quantification of 5′-tRNA halves. Averages of three experiments with SD values are shown (***P<0.001; two-tailed t-Test).

FIG. 24 depicts exemplary results further demonstrating detection of tRNA halves in EVs isolated from human plasma. Human plasma samples (batches #2-4) were treated with RNase A and/or Triton X-100 and then subjected to TaqMan RT-qPCR for quantification of 5′-tRNA halves. Averages of three experiments with SD values are shown.

FIG. 25 depicts exemplary results further demonstrating enhanced accumulation of tRNA halves in Mtb-infected patients. RNAs isolated from plasma samples of healthy subjects (n=8) or Mtb-infected patients (n=6) were subjected to TaqMan RT-qPCR for the indicated 5′-tRNA halves. The quantified 5′-tRNA half levels were normalized to spike-in synthetic mouse piR-3 levels.

FIG. 26, comprising FIGS. 26A-B, depicts a proposed model for 5′-tRNA half-mediated immune response. FIG. 26A depicts a model in which surface TLR stimulation culminates in activation of NF-fB, leading to upregulation of ANG, which cleaves the anticodon-loops of tRNAs. The resultant 5′-tRNA halves are secreted by being packaged into EVs and function as signaling molecules. FIG. 26B depicts an extension of this model in which EV-5′-tRNA halves are delivered into endosomes in recipient cells and activate TLR7, which promotes the immune response.

FIG. 27, comprising FIGS. 27A-B, depicts exemplary results demonstrating that 5′-tRNA^{ValCAC/ValAAC} halves functionally activate endosomal TLR7 for cytokine secretion. FIG. 27A depicts a schematic representation of experimental procedures. HMDMs were primed with interferon γ and then transfected with the selected 5′-tRNA half using the cationic liposome 1,2-dioleoyloxy-3-trimethylammonium-propane (DOTAP) which mimics EVs. As a negative control, ssRNA40-M, (in which U is replaced with A of 20-nt HIV-1-derived ssRNA termed ssRNA40)3 was also transfected. FIG. 27B depicts cytokine profiling upon transfection of the indicated RNAs.

FIG. 28, comprising FIGS. 28A-B, depicts exemplary results demonstrating that activation of endosomal TLR7 by 5′-tRNA halves prior to the infection enhances bacterial elimination. After 5′-tRNA^{HisGUG} half- or 5′-tRNA^{ValCAC} half-mediated TLR activation, HMDMs were infected with E-coli with different multiplicities of infection (MOI) and incubated. FIG. 28A depicts representative plate images after HMDMs were lysed, plated on LB agar plates, and incubated once more for colony forming assays. FIG. 28B depicts the quantification of colony forming units (CFU) per plate obtained from three experiments.

### DETAILED DESCRIPTION

The present invention relates to variant tRNA molecules, fragments of tRNA molecules and methods of use thereof to modulate toll like receptor (TLR) signaling, for immunotherapy and for other therapeutic applications.

In various embodiments, the variant tRNA molecule comprises a mutation relative to a wild-type (WT) tRNA molecule. In some embodiments, the WT tRNA molecule is tRNA^{HisGUG}, tRNA^{GluCUC}, tRNA^{ValCAC}, tRNA^{GlyYGCC}, tRNA^{ValAAC}, tRNA^{GluUUC}, tRNA^{LysCUU}, tRNA^{AspGUC}, tRNA^{MetCAU}, tRNA^{ProAGG}, tRNA^{LeuCAG}, tRNA^{ArgUCU}, tRNA^{LysUUU}, tRNA^{ValUAC}, tRNA^{GlnCUG}, tRNA^{ArgCCG}, tRNA^{ArgACG}, tRNA^{LeuUAA}, tRNA^{ArgUCG}, tRNA^{AsnGUU}, tRNA^{AlaCGC}, tRNA^{LeuAAG}, tRNA^{ThrUGU}, tRNA^{AlaAGC}, tRNA^{LeuCAA}, tRNA^{PheGAA}, tRNA^{GlnUUG}, tRNA^{TrpCCA}, tRNA^{SerGCU}, tRNA^{CysGCA}, tRNA^{IleAAU}, tRNA^{ArgCCU}, tRNA^{SerAGA}, tRNA^{GlyUCC}, tRNA^{LeuUAG}, tRNA^{AlaUGC}, or tRNA^{ThrCGU}. In some embodiments, the WT tRNA molecule is tRNA^{HisGUG}, tRNA^{ValCAC}, or tRNA^{ValAAC}. In some embodiments, fragment of the tRNA molecule comprises the 5′portion of tRNA^{HisGUG}, tRNA^{ValCAC} or tRNA^{ValAAC}.

In various embodiments, the fragment of the tRNA molecule comprises the 5′portion of the tRNA molecule. In various embodiments, the fragment of the tRNA molecule comprises SEQ ID NO:1, SEQ ID NO:3, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO: 69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO: 79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91 or SEQ ID NO:92, or a fragment or variant thereof.

In various embodiments, the variant tRNA molecule, or tRNA molecule fragment specifically binds to and activates at least one TLR. In some embodiments, a variant tRNA molecule, or a fragment thereof, that specifically binds to and activates a TLR, is useful for inducing, enhancing, or promoting TLR signaling activity. In various embodiments, the TLR is TLR7 or TLR8.

In some embodiments, the variant tRNA molecule or tRNA molecule fragment, is useful for the treatment and prevention of a disease or disorder. In various embodiments, the disease or disorder is cancer or an infectious disease. Thus, in some embodiments, the invention is a composition comprising at least one variant tRNA molecule or tRNA molecule fragment. In other embodiments, the invention is a method of administering at least one variant tRNA molecule or tRNA molecule fragment, to treat or prevent a disease or disorder, such as, but not limited to, cancer, or an infectious disease.

In some embodiments, the variant tRNA molecule or tRNA molecule fragment is a mammalian variant tRNA molecule or tRNA molecule fragment. In some embodiments, the variant tRNA molecule or tRNA molecule fragment is a human variant tRNA molecule or tRNA molecule fragment.

In various embodiments, the compositions and methods of the invention include compositions and methods for treating and preventing disease and disorders, such as cancer or infectious disease. In some embodiments, a method comprises administering to a subject in need thereof a composition comprising at least one variant tRNA molecule or tRNA molecule fragment.

In some embodiments, a method comprises administering to a subject in need thereof a composition comprising at least one variant tRNA molecule or tRNA molecule fragment, and administering to the subject a composition comprising an additional agent. Additional agents that can be administered include, but are not limited to, an altered T-cell, a chimeric antigen receptor T-cell (CAR-T), an antigen, a vaccine, an antibody, an immune checkpoint inhibitor, a small molecule, a chemotherapeutic agent, or a stem cell. In some embodiments, a composition comprising at least one variant tRNA molecule or tRNA molecule fragment is used in a method to increase immune system activity before, during, or after infection by a bacterium, virus, or other pathogen. In some embodiments, a composition comprising at least one variant tRNA molecule or tRNA molecule fragment is used in a method to increase the number and/or activity of immune cells in vitro, in vivo or ex vivo, such as the number and/or activity of T cells, NK cells, and/or myeloid cells.

### Definitions

Unless defined otherwise, all technical and scientific terms used herein have the same meaning as commonly understood by one of ordinary skill in the art to which this invention belongs.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of 20%, ±10%, ±5%, ±1%, or ±0.1% from the specified value, as such variations are appropriate to perform the disclosed methods.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. The antibodies in the present invention may exist in a variety of forms including, for example, polyclonal antibodies, monoclonal antibodies, intracellular antibodies (“intrabodies”), Fv, Fab and F(ab)2, as well as single chain antibodies (scFv), heavy chain antibodies, such as camelid antibodies, synthetic antibodies, chimeric antibodies, and a humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, New York; Houston et al., 1988, Proc. Natl. Acad. Sci. USA 85:5879-5883; Bird et al., 1988, Science 242:423-426).

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations. κ and λ light chains refer to the two major antibody light chain isotypes.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

As used herein, an “immunoassay” refers to any binding assay that uses an antibody capable of binding specifically to a target molecule to detect and quantify the target molecule.

By the term “specifically binds,” as used herein, is meant a molecule that recognizes and binds to a specific receptor, such as a TLR. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species.

By the term “applicator,” as the term is used herein, is meant any device including, but not limited to, a hypodermic syringe, a pipette, an iontophoresis device, a patch, and the like, for administering the compositions of the invention to a subject.

“Cancer,” as used herein, refers to the abnormal growth or division of cells. Generally, the growth and/or life span of a cancer cell exceeds, and is not coordinated with, that of the normal cells and tissues around it. Cancers may be benign, pre-malignant or malignant. Cancer occurs in a variety of cells and tissues, including the oral cavity (e.g., mouth, tongue, pharynx, etc.), digestive system (e.g., esophagus, stomach, small intestine, colon, rectum, liver, bile duct, gall bladder, pancreas, etc.), respiratory system (e.g., larynx, lung, bronchus, etc.), bones, joints, skin (e.g., basal cell, squamous cell, meningioma, etc.), breast, genital system, (e.g., uterus, ovary, prostate, testis, etc.), urinary system (e.g., bladder, kidney, ureter, etc.), eye, nervous system (e.g., brain, etc.), endocrine system (e.g., thyroid, etc.), and hematopoietic system (e.g., lymphoma, myeloma, leukemia, acute lymphocytic leukemia, chronic lymphocytic leukemia, acute myeloid leukemia, chronic myeloid leukemia, etc.).

The term “coding sequence,” as used herein, means a sequence of a nucleic acid or its complement, or a part thereof, that can be transcribed and/or translated to produce the mRNA and/or the polypeptide or a fragment thereof. Coding sequences include exons in a genomic DNA or immature primary RNA transcripts, which are joined together by the cell's biochemical machinery to provide a mature mRNA. The anti-sense strand is the complement of such a nucleic acid, and the coding sequence can be deduced therefrom. In contrast, the term “non-coding sequence,” as used herein, means a sequence of a nucleic acid or its complement, or a part thereof, that is not translated into amino acid in vivo, or where tRNA does not interact to place or attempt to place an amino acid. Non-coding sequences include both intron sequences in genomic DNA or immature primary RNA transcripts, and gene-associated sequences such as promoters, enhancers, silencers, and the like.

As used herein, the terms “complementary” or “complementarity” are used in reference to polynucleotides (i.e., a sequence of nucleotides) related by the base-pairing rules. For example, the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

An “effective amount” as used herein, means an amount which provides a therapeutic, prophylactic, or other desired benefit.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein, the term “fragment,” as applied to a nucleic acid, refers to a subsequence of a larger nucleic acid. A “fragment” of a nucleic acid can be at least about 15 nucleotides in length; for example, at least about 50 nucleotides to about 100 nucleotides; at least about 100 to about 500 nucleotides, at least about 500 to about 1000 nucleotides; at least about 1000 nucleotides to about 1500 nucleotides; about 1500 nucleotides to about 2500 nucleotides; or about 2500 nucleotides (and any integer value in between). As used herein, the term “fragment,” as applied to a protein, polypeptide or peptide, refers to a subsequence of a larger protein, polypeptide or peptide. A “fragment” of a protein, polypeptide, or peptide can be at least about 5 amino acids in length; for example, at least about 10 amino acids in length; at least about 20 amino acids in length; at least about 50 amino acids in length; at least about 100 amino acids in length; at least about 200 amino acids in length; or at least about 300 amino acids in length (and any integer value in between).

The term “gene” refers to a nucleic acid (e.g., DNA) sequence that includes coding sequences necessary for the production of a polypeptide, precursor, or RNA (e.g., mRNA). The polypeptide may be encoded by a full-length coding sequence or by any portion of the coding sequence so long as the desired activity or functional property (e.g., enzymatic activity, receptor binding, signal transduction, immunogenicity, etc.) of the full-length or fragment is retained. The term also encompasses the coding region of a structural gene and the sequences located adjacent to the coding region on both the 5′ and 3′ ends for a distance of about 2 kb or more on either end such that the gene corresponds to the length of the full-length mRNA and 5′ regulatory sequences which influence the transcriptional properties of the gene. Sequences located 5′ of the coding region and present on the mRNA are referred to as 5′-untranslated sequences. The 5′-untranslated sequences usually contain the regulatory sequences. Sequences located 3′ or downstream of the coding region and present on the mRNA are referred to as 3′-untranslated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

“Homologous”, “identical,” or “identity” as used herein in the context of two or more nucleic acids or polypeptide sequences means that the sequences have a specified percentage of residues that are the same over a specified region. The percentage can be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of the single sequence are included in the denominator but not the numerator of the calculation. When comparing DNA and RNA, thymine (T) and uracil (U) can be considered equivalent. Identity can be performed manually or by using a computer sequence algorithm such as BLAST or BLAST 2.0.

“Immune response,” as the term is used herein, means a process that results in the activation and/or invocation of an effector function in either the T cells, B cells, natural killer (NK) cells, and/or antigen-presenting cells (APCs). Thus, an immune response, as would be understood by the skilled artisan, includes, but is not limited to, any detectable antigen-specific or allogeneic activation of a helper T cell or cytotoxic T cell response, production of antibodies, T cell-mediated activation of allergic reactions, macrophage infiltration, and the like.

“Instructional material,” as that term is used herein, includes a publication, a recording, a diagram, or any other medium of expression which can be used to communicate the usefulness of the nucleic acid, peptide, polypeptide, and/or compound of the invention in the kit for identifying or alleviating or treating the various diseases or disorders recited herein. Optionally, or alternately, the instructional material may describe one or more methods of identifying or alleviating the diseases or disorders in a cell or a tissue of a subject. The instructional material of the kit may, for example, be affixed to a container that contains the nucleic acid, polypeptide, and/or compound of the invention or be shipped together with a container that contains the nucleic acid, polypeptide, and/or compound. Alternatively, the instructional material may be shipped separately from the container with the intention that the recipient uses the instructional material and the compound cooperatively.

“Isolated” means altered or removed from the natural state. For example, a nucleic acid or a polypeptide naturally present in a living animal is not “isolated,” but the same nucleic acid or polypeptide partially or completely separated from the coexisting materials of its natural state is “isolated.” An isolated nucleic acid or protein can exist in substantially purified form, or can exist in a non-native environment such as, for example, a host cell.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

The term “label” when used herein refers to a detectable compound or composition that is conjugated directly or indirectly to a probe to generate a “labeled” probe. The label may be detectable by itself (e.g., radioisotope labels or fluorescent labels) or, in the case of an enzymatic label, may catalyze chemical alteration of a substrate compound or composition that is detectable (e.g., avidin-biotin). In some instances, primers can be labeled to detect a PCR product.

By the term “modulating,” as used herein, is meant mediating a detectable increase or decrease in the activity and/or level of a mRNA, polypeptide, or a response in a subject compared with the activity and/or level of a mRNA, polypeptide or a response in the subject in the absence of a treatment or compound, and/or compared with the activity and/or level of a mRNA, polypeptide, or a response in an otherwise identical but untreated subject. The term encompasses activating, inhibiting and/or otherwise affecting a native signal or response thereby mediating a beneficial therapeutic, prophylactic, or other desired response in a subject, for example, a human.

A “mutation,” “mutant,” or “variant,” as used herein, refers to a change in nucleic acid or polypeptide sequence relative to a reference sequence (which may be a naturally-occurring normal or the “wild-type” sequence), and includes translocations, deletions, insertions, and substitutions/point mutations. A “mutant” or “variant” as used herein, refers to either a nucleic acid or protein comprising a mutation.

A “nucleic acid” refers to a polynucleotide and includes poly-ribonucleotides and poly-deoxyribonucleotides. Nucleic acids according to the present invention may include any polymer or oligomer of pyrimidine and purine bases, preferably cytosine, thymine, and uracil, and adenine and guanine, respectively. (See Albert L. Lehninger, Principles of Biochemistry, at 793-800 (Worth Pub. 1982) which is herein incorporated in its entirety for all purposes). Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

An “oligonucleotide” or “polynucleotide” is a nucleic acid ranging from at least 2, preferably at least 8, 15 or 25 nucleotides in length, but may be up to 50, 100, 1000, or 5000 nucleotides long or a compound that specifically hybridizes to a polynucleotide. Polynucleotides include sequences of deoxyribonucleic acid (DNA) or ribonucleic acid (RNA) or mimetics thereof which may be isolated from natural sources, recombinantly produced or artificially synthesized. A further example of a polynucleotide of the present invention may be a peptide nucleic acid (PNA). (See U.S. Pat. No. 6,156,501 which is hereby incorporated by reference in its entirety.) The invention also encompasses situations in which there is a nontraditional base pairing such as Hoogsteen base pairing which has been identified in certain tRNA molecules and postulated to exist in a triple helix. “Polynucleotide” and “oligonucleotide” are used interchangeably in this disclosure. It will be understood that when a nucleotide sequence is represented herein by a DNA sequence (e.g., A, T, G, and C), this also includes the corresponding RNA sequence (e.g., A, U, G, C) in which “U” replaces “T”.

The terms “patient,” “subject,” “individual,” and the like are used interchangeably herein, and refer to any animal, or cells thereof whether in vivo, in vitro or in situ, amenable to the methods described herein. In certain non-limiting embodiments, the patient, subject or individual is a human.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, mutant polypeptides, variant polypeptides, or a combination thereof.

As used herein, “polynucleotide” includes cDNA, RNA, DNA/RNA hybrid, antisense RNA, ribozyme, genomic DNA, synthetic forms, and mixed polymers, both sense and antisense strands, and may be chemically or biochemically modified to exhibit non-natural or derivatized, synthetic, or semi-synthetic nucleotide bases. Also, contemplated are alterations of a wild type or synthetic gene, including but not limited to deletion, insertion, substitution of one or more nucleotides, or fusion to other polynucleotide sequences.

To “prevent” a disease or disorder as the term is used herein, means to reduce the severity or frequency of at least one sign or symptom of a disease or disorder that is to be experienced by a subject.

“Sample” or “biological sample” as used herein means a biological material isolated from a subject. The biological sample may contain any biological material suitable for detecting a mRNA, polypeptide or other marker of a physiologic or pathologic process in a subject, and may comprise fluid, tissue, cellular and/or non-cellular material obtained from the individual.

As used herein, “substantially purified” refers to being essentially free of other components. For example, a substantially purified polypeptide is a polypeptide which has been separated from other components with which it is normally associated in its naturally occurring state.

As used herein, the terms “therapy” or “therapeutic regimen” refer to those activities taken to prevent, treat or alter a disease or disorder, e.g., a course of treatment intended to reduce or eliminate at least one sign or symptom of a disease or disorder using pharmacological, surgical, dietary and/or other techniques. A therapeutic regimen may include a prescribed dosage of one or more compounds or surgery. Therapies will most often be beneficial and reduce or eliminate at least one sign or symptom of the disorder or disease state, but in some instances the effect of a therapy will have non-desirable or side-effects. The effect of therapy will also be impacted by the physiological state of the subject, e.g., age, gender, genetics, weight, other disease conditions, etc.

The term “therapeutically effective amount” refers to the amount of the subject compound or composition that will elicit the biological, physiologic, clinical or medical response of a cell, tissue, organ, system, or subject that is being sought by the researcher, veterinarian, medical doctor or other clinician. The term “therapeutically effective amount” includes that amount of a compound or composition that, when administered, is sufficient to prevent development of, or treat to some extent, one or more of the signs or symptoms of the disorder or disease being treated. The therapeutically effective amount will vary depending on the compound or composition, the disease and its severity and the age, weight, etc., of the subject to be treated.

To “treat” a disease or disorder as the term is used herein, means to reduce the frequency or severity of at least one sign or symptom of a disease or disorder experienced by a subject. The terms “treatment”, “treating”, “treat” and the like are used herein to generally refer to obtaining a desired pharmacologic and/or physiologic effect. The effect can be prophylactic in terms of completely or partially preventing a disease or symptom(s) thereof and/or may be therapeutic in terms of a partial or complete stabilization or cure for a disease and/or adverse effect attributable to the disease. The term “treatment” encompasses any treatment of a disease in a mammal, particularly a human, and includes: (a) preventing the disease and/or symptom(s) from occurring in a subject who may be predisposed to the disease or symptom but has not yet been diagnosed as having it; (b) inhibiting the disease and/or symptom(s), e.g., slowing or arresting their development (e.g., halting the growth of tumors, slowing the rate of tumor growth, halting the rate of cancer cell proliferation, and the like); or (c) relieving the disease symptom(s), i.e., causing regression of the disease and/or symptom(s) (e.g., causing decrease in tumor size, reducing the number of cancer cells present, and the like). Those in need of treatment include those already inflicted (e.g., those with cancer, those with an infection, those with a metabolic disorder, those with macular degeneration, etc.) as well as those in which prevention is desired (e.g., those with increased susceptibility to cancer, those with an increased likelihood of infection, those suspected of having cancer, those suspected of harboring an infection, those with increased susceptibility for metabolic disease, those with increased susceptibility for macular degeneration, etc.).

As used herein, the term “wild-type” refers to a gene or gene product isolated from a naturally occurring source. A wild-type gene is that which is most frequently observed in a population and is thus arbitrarily designated the “normal” or “wild-type” form of the gene. In contrast, the term “modified,” “variant,” or “mutant” refers to a gene or gene product that possesses modifications in sequence and/or functional properties (i.e., altered characteristics) when compared to the wild-type gene or gene product.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

In some embodiments, the compositions and methods of the invention comprise a variant or fragment of a tRNA molecule. In some embodiments, the variant or fragment of a tRNA molecule functions as an agonist of at least one toll-like receptor (TLR), such as TLR7 or TLR8. In some embodiments, the variant or fragment of a tRNA molecule is a molecule that promotes TLR signaling. In some embodiments, the variant or fragment of a tRNA molecule is a molecule that is able to bind and to signal through the TLR.

In some embodiments, the variant or fragment of a tRNA molecule is a variant or fragment of at least one of tRNA^{HisGUG}, tRNA^{GluCUC}, tRNA^{ValCAC}, tRNA^{GlyGCC}, tRNA^{ValAAC}, tRNA^{GluUUC}, tRNA^{LysCUU}, tRNA^{AspGUC}, tRNA^{MetCAU}, tRNA^{ProAGG}, tRNA^{LeuCAG}, tRNA^{ArgUCU}, tRNA^{LysUUU}, tRNA^{ValUAC}, tRNA^{GlnCUG}, tRNA^{ArgCCG}, tRNA^{ArgACG}, tRNA^{LeuUAA}, tRNA^{ArgUCG}, tRNA^{AsnGUU}, tRNA^{AlaCGC}, tRNA^{LeuAAG}, tRNA^{ThrUGU}, tRNA^{AlaAGC}, tRNA^{LeuCAA}, tRNA^{PheGAA}, tRNA^{GlnUUG}, tRNA^{TrpCCA}, tRNA^{SerGCU}, tRNA^{CysGCA}, tRNA^{IleAAU}, tRNA^{ArgCCU}, tRNA^{SerAGA}, tRNA^{GlyUCC}, tRNA^{LeuUAG}, tRNA^{AlaUGC}, or tRNA^{ThrCGU}. In some embodiments, the a variant or fragment of a tRNA molecule comprises a 5′ fragment of a tRNA molecule. In some embodiments, the a variant or fragment of a tRNA molecule comprises a 5′ fragment of tRNA^{HisGUG}, tRNA^{ValCAC}, or tRNA^{ValAAC}

### Compositions

In some embodiments, the invention is a variant or fragment of a tRNA molecule. In one embodiment, the variant or fragment tRNA molecule specifically binds to at least one TLR, and promotes TLR signaling.

In some embodiments the variant tRNA molecule comprises at least one mutation relative to a native or “wild type” tRNA molecule. In various embodiments, the variant tRNA molecule comprises at least 50%, 60%, 70%, 80%, 85%, 90%, 95%, 96%, 97%, 98%, 99% or more than 99% sequence identity to a native or “wild type” tRNA molecule. In some embodiments the variant tRNA molecule comprises at least one modified nucleotide relative to a naturally occurring or “wild type” tRNA molecule. In various embodiments, the variant tRNA molecule comprises at least 2, 3, 4, 5, 6, 7, 8, 9, or more than 10 modified nucleotides relative to a naturally occurring or “wild type” tRNA molecule.

The variant tRNA molecule of the invention may include phosphodiester linkages or modified linkages such as phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate or sulfone linkages, and combinations of such linkages. The variant tRNA molecule of the invention also specifically includes nucleic acids composed of bases other than adenine, guanine, cytosine and uracil. For example, in some embodiments, the variant tRNA molecule comprises a dihydrouridine, pseudouridine, or a 2′-O-methylated nucleotide.

In some embodiments the fragment of a tRNA molecule comprises at least 30% of the full length sequence of a native or “wild type” tRNA molecule. In various embodiments, the fragment of a tRNA molecule comprises at least 35%, 40%, 45%, 50%, 55%, 60%, 65%, 70%, 75%, 80%, 85%, 90%, 95% 96%, 97%, 98%, or at least 99% the full length sequence of a native or “wild type” tRNA molecule. In various embodiments, the fragment of a tRNA molecule comprises less than 35%, 40%, 45%, 50%, 55%, 60%, 65%, 70%, 75%, 80%, 85%, 90%, 95% 96%, 97%, 98%, or less than 99% the full length sequence of a native or “wild type” tRNA molecule. In one embodiment, the fragment of a tRNA molecule comprises about 50% of the full length sequence of a native or “wild type” tRNA molecule.

In some embodiments the fragment of a tRNA molecule comprises at least the 4 nucleotides beginning at the 5′end of the full length sequence of a native or “wild type” tRNA molecule. In various embodiments, the fragment of a tRNA molecule comprises at least 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 50 or more than 50 nucleotides beginning at the 5′end of the full length sequence of a native or “wild type” tRNA molecule. In one embodiment, the fragment of a tRNA molecule comprises the 5′ half of the full length sequence of a native or “wild type” tRNA molecule.

In some embodiments the invention provides a variant of a fragment of a tRNA molecule. In one embodiment, the variant of the fragment comprises at least 50%, 60%, 70%, 80%, 85%, 90%, 95%, 96%, 97%, 98%, 99% or more than 99% sequence identity over at least 30% of the full length sequence of a native or “wild type” tRNA molecule. In one embodiment, the variant of the fragment comprises at least 50%, 60%, 70%, 80%, 85%, 90%, 95%, 96%, 97%, 98%, 99% or more than 99% sequence identity over at least 35%, 40%, 45%, 50%, 55%, 60%, 65%, 70%, 75%, 80%, 85%, 90%, 95% 96%, 97%, 98%, or at least 99% the full length sequence of a native or “wild type” tRNA molecule. In one embodiment, the variant of the fragment comprises at least 50%, 60%, 70%, 80%, 85%, 90%, 95%, 96%, 97%, 98%, 99% or more than 99% sequence identity over less than 35%, 40%, 45%, 50%, 55%, 60%, 65%, 70%, 75%, 80%, 85%, 90%, 95% 96%, 97%, 98%, or less than 99% the full length sequence of a native or “wild type” tRNA molecule. In one embodiment, the variant of the fragment comprises at least 50%, 60%, 70%, 80%, 85%, 90%, 95%, 96%, 97%, 98%, 99% or more than 99% sequence identity over about 50% of the full length sequence of a native or “wild type” tRNA molecule.

In one embodiment, the wildtype tRNA molecule of the invention comprises tRNA^{HisGUG}, tRNA^{GluCUC}, tRNA^{ValCAC}, tRNA^{GlyGCC}, tRNA^{ValAAC}, tRNA^{GluUUC}, tRNA^{LysCUU}, tRNA^{AspGUC}, tRNA^{MetCAU}, tRNA^{ProAGG}, tRNA^{LeuCAG}, tRNA^{ArgUCU}, tRNA^{LysUUU}, tRNA^{ValUAC}, tRNA^{GlnCUG}, tRNA^{ArgCCG}, tRNA^{ArgACG}, tRNA^{LeuUAA}, tRNA^{ArgUCG}, tRNA^{AsnGUU}, tRNA^{AlaCGC}, tRNA^{LeuAAG}, tRNA^{ThrUGU}, tRNA^{AlaAGC}, tRNA^{LeuCAA}, tRNA^{PheGAA}, tRNA^{GlnUUG}, tRNA^{TrpCCA}, tRNA^{SerGCU}, tRNA^{CysGCA}, tRNA^{IleAAU}, tRNA^{ArgCCU}, tRNA^{SerAGA}, tRNA^{GlyUCC}, tRNA^{LeuUAG}, tRNA^{AlaUGC}, or tRNA^{ThrCGU}. In one embodiment, the wildtype tRNA molecule of the invention comprises tRNA^{HisGUG}, tRNA^{GluCUC}, tRNA^{ValCAC}, tRNA^{GlyGCC}, or tRNA^{ValAAC}. Therefore, in some embodiments, the invention provides a fragment of tRNA^{HisGUG}, tRNA^{GluCUC} tRNA^{ValCAC}, tRNA^{GlyGCC}, tRNA^{ValAAC}, tRNA^{GluUUC}, tRNA^{LysCUU}, tRNA^{AspGUC}, tRNA^{MetCAU}, tRNA^{ProAGG}, tRNA^{LeuCAG}, tRNA^{ArgUCU}, tRNA^{LysUUU}, tRNA^{ValUAC}, tRNA^{GlnCUG}, tRNA^{ArgCCG}, tRNA^{ArgACG}, tRNA^{LeuUAA}, tRNA^{ArgUCG}, tRNA^{AsnGUU}, tRNA^{AlaCGC}, tRNA^{LeuAAG}, tRNA^{ThrUGU}, tRNA^{AlaAGC}, tRNA^{LeuCAA}, tRNA^{PheGAA}, tRNA^{GlnUUG}, tRNA^{TrpCCA}, tRNA^{SerGCU}, tRNA^{CysGCA}, tRNA^{IleAAU}, tRNA^{ArgCCU}, tRNA^{SerAGA}, tRNA^{GlyUCC}, tRNA^{LeuUAG}, tRNA^{AlaUGC}, or tRNA^{ThrCGU}. In some embodiments, the invention provides a variant of tRNA^{HisGUG}, tRNA^{GluCUC}, tRNA^{ValCAC}, tRNA^{GlyGCC}, tRNA^{ValAAC}, tRNA^{GluUUC}, tRNA^{LysCUU}, tRNA^{AspGUC}, tRNA^{MetCAU}, tRNA^{ProAGG}, tRNA^{LeuCAG}, tRNA^{ArgUCU}, tRNA^{LysUUU}, tRNA^{ValUAC}, tRNA^{GlnCUG}, tRNA^{ArgCCG}, tRNA^{ArgACG}, tRNA^{LeuUAA}, tRNA^{ArgUCG}, tRNA^{AsnGUU}, tRNA^{AlaCGC}, tRNA^{LeuAAG}, tRNA^{ThrUGU}, tRNA^{AlaAGC}, tRNA^{LeuCAA}, tRNA^{PheGAA}, tRNA^{GlnUUG}, tRNA^{TrpCCA}, tRNA^{SerGCU}, tRNA^{CysGCA}, tRNA^{IleAAU}, tRNA^{ArgCCU}, tRNA^{SerAGA}, tRNA^{GlyUCC}, tRNA^{LeuUAG}, tRNA^{AlaUGC}, or tRNA^{ThrCGU}. In some embodiments, the invention provides a variant of a fragment of tRNA^{HisGUG}, tRNA^{GluCUC}, tRNA^{ValCAC}, tRNA^{GlyGCC}, tRNA^{ValAAC}, tRNA^{GluUUC}, tRNA^{LysCUU}, tRNA^{AspGUC}, tRNA^{MetCAU}, tRNA^{ProAGG}, tRNA^{LeuCAG}, tRNA^{ArgUCU}, tRNA^{LysUUU}, tRNA^{ValUAC}, tRNA^{GlnCUG}, tRNA^{ArgCCG}, tRNA^{ArgACG}, tRNA^{LeuUAA}, tRNA^{ArgUCG}, tRNA^{AsnGUU}, tRNA^{AlaCGC}, tRNA^{LeuAAG}, tRNA^{ThrUGU}, tRNA^{AlaAGC}, tRNA^{LeuCAA}, tRNA^{PheGAA}, tRNA^{GlnUUG}, tRNA^{TrpCCA}, tRNA^{SerGCU}, tRNA^{CysGCA}, tRNA^{IleAAU}, tRNA^{ArgCCU}, tRNA^{SerAGA}, tRNA^{GlyUCC}, tRNA^{LeuUAG}, tRNA^{AlaUGC}, or tRNA^{ThrCGU}.

In one embodiment, the fragment of tRNA comprises SEQ ID NO:1, SEQ ID NO:3, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO: 76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91 or SEQ ID NO:92 or a variant thereof. In one embodiment, the variant of the fragment of tRNA comprises a sequence having at least 1, 2, 3, 4, 5, 6, 7, 8, 9, 10 or more than 10 modified nucleotides. In one embodiment, the variant of the fragment of tRNA comprises a sequence having at least 80%, 85%, 90%, 91%, 92%, 93%, 94%, 95%, 96%, 97%, 98%, 99% or more than 99% identity to SEQ ID NO:1, SEQ ID NO:3, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO: 67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO: 77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91 or SEQ ID NO:92.

In one exemplary embodiment, the fragment of tRNA^{HisGUG} comprises SEQ ID NO:1 or a fragment or variant thereof. In one embodiment, the fragment of tRNA^{HisGUG} comprises a sequence having at least 4, 5, 6, 7, 8, 9, 10, 11, 12, 13, 14, 15, 16, 17, 18, 19, 20, 21, 22, 23, 24, 25, 26, 27, 28, 29, 30 or more than 30 nucleotides of SEQ ID NO:1. In one embodiment, the variant of the fragment of tRNA^{HisGUG} comprises a sequence having at least 1, 2, 3, 4, 5, 6, 7, 8, 9, 10 or more than 10 modified nucleotides. In one embodiment, the variant of the fragment of tRNA^{HisGUG} comprises a sequence having at least 80%, 85%, 90%, 91%, 92%, 93%, 94%, 95%, 96%, 97%, 98%, 99% or more than 99% identity to SEQ ID NO:1. In one embodiment, the variant of the fragment of tRNA^{HisGUG} comprises SEQ ID NO:2.

In one exemplary embodiment, the fragment of tRNA^{ValCAC} comprises SEQ ID NO:57 or a variant thereof. In one embodiment, the fragment of tRNA^{ValCAC} comprises a sequence having at least 4, 5, 6, 7, 8, 9, 10, 11, 12, 13, 14, 15, 16, 17, 18, 19, 20, 21, 22, 23, 24, 25, 26, 27, 28, 29, 30 or more than 30 nucleotides of SEQ ID NO:57. In one embodiment, the variant of the fragment of tRNA^{ValCAC} comprises a sequence having at least 1, 2, 3, 4, 5, 6, 7, 8, 9, 10 or more than 10 modified nucleotides. In one embodiment, the variant of the fragment of tRNA^{ValCAC} comprises a sequence having at least 80%, 85%, 90%, 91%, 92%, 93%, 94%, 95%, 96%, 97%, 98%, 99% or more than 99% identity to SEQ ID NO:57.

In one exemplary embodiment, the fragment of tRNA^{ValAAC} comprises SEQ ID NO:60 or a variant thereof. In one embodiment, the fragment of tRNA^{ValAAC} comprises a sequence having at least 4, 5, 6, 7, 8, 9, 10, 11, 12, 13, 14, 15, 16, 17, 18, 19, 20, 21, 22, 23, 24, 25, 26, 27, 28, 29, 30 or more than 30 nucleotides of SEQ ID NO:60.

In one embodiment, the variant of the fragment of tRNA^{ValAAC} comprises a sequence having at least 1, 2, 3, 4, 5, 6, 7, 8, 9, 10 or more than 10 modified nucleotides. In one embodiment, the variant of the fragment of tRNA^{ValAAC} comprises a sequence having at least 80%, 85%, 90%, 91%, 92%, 93%, 94%, 95%, 96%, 97%, 98%, 99% or more than 99% identity to SEQ ID NO:60.

In some embodiments, the invention provides an isolated nucleic acid molecule encoding the variant or fragment of a tRNA molecule of the invention. In some embodiments the isolated nucleic acid molecule encoding the variant or fragment of a tRNA molecule of the invention can be obtained using any of the many recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

The isolated nucleic acid may comprise any type of nucleic acid, including, but not limited to DNA and RNA. For example, in one embodiment, the composition comprises an isolated DNA molecule, including for example, an isolated cDNA molecule, encoding the variant or fragment of a tRNA molecule of the invention. In one embodiment, the composition comprises an isolated RNA molecule.

The nucleic acid molecules of the present invention can be modified to improve stability in serum or in growth medium for cell cultures. Modifications can be added to enhance stability, functionality, and/or specificity and to modulate immunostimulatory properties of the nucleic acid molecule of the invention. For example, in order to enhance the stability, the 3′-residues may be stabilized against degradation.

In one embodiment of the present invention the nucleic acid molecule may contain at least one modified nucleotide analogue. For example, the ends may be stabilized by incorporating modified nucleotide analogues. Non-limiting examples of nucleotide analogues include sugar- and/or backbone-modified ribonucleotides (i.e., include modifications to the phosphate-sugar backbone). For example, the phosphodiester linkages of natural RNA may be modified to include at least one of a nitrogen or sulfur heteroatom. In some backbone-modified ribonucleotides the phosphoester group connecting to adjacent ribonucleotides is replaced by a modified group, e.g., of phosphothioate group. In some sugar-modified ribonucleotides, the 2′ OH-group is replaced by a group selected from H, OR, R, halo, SH, SR, NH₂, NHR, NR₂ or ON, wherein R is C₁-C₆ alkyl, alkenyl or alkynyl and halo is F, Cl, Br or I.

Other examples of modifications are nucleobase-modified ribonucleotides, i.e., ribonucleotides, containing at least one non-naturally occurring nucleobase instead of a naturally occurring nucleobase. Bases may be modified to block the activity of adenosine deaminase. Exemplary modified nucleobases include, but are not limited to, uridine and/or cytidine modified at the 5-position, e.g., 5-(2-amino)propyl uridine, 5-bromo uridine; adenosine and/or guanosines modified at the 8 position, e.g., 8-bromo guanosine; deaza nucleotides, e.g., 7-deaza-adenosine; O- and N-alkylated nucleotides, e.g., N6-methyl adenosine are suitable. It should be noted that the above modifications may be combined.

In some embodiments, the nucleic acid molecule comprises at least one of the following chemical modifications: 2′-H, 2′-O-methyl, or 2′-OH modification of one or more nucleotides. In certain embodiments, a nucleic acid molecule of the invention can have enhanced resistance to nucleases. For increased nuclease resistance, a nucleic acid molecule, can include, for example, 2′-modified ribose units and/or phosphorothioate linkages. For example, the 2′ hydroxyl group (OH) can be modified or replaced with a number of different “oxy” or “deoxy” substituents. For increased nuclease resistance the nucleic acid molecules of the invention can include 2′-O-methyl, 2′-fluorine, 2′-O-methoxyethyl, 2′-O-aminopropyl, 2′-amino, and/or phosphorothioate linkages. Inclusion of locked nucleic acids (LNA), ethylene nucleic acids (ENA), e.g., 2′-4′-ethylene-bridged nucleic acids, and certain nucleobase modifications such as 2-amino-A, 2-thio (e.g., 2-thio-U), G-clamp modifications, can also increase binding affinity to a target.

In one embodiment, the nucleic acid molecule includes a 2′-modified nucleotide, e.g., a 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA). In one embodiment, the nucleic acid molecule includes at least one 2′-O-methyl-modified nucleotide, and in some embodiments, all of the nucleotides of the nucleic acid molecule include a 2′-O-methyl modification.

Nucleic acid agents discussed herein include otherwise unmodified RNA and DNA as well as RNA and DNA that have been modified, e.g., to improve efficacy, and polymers of nucleoside surrogates. Unmodified RNA refers to a molecule in which the components of the nucleic acid, namely sugars, bases, and phosphate moieties, are the same or essentially the same as that which occur in nature, for example, as occur naturally in the human body. The art has referred to rare or unusual, but naturally occurring, RNAs as modified RNAs, see, e.g., Limbach et al. (Nucleic Acids Res., 1994, 22:2183-2196). Such rare or unusual RNAs, often termed modified RNAs, are typically the result of a post-transcriptional modification and are within the term unmodified RNA as used herein. Modified RNA, as used herein, refers to a molecule in which one or more of the components of the nucleic acid, namely sugars, bases, and phosphate moieties, are different from that which occur in nature, for example, different from that which occurs in the human body. While they are referred to as “modified RNAs” they will of course, because of the modification, include molecules that are not, strictly speaking, RNAs. Nucleoside surrogates are molecules in which the ribophosphate backbone is replaced with a non-ribophosphate construct that allows the bases to be presented in the correct spatial relationship such that hybridization is substantially similar to what is seen with a ribophosphate backbone, e.g., non-charged mimics of the ribophosphate backbone.

Modifications of the nucleic acid of the invention may be present at one or more of, a phosphate group, a sugar group, backbone, N-terminus, C-terminus, or nucleobase.

The present invention also includes a vector in which the isolated nucleic acid of the present invention is inserted. The art is replete with suitable vectors that are useful in the present invention.

In some embodiments, the expression of natural or synthetic nucleic acids encoding a variant or fragment of a tRNA molecule is typically achieved by incorporating a nucleic acid encoding the variant or fragment of a tRNA molecule into an appropriate vector for transcription of the variant or fragment of a tRNA molecule. The vectors to be used are suitable for replication and, optionally, integration in eukaryotic cells.

The vectors of the present invention may also be used for nucleic acid immunization using standard gene delivery protocols. Methods for gene delivery are known in the art. See, e.g., U.S. Pat. Nos. 5,399,346, 5,580,859, 5,589,466, incorporated by reference herein in their entireties.

The isolated nucleic acid of the invention can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the vector may be provided to a cell in the form of a viral vector. Viral vector technology is well known in the art and is described, for example, in Sambrook et al. (2012, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used.

For example, vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity. In one embodiment, the composition includes a vector derived from an adeno-associated virus (AAV). Adeno-associated viral (AAV) vectors have become powerful gene delivery tools for the treatment of various disorders. AAV vectors possess a number of features that render them ideally suited for gene therapy, including a lack of pathogenicity, minimal immunogenicity, and the ability to transduce postmitotic cells in a stable and efficient manner. Expression of a particular gene contained within an AAV vector can be specifically targeted to one or more types of cells by choosing the appropriate combination of AAV serotype, promoter, and delivery method

In certain embodiments, the vector also includes conventional control elements which are operably linked to the transgene in a manner which permits its transcription, translation and/or expression in a cell transfected with the plasmid vector or infected with the virus produced by the invention. As used herein, “operably linked” sequences include both expression control sequences that are contiguous with the gene of interest and expression control sequences that act in trans or at a distance to control the gene of interest. Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A great number of expression control sequences, including promoters which are native, constitutive, inducible and/or tissue-specific, are known in the art and may be utilized.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EF-1α). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

Enhancer sequences found on a vector also regulates expression of the gene contained therein. Typically, enhancers are bound with protein factors to enhance the transcription of a gene. Enhancers may be located upstream or downstream of the gene it regulates. Enhancers may also be tissue-specific to enhance transcription in a specific cell or tissue type. In one embodiment, the vector of the present invention comprises one or more enhancers to boost transcription of the gene present within the vector.

In order to assess the expression of a variant or fragment of a tRNA molecule of the invention, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2012, Molecular Cloning: A Laboratory Manual, Cold Spring Harbor Laboratory, New York). In one embodiment, the method of introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, MO; dicetyl phosphate (“DCP”) can be obtained from K & K Laboratories (Plainview, NY); cholesterol (“Choi”) can be obtained from Calbiochem-Behring; dimyristyl phosphatidylglycerol (“DMPG”) and other lipids may be obtained from Avanti Polar Lipids, Inc. (Birmingham, AL). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

In one embodiment, the present invention provides a delivery vehicle comprising a variant or fragment of a tRNA molecule of the invention, or a nucleic acid molecule encoding a variant or fragment of a tRNA molecule of the invention. Exemplary delivery vehicles include, but are not limited to, microspheres, microparticles, nanoparticles, polymerosomes, liposomes, and micelles. For example, in certain embodiments, the delivery vehicle is loaded with a variant or fragment of a tRNA molecule of the invention, or a nucleic acid molecule encoding a variant or fragment of a tRNA molecule of the invention. In certain embodiments, the delivery vehicle provides for controlled release, delayed release, or continual release of its loaded cargo. In certain embodiments, the delivery vehicle comprises a targeting moiety that targets the delivery vehicle to a treatment site.

### Pharmaceutical Compositions and Administration

Compositions comprising a variant or fragment of a tRNA molecule of the invention, as described elsewhere herein, can be formulated and administered to a subject. Therefore, the invention encompasses the preparation and use of pharmaceutical compositions comprising a variant or fragment of a tRNA molecule useful for the treatment or prevention of a disease or disorder as an active ingredient. Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

As used herein, the term “pharmaceutically-acceptable carrier” means a chemical composition with which an appropriate variant or fragment of a tRNA molecule, may be combined and which, following the combination, can be used to administer the composition to a subject.

In some embodiments, pharmaceutical compositions can include large, slowly metabolized macromolecules such as proteins, polysaccharides such as chitosan, polylactic acids, polyglycolic acids and copolymers (such as latex functionalized Sepharose™, agarose, cellulose, and the like), polymeric amino acids, amino acid copolymers, and lipid aggregates (such as oil droplets or liposomes).

The pharmaceutical compositions useful for practicing the invention may be administered to deliver a dose of between about 0.1 ng/kg/day and 100 mg/kg/day, or more.

In various embodiments, the pharmaceutical compositions useful in the methods of the invention may be administered, by way of example, systemically, parenterally, or topically, such as, in oral formulations, inhaled formulations, including solid or aerosol, and by topical or other similar formulations. In addition to the appropriate therapeutic composition, such pharmaceutical compositions may contain pharmaceutically acceptable carriers and other ingredients known to enhance and facilitate drug administration. Other possible formulations, such as nanoparticles, liposomes, other preparations containing the active ingredient, and immunologically based systems may also be used to administer an appropriate modulator thereof, according to the methods of the invention.

A carrier may bear a subject agent (e.g., a variant or fragment of a tRNA molecule) in a variety of ways, including covalent bonding either directly or via a linker group, and non-covalent associations. The nature of the carrier can be either soluble or insoluble for purposes of the invention.

Acceptable carriers, excipients, or stabilizers are non-toxic to recipients at the dosages and concentrations employed, and include buffers such as phosphate, citrate, and other organic acids; antioxidants including ascorbic acid and methionine; preservatives (such as octadecyidimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS' or polyethylene glycol (PEG). Formulations to be used for in vivo administration must be sterile. This is readily accomplished by filtration through sterile filtration membranes.

The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Compositions can be prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above. Langer, Science 249: 1527, 1990 and Hanes, Advanced Drug Delivery Reviews 28: 97-119, 1997. The agents of this invention can be administered in the form of a depot injection or implant preparation which can be formulated in such a manner as to permit a sustained or pulsatile release of the active ingredient. The pharmaceutical compositions are generally formulated as sterile, substantially isotonic and in full compliance with all Good Manufacturing Practice (GMP) regulations of the U.S. Food and Drug Administration.

As used herein, the term “physiologically acceptable” ester or salt means an ester or salt form of the active ingredient which is compatible with any other ingredients of the pharmaceutical composition, which is not deleterious to the subject to which the composition is to be administered.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation.

Pharmaceutical compositions that are useful in the methods of the invention may be prepared, packaged, or sold in formulations suitable for oral, rectal, vaginal, parenteral, topical, pulmonary, intranasal, buccal, intravenous, transdermal, intralesional, subcutaneous, intramuscular, ophthalmic, intrathecal and other known routes of administration. Other contemplated formulations include projected nanoparticles, liposomal preparations, other preparations containing the active ingredient, and immunologically-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents.

Controlled- or sustained-release formulations of a pharmaceutical composition of the invention may be made using conventional technology.

A formulation of a pharmaceutical composition of the invention suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

Pharmaceutically acceptable excipients used in the manufacture of pharmaceutical compositions include, but are not limited to, inert diluents, granulating and disintegrating agents, binding agents, and lubricating agents. Known dispersing agents include, but are not limited to, potato starch and sodium starch glycollate. Known surface active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Liquid formulations of a pharmaceutical composition of the invention may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent.

Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, and hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g. polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin and acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, cutaneous, subcutaneous, intraperitoneal, intravenous, intramuscular, intracisternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In some embodiments of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

Formulations suitable for topical administration include, but are not limited to, liquid or semi-liquid preparations such as liniments, lotions, oil-in-water or water-in-oil emulsions such as creams, ointments or pastes, and solutions or suspensions. Topically-administrable formulations may, for example, comprise from about 1% to about 10% (w/w) active ingredient, although the concentration of the active ingredient may be as high as the solubility limit of the active ingredient in the solvent Formulations for topical administration may further comprise one or more of the additional ingredients described herein.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Pharmaceutical compositions of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration preferably have an average diameter in the range from about 0.1 to about 200 nanometers. The formulations described herein as being useful for pulmonary delivery are also useful for intranasal delivery of a pharmaceutical composition of the invention. Another formulation suitable for intranasal administration is a coarse powder comprising the active ingredient and having an average particle from about 0.2 to 500 micrometers.

Such a formulation is administered in the manner in which snuff is taken i.e. by rapid inhalation through the nasal passage from a container of the powder held close to the nares. Formulations suitable for nasal administration may, for example, comprise from about as little as 0.1% (w/w) and as much as 100% (w/w) of the active ingredient, and may further comprise one or more of the additional ingredients described herein.

A pharmaceutical composition of the invention may be prepared, packaged, or sold in a formulation suitable for buccal administration. Such formulations may, for example, be in the form of tablets or lozenges made using conventional methods, and may, for example, contain 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers, and may further comprise one or more of the additional ingredients described herein.

A pharmaceutical composition of the invention may be prepared, packaged, or sold in a formulation suitable for ophthalmic administration. Such formulations may, for example, be in the form of eye drops including, for example, a 0.1-1.0% (w/w) solution or suspension of the active ingredient in an aqueous or oily liquid carrier. Such drops may further comprise buffering agents, salts, or one or more other of the additional ingredients described herein. Other ophthalmically-administrable formulations which are useful include those which comprise the active ingredient in microcrystalline form or in a liposomal preparation.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed., 1985, Remington's Pharmaceutical Sciences, Mack Publishing Co., Easton, Pa., which is incorporated herein by reference.

Typically dosages of the compound of the invention which may be administered to an animal, preferably a human, range in amount from about 0.001 mg to about 1000 mg per kilogram of body weight of the animal. The precise dosage administered will vary depending upon any number of factors, including, but not limited to, the type of animal and type of disease or disorder being treated, the age of the animal and the route of administration. Preferably, the dosage of the compound will vary from about 0.1 mg to about 10 mg per kilogram of body weight of the animal. The compound can be administered to an animal as frequently as several times daily, or it can be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease or disorder being treated, the type and age of the animal, etc.

### Methods of Treatment and Prevention

In various embodiments, the present invention includes compositions comprising a fragment of a tRNA molecule, or a variant thereof, and methods of using the same for increasing the immune response in a cell, tissue, organ, system, or subject in need thereof. Therefore, in one embodiment, the invention provides methods of administering a composition comprising a fragment of a tRNA molecule, or a variant thereof, to a subject in need thereof.

In various embodiments, the present invention includes compositions comprising a TLR agonist, and methods of using the same for increasing TLR activity, such as signaling through at least one TLR, in a cell, tissue, organ, system, or subject in need thereof. In various embodiments, the TLR agonist compositions, and methods of treatment of the invention, increase the amount of TLR activity, the amount of immune cell activity, or both. Therefore, in one embodiment, the invention provides methods of administering a composition comprising a TLR agonist to a subject in need thereof.

In one embodiment, the compositions of the invention can be used for the treatment or prevention of a disease or disorder in a subject in need thereof. In various embodiments, the compositions of the invention can be used to increase at least one of TLR activity, an immune response, or a combination thereof in a subject, and thus improve therapeutic outcomes to a disease or disorder. As such, in one embodiment, the compositions of the invention can be used as an adjuvant to increase an immune response in a subject in need thereof.

Diseases and disorders that can be treated using the compositions of the invention include, but are not limited to, cancer and infectious diseases.

The following are non-limiting examples of cancers that can be treated or prevented by the methods and compositions of the invention: acute lymphoblastic leukemia, acute myeloid leukemia, adrenocortical carcinoma, appendix cancer, basal cell carcinoma, bile duct cancer, bladder cancer, bone cancer, brain and spinal cord tumors, brain stem glioma, brain tumor, breast cancer, bronchial tumors, burkitt lymphoma, carcinoid tumor, central nervous system atypical teratoid/rhabdoid tumor, central nervous system embryonal tumors, central nervous system lymphoma, cerebellar astrocytoma, cerebral astrocytoma/malignant glioma, cerebral astrocytotna/malignant glioma, cervical cancer, childhood visual pathway tumor, chordoma, chronic lymphocytic leukemia, chronic myelogenous leukemia, chronic myeloproliferative disorders, colon cancer, colorectal cancer, craniopharyngioma, cutaneous cancer, cutaneous t-cell lymphoma, endometrial cancer, ependymoblastoma, ependymoma, esophageal cancer, ewing family of tumors, extracranial cancer, extragonadal germ cell tumor, extrahepatic bile duct cancer, extrahepatic cancer, eye cancer, fungoides, gallbladder cancer, gastric (stomach) cancer, gastrointestinal cancer, gastrointestinal carcinoid tumor, gastrointestinal stromal tumor (gist), germ cell tumor, gestational cancer, gestational trophoblastic tumor, glioblastoma, glioma, hairy cell leukemia, head and neck cancer, hepatocellular (liver) cancer, histiocytosis, hodgkin lymphoma, hypopharyngeal cancer, hypothalamic and visual pathway glioma, hypothalamic tumor, intraocular (eye) cancer, intraocular melanoma, islet cell tumors, kaposi sarcoma, kidney (renal cell) cancer, langerhans cell cancer, langerhans cell histiocytosis, laryngeal cancer, leukemia, lip and oral cavity cancer, liver cancer, lung cancer, lymphoma, macroglobulinemia, malignant fibrous histiocvtoma of bone and osteosarcoma, medulloblastoma, medulloepithelioma, melanoma, merkel cell carcinoma, mesothelioma, metastatic squamous neck cancer with occult primary, mouth cancer, multiple endocrine neoplasia syndrome, multiple myeloma, mycosis, myelodysplastic syndromes, myelodysplastic/myeloproliferative diseases, myelogenous leukemia, myeloid leukemia, myeloma, myeloproliferative disorders, nasal cavity and paranasal sinus cancer, nasopharyngeal cancer, neuroblastoma, non-hodgkin lymphoma, non-small cell lung cancer, oral cancer, oral cavity cancer, oropharyngeal cancer, osteosarcoma and malignant fibrous histiocytoma, osteosarcoma and malignant fibrous histiocytoma of bone, ovarian, ovarian cancer, ovarian epithelial cancer, ovarian germ cell tumor, ovarian low malignant potential tumor, pancreatic cancer, papillomatosis, paraganglioma, parathyroid cancer, penile cancer, pharyngeal cancer, pheochromocytoma, pineal parenchymal tumors of intermediate differentiation, pineoblastoma and supratentorial primitive neuroectodermal tumors, pituitary tumor, plasma cell neoplasm, plasma cell neoplasm/multiple myeloma, pleuropulmonary blastoma, primary central nervous system cancer, primary central nervous system lymphoma, prostate cancer, rectal cancer, renal cell (kidney) cancer, renal pelvis and ureter cancer, respiratory tract carcinoma involving the nut gene on chromosome 15, retinoblastoma, rhabdomyosarcoma, salivary gland cancer, sarcoma, sezary syndrome, skin cancer (melanoma), skin cancer (nonmelanoma), skin carcinoma, small cell lung cancer, small intestine cancer, soft tissue cancer, soft tissue sarcoma, squamous cell carcinoma, squamous neck cancer, stomach (gastric) cancer, supratentorial primitive neuroectodermal tumors, supratentorial primitive neuroectodermal tumors and pineoblastoma, T-cell lymphoma, testicular cancer, throat cancer, thymoma and thymic carcinoma, thyroid cancer, transitional cell cancer, transitional cell cancer of the renal pelvis and ureter, trophoblastic tumor, urethral cancer, uterine cancer, uterine sarcoma, vaginal cancer, visual pathway and hypothalamic glioma, vulvar cancer, waldenstrom macroglobulinemia, and Wilms Tumor.

Thus, non-limiting examples of cancers that can be treated or prevented by the methods and compositions of the disclosure include solid tumor cancers, liquid cancers, blood cancers, teratomas, sarcomas, and carcinomas.

### Co-Administration

In some embodiments, a variant tRNA molecule or tRNA molecule fragment of the invention is administered in combination with at least one additional agent. The terms “co-administration”, “co-administer”, and “in combination with” include the administration of two or more therapeutic agents (e.g., a variant tRNA molecule or tRNA molecule fragment such as a 5′-tRNA half in combination with an additional agent) either simultaneously, concurrently or sequentially within no specific time limits. In some embodiments, the agents are present in the cell or in the subject's body at the same time or exert their biological or therapeutic effect at the same time. In some embodiments, the therapeutic agents are in the same composition or unit dosage form. In other embodiments, the therapeutic agents are in separate compositions or unit dosage forms. In certain embodiments, a first agent can be administered prior to (e.g., minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent.

In some embodiments, a variant tRNA molecule or tRNA molecule fragment of the invention is co-administered with an immunotherapeutic drug, therapeutic drug to treat an infection, or a cancer therapeutic. Such administration may involve concurrent (i.e. at the same time), prior, or subsequent administration of the drug/antibody with respect to the administration of an agent or agents of the disclosure. A person of ordinary skill in the art would have no difficulty determining the appropriate timing, sequence and dosages of administration for particular drugs and compositions of the present disclosure. Exemplary cancer therapeutics that can be co-administered with the variant tRNA molecule or tRNA molecule fragment of the invention include, but are not limited to antibodies selective for tumor cell markers, radiation, surgery, and/or hormone deprivation.

In some embodiments, treatment is accomplished by administering a combination (co-administration) of a variant tRNA molecule or tRNA molecule fragment of the invention with another agent (e.g., an immune stimulant, an agent to treat chronic infection, a cytotoxic agent, an anti-cancer agent, etc.).

Other anti-cancer agents that can be used in combination with the disclosed compounds include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride. Other anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer. In one embodiment, the anti-cancer drug is 5-fluorouracil, taxol, or leucovorin.

The compositions and methods of the present invention can be used in combination with other treatment regimens, including virostatic and virotoxic agents, antibiotic agents, antifungal agents, anti-inflammatory agents (steroidal and non-steroidal), antidepressants, anxiolytics, pain management agents, (acetaminophen, aspirin, ibuprofen, opiates (including morphine, hydrocodone, codeine, fentanyl, methadone)), steroids (including prednisone and dexamethasone), and antidepressants (including gabapentin, amitriptyline, imipramine, doxepin) antihistamines, antitussives, muscle relaxants, bronchodilators, beta-agonists, anticholinergics, corticosteroids, mast cell stabilizers, leukotriene modifiers, methylxanthines, as well as combination therapies, and the like. The invention can also be used in combination with other treatment modalities, such as chemotherapy, cryotherapy, hyperthermia, radiation therapy, and the like.

### Pharmaceutical Formulations

The therapeutic and prophylactic methods of the invention thus encompass the use of pharmaceutical compositions comprising one or more tRNA variant molecule or fragment of a tRNA molecule, or combinations thereof, as described herein. The pharmaceutical compositions useful for practicing the invention may be administered to deliver a dose of between 1 ng/kg/day and 100 mg/kg/day. In one embodiment, the invention envisions administration of a dose which results in a concentration of the compound of the present invention between 1 μM and 10 μM in a mammal.

The pharmaceutical compositions useful for practicing the invention may be administered to deliver a dose of at least about 1 ng/kg, at least about 5 ng/kg, at least about 10 ng/kg, at least about 25 ng/kg, at least about 50 ng/kg, at least about 100 ng/kg, at least about 500 ng/kg, at least about 1 μg/kg, at least about 5 μg/kg, at least about 10 μg/kg, at least about 25 μg/kg, at least about 50 μg/kg, at least about 100 μg/kg, at least about 500 μg/kg, at least about 1 mg/kg, at least about 5 mg/kg, at least about 10 mg/kg, at least about 25 mg/kg, at least about 50 mg/kg, at least about 100 mg/kg, at least about 200 mg/kg, at least about 300 mg/kg, at least about 400 mg/kg, and at least about 500 mg/kg of body weight of the subject. In one embodiment, the invention administers a dose which results in a concentration of the tRNA variant molecule or fragment of a tRNA molecule of the present invention of at least about 1 μM, at least about 10 μM, at least about 100 μM, at least about 1 nM, at least about 10 nM, at least about 100 nM, at least about 1 μM, at least about 2 μM, at least about 3 μM, at least about 4 μM, at least about 5 μM, at least about 6 μM, at least about 7 μM, at least about 8 μM, at least about 9 μM and at least about 10 μM in an individual. In another embodiment, the invention envisions administration of a dose which results in a concentration of the anti-YKL-40 antibody of the present invention between at least about 1 μM, at least about 10 μM, at least about 100 μM, at least about 1 nM, at least about 10 nM, at least about 100 nM, at least about 1 μM, at least about 2 μM, at least about 3 μM, at least about 4 μM, at least about 5 μM, at least about 6 μM, at least about 7 μM, at least about 8 μM, at least about 9 μM and at least about 10 μM in the plasma of an individual.

In some embodiments, the pharmaceutical compositions useful for practicing the invention may be administered to deliver a dose of no more than about 1 ng/kg, no more than about 5 ng/kg, no more than about 10 ng/kg, no more than about 25 ng/kg, no more than about 50 ng/kg, no more than about 100 ng/kg, no more than about 500 ng/kg, no more than about 1 μg/kg, no more than about 5 μg/kg, no more than about 10 μg/kg, no more than about 25 μg/kg, no more than about 50 μg/kg, no more than about 100 μg/kg, no more than about 500 μg/kg, no more than about 1 mg/kg, no more than about 5 mg/kg, no more than about 10 mg/kg, no more than about 25 mg/kg, no more than about 50 mg/kg, no more than about 100 mg/kg, no more than about 200 mg/kg, no more than about 300 mg/kg, no more than about 400 mg/kg, and no more than about 500 mg/kg of body weight of the subject. In one embodiment, the invention administers a dose which results in a concentration of the tRNA variant molecule or fragment of a tRNA molecule of the present invention of no more than about 1 μM, no more than about 10 μM, no more than about 100 μM, no more than about 1 nM, no more than about 10 nM, no more than about 100 nM, no more than about 1 μM, no more than about 2 μM, no more than about 3 μM, no more than about 4 μM, no more than about 5 μM, no more than about 6 μM, no more than about 7 μM, no more than about 8 μM, no more than about 9 μM and no more than about 10 μM in an individual. In another embodiment, the invention envisions administration of a dose which results in a concentration of the tRNA variant molecule or fragment of a tRNA molecule of the present invention between no more than about 1 μM, no more than about 10 μM, no more than about 100 μM, no more than about 1 nM, no more than about 10 nM, no more than about 100 nM, no more than about 1 μM, no more than about 2 μM, no more than about 3 μM, no more than about 4 μM, no more than about 5 μM, no more than about 6 μM, no more than about 7 μM, no more than about 8 μM, no more than about 9 μM and no more than about 10 μM in the plasma of an individual. Also contemplated are dosage ranges between any of the doses disclosed herein.

Typically, dosages which may be administered in a method of the invention to an animal, n some embodiments a human, range in amount from 0.5 μg to about 50 mg per kilogram of body weight of the animal. While the precise dosage administered will vary depending upon any number of factors, including but not limited to, the type of animal and type of disease state being treated, the age of the animal and the route of administration. In some embodiments, the dosage of the compound will vary from about 1 μg to about 10 mg per kilogram of body weight of the animal. In some embodiments, the dosage will vary from about 3 μg to about 1 mg per kilogram of body weight of the animal.

The composition of the invention may be administered to subject as frequently as several times daily, or it may be administered less frequently, such as once a day, twice a day, thrice a day, once a week, twice a week, thrice a week, once every two weeks, twice every two weeks, thrice every two weeks, once a month, twice a month, thrice a month, or even less frequently, such as once every several months or even once or a few times a year or less. The frequency of the dose will be readily apparent to the skilled artisan and will depend upon any number of factors, such as, but not limited to, the type and severity of the disease being treated, the type and age of the animal, etc. The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the description of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as non-human primates, cattle, pigs, horses, sheep, cats, and dogs.

Pharmaceutical compositions that are useful in the methods of the invention may be prepared, packaged, or sold in formulations suitable for ophthalmic, oral, rectal, vaginal, parenteral, topical, pulmonary, intranasal, buccal, or another route of administration. Other contemplated formulations include projected nanoparticles, liposomal preparations, resealed erythrocytes containing the active ingredient, and immunologically-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient (e.g., tRNA variant molecule or fragment of a tRNA molecule), the pharmaceutically acceptable carrier, and any additional ingredients in a pharmaceutical composition of the invention will vary, depending upon the identity, size, and condition of the individual treated and further depending upon the route by which the composition is to be administered. By way of example, the composition may comprise between 0.1% and 100% (w/w) active ingredient. In various embodiments, the composition comprises at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, at least about 40%, at least about 41%, at least about 42%, at least about 43%, at least about 44%, at least about 45%, at least about 46%, at least about 47%, at least about 48%, at least about 49%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% (w/w) active ingredient.

In addition to the active ingredient, a pharmaceutical composition of the invention may further comprise one or more additional pharmaceutically active agents. Other active agents useful in the treatment of fibrosis include anti-inflammatories, including corticosteroids, and immunosuppressants.

Controlled- or sustained-release formulations of a pharmaceutical composition of the invention may be made using conventional technology.

Parenteral administration of a pharmaceutical composition includes any route of administration characterized by physical breaching of a tissue of an individual and administration of the pharmaceutical composition through the breach in the tissue. Parental administration can be local, regional or systemic. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intravenous, intraocular, intravitreal, subcutaneous, intraperitoneal, intramuscular, intradermal, intrasternal injection, and intratumoral.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. In some embodiments, such powders comprise particles wherein at least 98% of the particles by weight have a diameter greater than 0.5 nanometers and at least 95% of the particles by number have a diameter less than 7 nanometers. In some embodiments, at least 95% of the particles by weight have a diameter greater than 1 nanometer and at least 90% of the particles by number have a diameter less than 6 nanometers. In some embodiments, dry powder compositions include a solid fine powder diluent such as sugar and are conveniently provided in a unit dose form.

Low boiling propellants generally include liquid propellants having a boiling point of below 65° F. at atmospheric pressure. Generally, the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (in some embodiments having a particle size of the same order as particles comprising the active ingredient).

Pharmaceutical compositions of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. In some embodiments, the droplets provided by this route of administration have an average diameter in the range from about 0.1 to about 200 nanometers.

The formulations described herein as being useful for pulmonary delivery are also useful for intranasal delivery of a pharmaceutical composition of the invention.

Another formulation suitable for intranasal administration is a coarse powder comprising the active ingredient and having an average particle from about 0.2 to 500 micrometers. Such a formulation is administered in the manner in which snuff is taken i.e. by rapid inhalation through the nasal passage from a container of the powder held close to the nares.

Formulations suitable for nasal administration may, for example, comprise from about as little as 0.1% (w/w) and as much as 100% (w/w) of the active ingredient, and may further comprise one or more of the additional ingredients described herein.

A pharmaceutical composition of the invention may be prepared, packaged, or sold in a formulation suitable for buccal administration. Such formulations may, for example, be in the form of tablets or lozenges made using conventional methods, and may, for example, 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. In some embodiments, such powdered, aerosolized, or aerosolized formulations, when dispersed, have an average particle or droplet size in the range from about 0.1 to about 200 nanometers, and may further comprise one or more of the additional ingredients described herein.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Remington's Pharmaceutical Sciences (1985, Genaro, ed., Mack Publishing Co., Easton, PA), which is incorporated herein by reference.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

### Example 1: Infection-Induced 5′-tRNA^{HisGUG} Half Molecules Activate Toll-Like Receptor 7

Although the expression of tRNA halves is regulated by various biological factors, such as stresses and sex hormones (Fu H, et al., 2009, FEBS Lett, 583: 437-442; Yamasaki S, et al., 2009, J Cell Biol, 185: 35-42; Honda S, et al., 2015, Proc Natl Acad Sci USA, 112: E3816-3825), how bacterial infection regulates their expression is not fully understood. Herein is described the expressional regulation and functional involvement of 5′-tRNA half molecules in the infection-induced innate immune response. Infection of Mycobacterium bovis bacillus Calmette-Guerin (BCG) and surface TLR activation induced the expression of 5′-tRNA halves in human monocyte-derived macrophages (HMDMs). cP-RNA-seq-based identification of the induced 5′-tRNA halves in HMDMs and their secreted EVs revealed selective and abundant packaging of 5′-tRNA halves into EVs. Further, the delivery of the EV-5′-tRNA halves into endosomes of recipient cells and strong TLR7 activation by 5′-tRNA halves was experimentally demonstrated. Induction of the expression and secretion of 5′-tRNA halves was further confirmed in the plasma of Mtb-infected patients, verifying that the observed phenomena occur not only in cell culture systems but also in actual pathological situations. This Example unveils a novel tRNA-engaged pathway in the innate immune response and newly assigned the role of immune activators to 5′-tRNA halves.

The methods of the present Example are now described herein.

Cell Culture, BCG Infection, PAMP Treatment, and NF-κB Inhibition THP-1 human acute monocytic leukemia cells (American Type Culture Collection) were cultured in RPMI 1640 medium (Corning) and differentiated into HMDMs using phorbol 12-myristate 13-acetate (PMA; Sigma-Aldrich) as described previously (Pawar K, et al., 2016, Front Cell Infect Microbiol, 6: 27; Pawar K, et al., 2016, Sci Rep, 6: 19416). Human CD14+ monocytes (Precision for Medicine) were cultured in Gibco SFM medium (Thermo Fisher Scientific) and differentiated into PHMDMs using macrophage colony-stimulating factor (M-CSF; Tonbo Biosciences) as described previously (Zur Bruegge J, et al., 2016, Eur J Microbiollmmunol, 6: 99-108). HMDMs were infected with viable or heat-killed (HK) M. bovis BCG (DSMZ) as described previously ((Pawar K, et al., 2016, Front Cell Infect Microbiol, 6: 27; Pawar K, et al., 2016, Sci Rep, 6: 19416). Zero viability of HK-BCG was confirmed by spading its suspension on Middlebrook 7H11 agar plates supplemented with OADC and confirming the absence of colonies in at least 3 weeks. For activation of surface TLRs, HMDMs and PHMDMs were cultured with medium containing 100 ng/ml of LPS from E. coli O111:B4 (Sigma-Aldrich) or PGN from B. subtilis (Sigma-Aldrich) for 12 h. For inhibition of NF-κB, HMDMs were treated with 40 μM of JSH-23 (Sigma-Aldrich) for 24 h.

EV Isolation

EVs were isolated from the culture medium of LPS-treated HMDMs according to an ultracentrifugation-based method described previously (Zhang Y, et al., 2010, Mol Cell, 39: 133-144). In brief, dead cells and cell debris in the culture medium were removed by successive centrifugation at 300 g for 10 min, 2000 g for 10 min, and 10,000 g for 30 min. The supernatant was then ultracentrifuged using Sorvall WX+ Ultracentrifuge Series (Thermo Fisher Scientific) at 110,000 g for 2 h. The pellet was washed with PBS and ultracentrifuged again at 110,000 g for 2 hours to eliminate contaminant proteins. The final pellet was collected as the EV fraction. The data regarding EV isolation and characterization is available in EV-TRACK database (EV-TRACK ID: EV190062) (Van Deun J, et al., 2017, Nat Methods, 14: 228-232). To confirm the presence of EV-RNAs, the isolated EVs were incubated with PureLink RNase A (200 ng/∝l, Thermo Fisher Scientific) with or without 0.1% Triton X-100 at 37° C. for 30 min.

NTA and Transmission Electron Microscopy

Size distributions of the isolated EVs were analyzed by NTA using NanoSight NS300 (Malvern Analytical), as described previously (Krishn S R, et al., 2019, Matrix Biology, 77: 41-57), at the Flow Cytometry Facility of the Sidney Kimmel Cancer Center at Thomas Jefferson University. The isolated EVs were further visualized by transmission electron microscopy (JEOL) at the Centralized Research Facilities at Drexel University.

Quantification of RNAs by TaqMan RT-qPCR, Stem-Loop RT-qPCR, and Standard RT-qPCR

Total RNA from the cells and EVs was isolated using TRIsure (Bioline). TaqMan RT-qPCR for specific quantification of 5′-tRNA halves was performed according to a previously-described tRNA half quantification method (Honda S, et al., 2015, Proc Natl Acad Sci USA, 112: E3816-3825). Briefly, to remove cP from 5′-tRNA halves, total RNA was treated with T4 PNK, followed by ligation to a 3′-RNA adapter by T4 RNA ligase. Ligated RNA was then subjected to TaqMan RT-qPCR using the One Step PrimeScript RT-PCR Kit (Takara Bio), 200 nM of a TaqMan probe targeting the boundary of the target RNA and the 3′-adapter, and forward and reverse primers. The TaqMan probe and primer sequences are shown in Table 1 below. Stem-loop RT-qPCR for quantification of miRNAs and piRNAs was performed as previously described (Honda S, et al., 2017, Sci Rep, 7: 4110; Chen C, et al., 2005, Nucleic Acids Res, 33: e179). In brief, total RNA was treated with DNase I (Promega) and subjected to reverse transcription using SuperScript III reverse transcriptase (Thermo Fisher Scientific) and a stem-loop reverse primer. The synthesized cDNAs were then subjected to PCR using Ssofast Evagreen Supermix (Bio-Rad) and forward and reverse primers. Sequences of the primers used are shown in Table 2 below. Standard RT-qPCR was used for quantification of mRNAs. Briefly, DNase I-treated total RNA was subjected to reverse transcription using RevertAid Reverse Transcriptase (Thermo Fisher Scientific) and a reverse primer. The synthesized cDNAs were then subjected to PCR using 2×qPCR Master Mix (Bioland Scientific) and forward and reverse primers. Sequences of the primers used are shown in Table 3 below.

TABLE 1
Sequences of TaqMan probes and primers for TaqMan RT-qPCR

| Target | Probe/primer | Sequence (5′-3′) |
|---|---|---|
| 5′-tRNA^{HisGUG} half | TaqMan | /5HEX/TAGTACTCT/ZEN/GCGTTGGAACACT |
| | | GCGTTTGC/3IABKFQ/ |
| | Forward | GCTCGCCGTGATCGTATAGT |
| | Reverse | GATCGTCGGACTGTAGAACTC |
| 5′-tRNA^{GluCUC} half | TaqMan | /56FAM/CGCTCGAAC/ZEN/ACTGCGTTTG/ |
| | | 3IABkFQ/ |
| | Forward | TCCCTGGTGGTCTAGTGG |
| | Reverse | GATCGTCGGACTGTAGAACTC |

All synthetic probes and primes used in the present study were synthesized by Integrated DNA Technologies. TaqMan probes contain Hexachlorofluorescein (HEX), 6-carboxyfluorescein (FAM), and ZEN/Iowa Black as the fluorophore and quencher, respectively.

TABLE 2
Sequences of primers for stem-loop RT-qPCR

| Target | Primer | Sequence (5′-3′) |
|---|---|---|
| miR-21 | Stem-loop RT | GTCGTATCCAGTGCAGGGTCCGAGGTATTCG |
| | | CACTGGATACGACTCAACA (SEQ ID NO: 18) |
| | Forward | CGGCGTAGCTTATCAGACT (SEQ ID NO: 19) |
| | Reverse | GTGCAGGGTCCGAGGT (SEQ ID NO: 20) |
| miR-150 | Stem-loop RT | GTCGTATCCAGTGCAGGGTCCGAGGTATTCG |
| | | CACTGGATACGACCACTGG (SEQ ID NO: 21) |
| | Forward | GACGTCTCCCAACCCTTG (SEQ ID NO: 22) |
| | Reverse | GTGCAGGGTCCGAGGT (SEQ ID NO: 23) |
| piR-3 (spike-in) | Stem-loop RT | GTCGTATCCAGTGCAGGGTCCGAGGTATTCG |
| | | CACTGGATACGACACCACT (SEQ ID NO: 24) |
| | Forward | CGGCGTAGCTTATCAGACT (SEQ ID NO: 25) |
| | Reverse | GTGCAGGGTCCGAGGT (SEQ ID NO: 26) |

TABLE 3
Sequences of primers for standard RT-qPCR

| Target | Primer | Sequence (5′-3′) |
|---|---|---|
| MIP-1α | Forward | GCTGTCCTCCTCTGCACCAT (SEQ ID NO: 27) |
| | Reverse | ATCTGCCGGGAGGTGTAGCT (SEQ ID NO: 28) |
| MIP-1ß | Forward | CATGCTAGTAGCTGCCTTCTGC (SEQ ID NO: 29) |
| | Reverse | AGCTTCCTCGCGGTGTAAGA (SEQ ID NO: 30) |
| TNFα | Forward | GAGCACTGAAAGCATGATCC (SEQ ID NO: 31) |
| | Reverse | CGAGAAGATGATCTGACTGCC (SEQ ID NO: 32) |
| ANG | Forward | AGAAGCGGGTGAGAAACAAAAC (SEQ ID NO: 33) |
| | Reverse | AGTGCTGGGTCAGGAAGTGTG (SEQ ID NO: 34) |
| IL-1ß | Forward | CAGGCTGCTCTGGGATTCTC (SEQ ID NO: 35) |
| | Reverse | CCTGGAAGGAGCACTTCATCT (SEQ ID NO: 36) |
| IL-12p40 | Forward | GAGTCTGCCCATTGAGGTCAT (SEQ ID NO: 37) |
| | Reverse | AATTTTCATCCTGGATCAGAACC (SEQ ID NO: 38) |
| TLR7 | Forward | CCTTTCCCAGAGCATACAGC (SEQ ID NO: 39) |
| | Reverse | GGACAGAACTCCCACAGAGC (SEQ ID NO: 40) |
| TLR8 | Forward | CAGAGCATCAACCAAAGCAA (SEQ ID NO: 41) |
| | Reverse | GCTGCCGTAGCCTCAAATAC (SEQ ID NO: 42) |
| RPLP0 | Forward | CTATCATCAACGGGTACAAACGAG (SEQ ID NO: 43) |
| | Reverse | CAGATGGATCAGCCAAGAAGG (SEQ ID NO: 44) |
| GAPDH | Forward | GTCTTCACCACCATGGAGAAGG (SEQ ID NO: 45) |
| | Reverse | ATGATCTTGAGGCTGTTGTCAT (SEQ ID NO: 46) |
| U6 snRNA | Forward | TCGCTTCGGCAGCACATATAC (SEQ ID NO: 47) |
| | Reverse | CGAATTTGCGTGTCATCCTTG (SEQ ID NO: 48) |

Northern Blot

Northern blot was performed with the following antisense probes: 5′-tRNA^{HisGUG} half, 5′-CAGAGTACTAACCACTATACGATCACGGC-3′ (SEQ ID NO:49); 5′-tRNA^{GluCUC} half, 5′-GCGCCGAATCCTAACCACT-3′ (SEQ ID NO:50); and miR-16, 5′-GCCAATATTTACGTGCTGCTA-3′ (SEQ ID NO:51).

Western Blot

Western blot was performed as described previously (Honda S, et al., 2017, Sci Rep, 7: 4110). Lysates of HMDMs or their EVs were prepared in RIPA buffer supplemented with cOmplete Protease Inhibitor Cocktail (Roche). Anti-Alix (1A12, Santa Cruz Biotechnology), anti-CD63 (Santa Cruz Biotechnology), anti-Calnexin (AF18, Santa Cruz Biotechnology), anti-cytochrome c (A-8, Santa Cruz Biotechnology), and anti-TLR7 (4F4, sc-57463, Santa Cruz Biotechnology) were used as primary antibodies.

cP-RNA-Seq and Bioinformatics

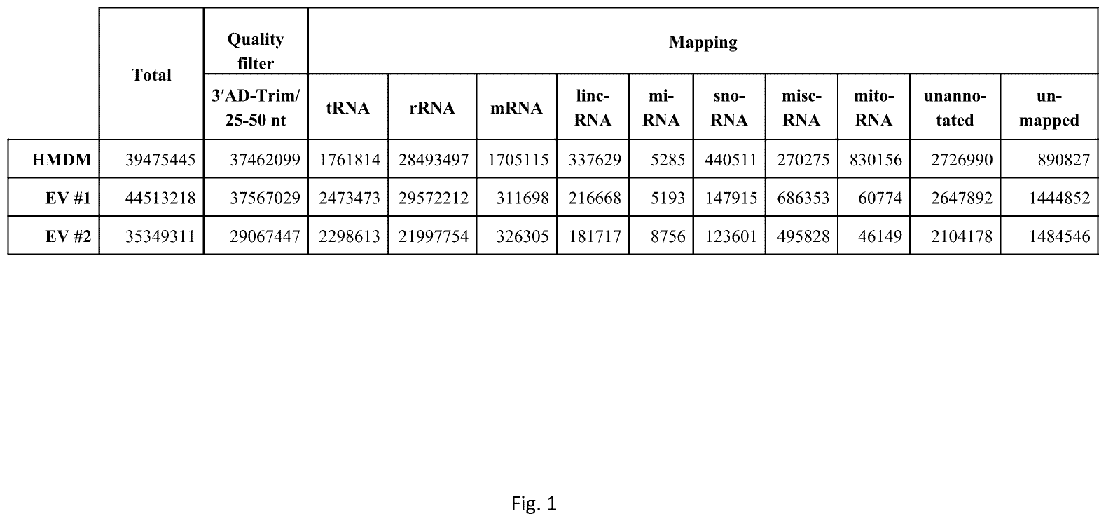

For cP-RNA-seq, 25-50-nt RNAs were gel-purified from the total RNA of LPS-treated HMDMs and subjected to the cP-RNA-seq procedure as previously described (Honda S, et al., 2015, Proc Natl Acad Sci USA, 112: E3816-3825; Honda S, et al., 2017, Nucleic Acids Res, 45: 9108-9120; Honda S, et al., 2016, Nat Protoc, 11: 476-489; Shigematsu M, et al., 2020, RNA Biol, 17: 1060-1069; Shigematsu M, et al., 2019, PLoS Genet, 15: e1008469). For EV-5′-tRNA half sequencing, EV-RNA was first treated with T4 PNK to remove cP from the 5′-tRNA halves, followed by adapter ligation and cDNA amplification using the TruSeq Small RNA Sample Prep Kit (Illumina). The amplified cDNAs were gel-purified and sequenced using the Illumina NextSeq 500 system at the MetaOmics Core Facility of the Sidney Kimmel Cancer Center at Thomas Jefferson University. The sequence libraries contain ˜35-44 million raw reads (FIG. 1) and are publicly available from the NCBI Sequence Read Archive (accession No. SRR8430192, SRR8430191, and SRR8430193). Bioinformatic analyses were performed as described previously (Shigematsu M, et al., 2020, RNA Biol, 17: 1060-1069; Shigematsu M, et al., 2019, PLoS Genet, 15: e1008469). Reads were mapped to 471 mature cyto tRNAs obtained from GtRNAdb (Chan P P, et al., 2009, Nucleic Acids Res, 37: D93-97), and then to mature rRNAs, to mRNAs of RefSeq with NM-staring accession numbers, to the mitochondrial genome (GenBank: CM001971.1 sequence plus 22 mitochondrial tRNA sequences), and to the whole genome (GRCh37/hg19).

In Vitro RNA Synthesis

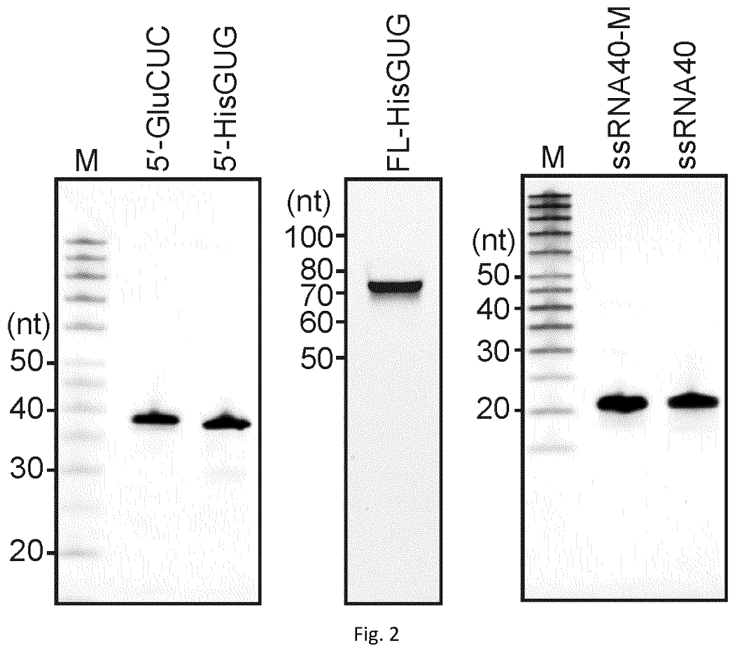

The synthetic RNAs used in this study are shown in Table 4 below. While antisense oligonucleotides, miRNAs, and a piRNA (spike-in) were synthesized by Integrated DNA Technologies, 5′-tRNA halves, FL-tRNA^{HisGUG}, and ssRNA40 were synthesized by an in vitro reaction as described previously (Shigematsu M, et al., 2017, RNA, 23: 161-168). dsDNA templates were synthesized using PrimeSTAR GXL DNA Polymerase (Takara Bio) and the primers shown in Table 5 below. The templates were then subjected to an in vitro transcription reaction with T7 RNA polymerase (New England Biolabs) at 37° C. for 4 h. For 5′-tRNA^{GluCUC} half production, the in vitro synthesized RNA contained the ribozyme sequence to generate a mature 5′-end as described previously (Fechter P, et al., 1998, FEBS Lett, 436: 99-103), so the reaction mixture was further incubated for three cycles at 90° C. for 2.5 min and 37° C. for 15 min, allowing the ribozyme reaction. The synthesized RNAs were then gel-purified using denaturing PAGE with single-nucleotide resolution, and the quality of the gel-purified RNAs was confirmed by denaturing PAGE as shown in FIG. 2. For FL-tRNA^{HisGUG} annealing was performed by incubating it in the annealing buffer consisting of 50 mM Tris-HCl (pH 8) and 100 mM MgCl₂ at 70° C. for 3 min, followed by incubation at 37° C. for 20 min. Low Molecular Weight Marker 10-100 nt (Affymetrix) was used as a marker in the denaturing PAGE.

TABLE 4
Sequences of synthetic RNAs/DNAs

| RNA | Sequence (5′-3′) |
|---|---|
| 5′-HisGUG | GCCGUGAUCGUAUAGUGGUUAGUACUCUGCGUUG (SEQ ID |
| | NO: 1) |
| 5′-HisGUG-Mod | GCCGUGAUCGUAUAGDGGDDAGUACUCUGCGψUG (SEQ ID |
| | NO: 2) |
| 5′-GluCUC | UCCCUGGUGGUCUAGUGGUUAGGAUUCGGCGCUC (SEQ ID |
| | NO: 3) |
| FL-HisGUG | GCCGUGAUCGUAUAGUGGUUAGUACUCUGCGUUGUGGCCGC |
| | AGCAACCUCGGUUCGAAUCCGAGUCACGGCA (SEQ ID NO: 4) |
| SSRNA40 | GCCCGUCUGUUGUGUGACUC (SEQ ID NO: 5) |
| ssRNA40-M | GCCCGACAGAAGAGAGACAC (SEQ ID NO: 6) |
| miR-21 | UAGCUUAUCAGACUGAUGUUGA (SEQ ID NO: 7) |
| miR-150 | UCUCCCAACCCUUGUACCAGUG (SEQ ID NO: 8) |
| piR-3 (spike-in) | UGAGAGUGGCAUCUAAAUGUUUAGUGGU (SEQ ID NO: 9) |
| AS-oligo | mG*mC*mC*mG*mT*mG*mA*mT*mC*mG*T*A*T*A*G*T*G*G*T |
| | *T*A*G*T*mA*mC*mT*mC*mT*mG*mC*mG*mT*mT*mG |
| Ctrl-oligo | mG*mT*mG*mT*mT*mC*mC*mG*mA*mT*G*T*G*G*C*T*C*T*G |
| | *G*A*C*T*mG*mG*mT*mA*mT*mC*mT*mA*mG*mT*mA |

“D” designates dihydrouridine; “ψ” designates pseudouridine; “mN” designates 2′-O-methylated nucleotide; and “*” designates phosphorothioate bond.

TABLE 5
Sequences of primers for the synthesis of dsDNA templates

| Target | Primer | Sequence (5′-3′) |
|---|---|---|
| 5′-tRNA^{HisGUG} half | Forward | GCTTAATACGACTCACTATAGCCGT (SEQ ID |
| | | NO: 10) |
| | Reverse | MCmAACGCAGAGTACTAACCACTATACGATCAC |
| | | GGCTACGATCACGGCTAGT (SEQ ID NO: 11) |
| 5′-tRNA^{GluCUC} half | Forward | CCTGCAGTAATACGACTCACTATAGGGAGAAGGG |
| | | ACTGATGAGTCCGTGAGGACGAAACGGTACCCGG |
| | | TACCGTCTCCCTGGTGGTCTAGTGGTTAG (SEQ ID |
| | | NO: 12) |
| | Reverse | mGmAGCGCCGAATCCTAACCACT (SEQ ID NO: 13) |
| SSRNA40 | Forward | GCTTAATACGACTCACTATAGCCCGT (SEQ ID |
| | | NO: 14) |
| | Reverse | mGmAGTCACACAACAGACGGGCTATAGT (SEQ ID |
| | | NO: 15) |
| SSRNA40-M | Forward | GCTTAATACGACTCACTATAGCCCGA (SEQ ID |
| | | NO: 16) |
| | Reverse | mGmUGTCTCTCTTCTGTCGGGCTATAGT (SEQ ID |
| | | NO: 17) |

“mN” designates 2′-O-methylated nucleotide.

Fluorescent Labeling of 5′-tRNA Halves and their EV-Mediated Delivery to Cells

The synthetic 5′-tRNA^{HisGUG} half and 5′-tRNA^{GluCUC} half were fluorescent-labeled at their 3′-end based on a previously-described method (Zearfoss N R, et al., 2012, Methods Mol Biol, 941: 181-193). In brief, synthetic RNAs were incubated in 100 mM NaOAc (pH 5.2) and 100 μM NaIO4 at room temperature for 90 min, followed by ethanol precipitation. Then the pellet was dissolved in a solution containing 1.5 mM FTSC (Cayman Chemical) and 100 mM NaOAc (pH 5.2), followed by overnight incubation at 4° C. After ethanol precipitation, the labeled RNAs were subjected to Centri-Spin 10 (Princeton Separations) purification to remove unreacted FTSC. Then, 80 pmol of the labeled RNA was transfected into HMDMs using RNAiMAX (Thermo Fisher Scientific). After 24 hours, the cells were washed with PBS and further incubated for 12 hours with LPS, and the cell culture medium was subjected to EV isolation as described above. The isolated EV fraction was then added to HMDMs, followed by incubation for 6 hours and visualization of the labeled 5′-tRNA halves with Rab7 and TLR7 by confocal microscopy as described below.

Immunofluorescence Staining and Confocal Microscopy

Immunofluorescence staining was performed as described previously (Honda S, et al., 2017, Sci Rep, 7: 4110) using anti-Rab7 (diluted 1:100, Cell Signaling Technology), anti-TLR7 (diluted 1:500, Novus Biologicals), and Alexa Fluor 488 goat anti-rabbit IgG (diluted 1:2000, Thermo Fisher Scientific) as primary and secondary antibodies, respectively. After DNA counterstaining with ProLong Gold Antifade Reagent with DAPI (Thermo Fisher Scientific), images were acquired using a Nikon Eclipse Ti-U confocal microscope at the Bioimaging Facility of the Sidney Kimmel Cancer Center at Thomas Jefferson University.

DOTAP-Mediate RNA Delivery to Endosomes

To deliver RNAs to endosomes, DOTAP liposomal transfection reagent (Sigma-Aldrich) was used as previously described (Fabbri M, et al., 2012, Proc Natl Acad Sci USA, 109: E2110-2116; Gantier M P, et al., 2008, J Immunol, 180: 2117-2124). In brief, 230 pmol or other various amounts of synthetic RNAs were mixed with 60 μl of HBS buffer and 15 μl of DOTAP reagent and incubated for 15 min. The RNA-DOTAP solution was then added to 1 ml HMDM or PHMDM medium, followed by incubation of the cells for 16 h.

EV-Mediate RNA Delivery to Endosomes

Synthetic 5′-tRNA^{HisGUG} half, 5′-tRNA^{GluCUC} half, and ssRNA40-M (80 pmol) were transfected to HMDMs (9×10⁶ cells) using RNAiMAX (Thermo Fisher Scientific). After 24 h, the cells were washed with PBS and further incubated for 12 h, and the cell culture medium was subjected to EV isolation as described above. The isolated EVs were then added to HMDMs (1×10⁶ cells), followed by incubation for 12 h, RNA extraction, and RT-qPCR quantification of TNFα, IL-1β, and IL-12p40 mRNAs.

Regarding experiments using antisense oligonucleotides, control oligonucleotides with scrambled sequences or antisense oligonucleotides for the 5′-tRNA^{HisGUG} half (Table 4 above) were first infused with DOTAP as described above. EVs isolated from LPS-treated HMDMs were mixed with the DOTAP-oligonucleotides solution and then were applied to recipient HMDMs, followed by incubation for 16 hours. To eliminate possible effects of potential endotoxin (LPS) contamination, EVs isolated from LPS-treated HMDMs were incubated with 10 mg/ml polymyxin B (PMB) (Sigma-Aldrich) at 4° C. for 1 hour prior to mixing with the DOTAP-oligonucleotides solution.

ELISA

For ELISA experiments, RNA transfection using DOTAP was performed in Opti-MEM (Thermo Fisher Scientific) and the culture medium from 1×10⁶ HMDMs or 1×10⁵ PHMDMs was subjected to ELISA (R&D Systems) for quantification of TNFα and IL-1β. Their absolute amounts were calculated based on standard curves.

RNAi KD of ANG, TLR7, and TLR8

To silence the expression of ANG, TLR7, and TLR8, siRNAs designed in previous reports (Honda S, et al., 2015, Proc Natl Acad Sci USA, 112: E3816-3825; Gantier M P, et al., 2008, J Immunol, 180: 2117-2124; Diebold S S, et al., 2004, Science, 303: 1529-1531) were synthesized by Bioland Scientific. Their sense strand sequences are 5′-AAACCUAAGAAUAAGCAAGUCAU-3′ (SEQ ID NO:52), 5′-GCCUUGAGGCCAACAACAUUU-3′ (SEQ ID NO:53), and 5′-GGUGGUGCUUCAAUUAAUAUU-3′ (SEQ ID NO:54) for ANG, TLR7, and TLR8, respectively. ON-TARGETplus Nontargeting siRNA #2 (D-001810-02, Dharmacon) was used as a negative control as previously described (Honda S, et al., 2015, Proc Natl Acad Sci USA, 112: E3816-3825). HMDMs were transfected with 50 nM of each siRNA using RNAiMAX (Thermo Fisher Scientific). In simultaneous knockdown of TL7 and TLR8, 100 nM of the siRNA mixture for TLR7/8 (50 nM each for TLR7 and TLR8) and 100 nM of control siRNA were used. In 60 hours after transfection, LPS were added and HMDMs were further incubated for 12 hours.

TLR7 KO THP-1 Cell Lines

TLR7 KO THP-1 cells were generated using the CRISPR/Cas9 system at Genome Editing Institute in ChristianaCare. Two different clones, KO #1 and KO #2, were generated using gRNA1 (5′-ACUUUCAGGUGUUUCCAAUG-3′; SEQ ID NO:55) and gRNA2 (5′-UAGGAAACCAUCUAGCCCCA-3′; SEQ ID NO:56), respectively. The KO cells were differentiated into HMDMs and used for transfection of DOTAP-fused RNAs as described above. Confirmation of TLR7 depletion in the KO cells was done using western blot analysis as described above.

Human Plasma Samples and RNA Isolation

Human plasma samples were derived from healthy or Mtb-infected males aged 30-35 years and obtained from BioIVT. For RNA isolation, 500 μl of plasma was first centrifuged at 16,060 g for 5 min, then 400 μl of supernatant was mixed with synthetic mouse piR-3 spike-in control (20 fmol) and subjected to RNA extraction using TRIzol LS (Thermo Fisher Scientific). The extracted RNAs were further subjected to purification using the miRNeasy Mini Kit (Qiagen). Based on the quantification of miR-451 and miR-23a-3p and calculation of “miR ratio” as described earlier (Shah J S, et al., 2016, PLoS One, 11: e0153200), no hemolysis was observed in any of the plasma samples. The extracted RNA samples were subjected to quantification of 5′-tRNA halves, and quantification of piR-3 (spike-in) was used for normalization.

The results of the present Example are now described herein.

BCG Infection and Surface TLR Activation Induce the Expression of 5′-tRNA Halves in HMDMs

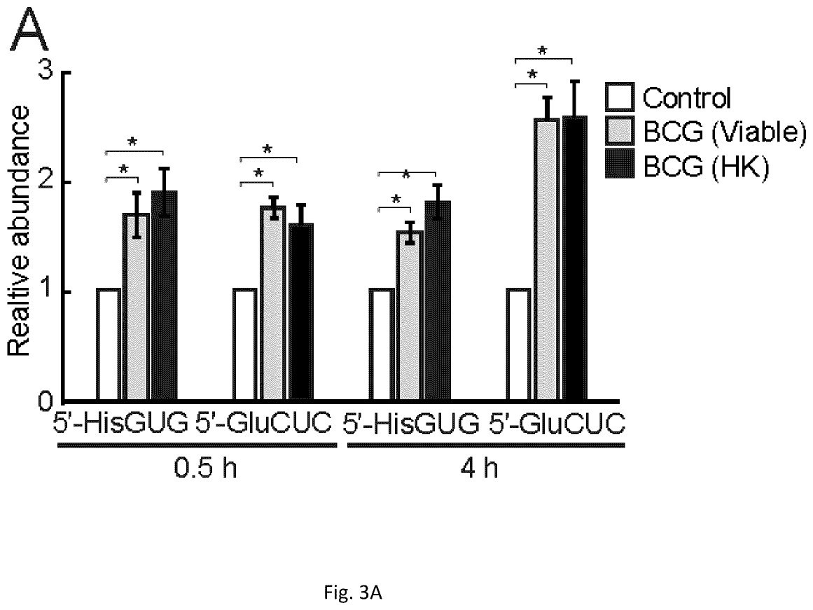

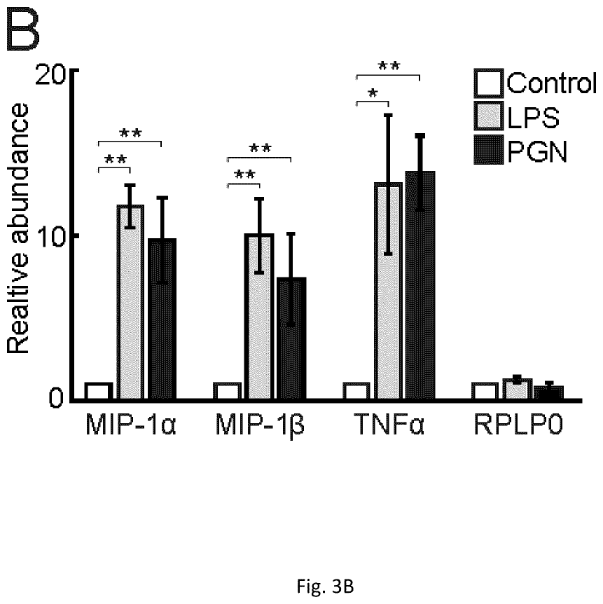

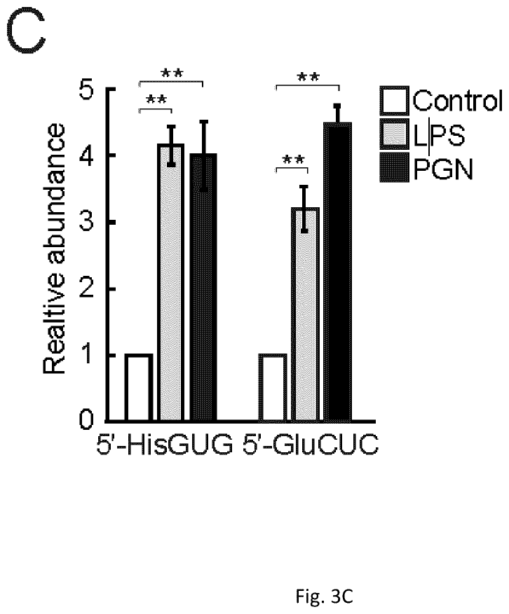

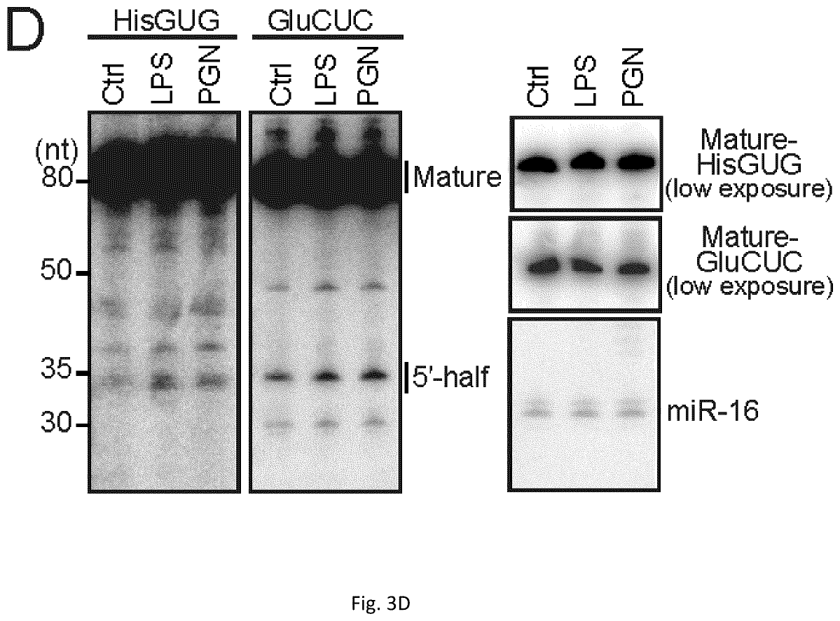

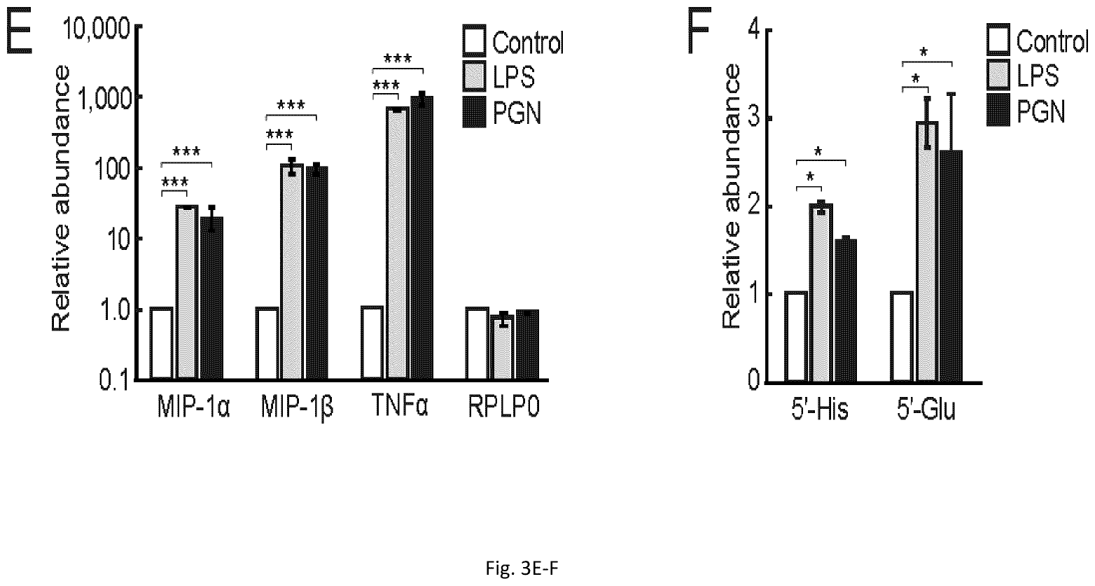

HMDMs express both surface and endosomal TLRs and have been used to study TLR pathways (Eng H L, et al., 2018, Biochem Biophys Res Commun, 497: 319-325; Nahid M A, et al., 2016, J Leukoc Biol, 100: 339-349), while BCG has been used as a model bacterium for tuberculosis infection (Minassian A M, et al., 2012, J Infect Dis, 205: 1035-1042). In the present Example, THP-1-derived HMDMs were infected with viable or heat-killed (HK) BCG, and two 5′-tRNA halves (5′-tRNA^{HisGUG} half and 5′-tRNA^{GluCUC} half, previously abundantly detected in human breast cancer cells [Honda S, et al., 2015, Proc Natl Acad Sci USA, 112: E3816-3825]) were quantified by tRNA half-specific TaqMan RT-qPCR (Honda S, et al., 2015, Proc Natl Acad Sci USA, 112: E3816-3825; Honda S, et al., 2017, Nucleic Acids Res, 45: 9108-9120), in which a 3′-adapter was ligated to the 5′-tRNA half and then the ligation products were quantified using a TaqMan probe targeting boundary of the adapter and the tRNA half. As shown in FIG. 3A, BCG infection enhanced the expression of both of the 5′-tRNA halves. The induction of 5′-tRNA half expression was independent of the viability of BCG (FIG. 3A), suggesting that the induction could result from the pathway of surface TLRs, which recognize BCG PAMPs, or from the process of endocytosis. To examine the involvement of surface TLRs in 5′-tRNA half expression, TLR4 and TLR2 were stimulated by treating HMDMs with lipopolysaccharide (LPS) or peptidoglycan (PGN), respectively (Guan R, et al., 2007, Trends Microbiol, 15: 127-134; Medzhitov R, 2001, Nat Rev Immunol, 1: 135-145). Successful stimulations of the TLRs were confirmed by upregulation of tumor necrosis factor α (TNFα) and the macrophage inflammatory factors, MIP-1α and MIP-1β (FIG. 3B). Upon stimulation of the surface TLRs, the expression of 5′-tRNA halves was observed to be upregulated by TaqMan RT-qPCR (FIG. 3C) and northern blot (FIG. 3D). Notably, the expression levels of corresponding mature tRNAs were unchanged by surface TLR stimulation (FIG. 3D). As described in previous studies (Yamasaki S, et al., 2009, J Cell Biol, 185: 35-42; Honda S, et al., 2015, Proc Natl Acad Sci USA, 112: E3816-3825; Honda S, et al., 2017, Nucleic Acids Res, 45: 9108-9120; Thompson D M, et al., 2009, Cell, 138: 215-219), the production of 5′-tRNA halves did not influence the levels of mature tRNAs which are steadily maintained by an unknown mechanism. Primary human monocyte-derived macrophages (PHMDMs) differentiated from CD14+ monocytes were further analyzed. As in the case of HMDMs, treatment of PHMDMs with LPS or PGN caused surface TLR activation (FIG. 3E) and upregulation of 5′-tRNA half expression (FIG. 3F), confirming the surface TLR-induced expression of 5′-tRNA halves in the primary cells of the human body.

Surface TLR-Activated NF-κB Upregulates the Expression of ANG mRNA

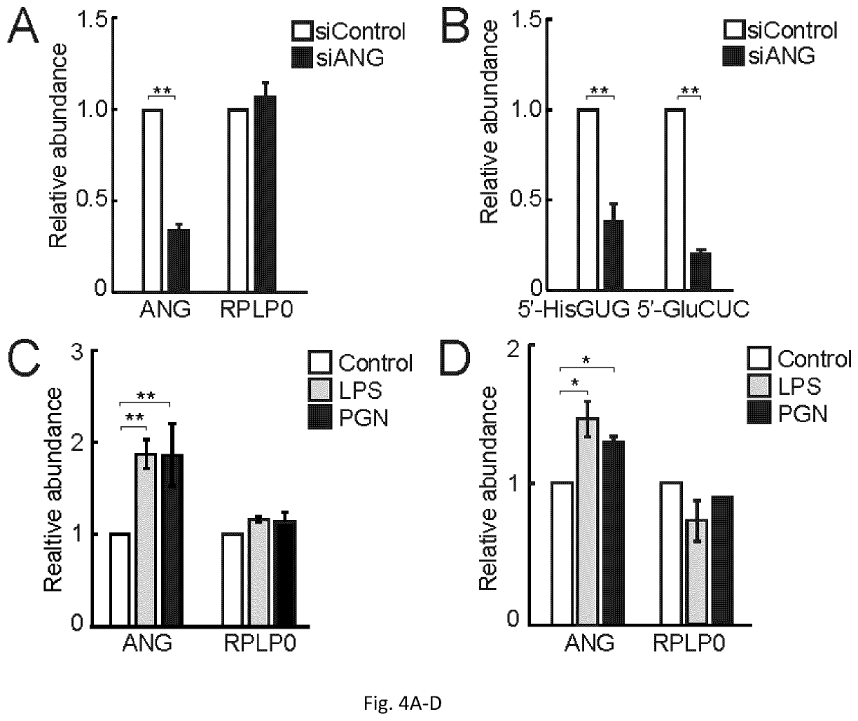

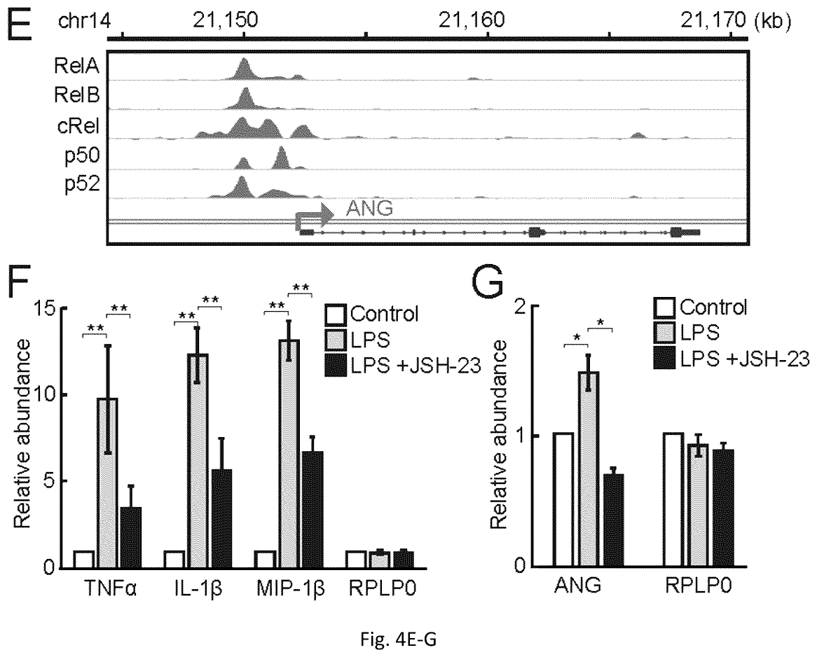

In mammalian cells, ANG cleaves the anticodon-loops of tRNAs to produce tRNA halves (Fu H, et al., 2009, FEBS Lett, 583: 437-442; Yamasaki S, et al., 2009, J Cell Biol, 185: 35-42; Honda S, et al., 2015, Proc Natl Acad Sci USA, 112: E3816-3825). To confirm the involvement of ANG in the tRNA half production in LPS-treated HMDMs, siRNA-mediated knockdown (KD) of ANG expression was performed, which reduced the ANG mRNA levels to around 35% (FIG. 4A). The ANG KD decreased the expression of 5′-tRNA halves (FIG. 4B), suggesting that tRNA halves are generated by ANG-mediated cleavage of tRNAs in LPS-treated HMDMs. Because the expression levels of ANG mRNA were upregulated upon LPS or PGN treatment in HMDMs (FIG. 4C) and PHMDMs (FIG. 4D), it was reasoned that the transcription factors downstream of surface TLR signal transduction pathways, such as NF-fB, could induce the expression of ANG mRNA. Indeed, direct binding of NF-fB to the region upstream of the ANG gene was suggested by chromatin immunoprecipitation and sequencing (ChIP-seq) data for NF-fB in lymphoblastoid B cells (FIG. 4E; Zhao B, et al., 2014, Cell Rep, 8: 1595-1606). The potential involvement of NF-κB in ANG mRNA expression was examined by treating HMDMs with JSH-23, an inhibitor of NF-κB (Chen D, et al., 2018, Nat Commun, 9: 873; Li J Y, et al., 2014, Cell Mol Immunol, 11: 477-494), which reduced the immune response as expected (FIG. 4F). ANG mRNA levels were unchanged when HMDMs were treated with NF-κB inhibitor and LPS (FIG. 4G), suggesting that NF-κB-mediated transcription upregulates ANG mRNA, which would increase the levels of ANG protein, possibly leading to enhanced tRNA cleavage for induction of tRNA half expression by surface TLR activation.

5′-tRNA Halves are Massively Accumulated in EVs Secreted from HMDMs

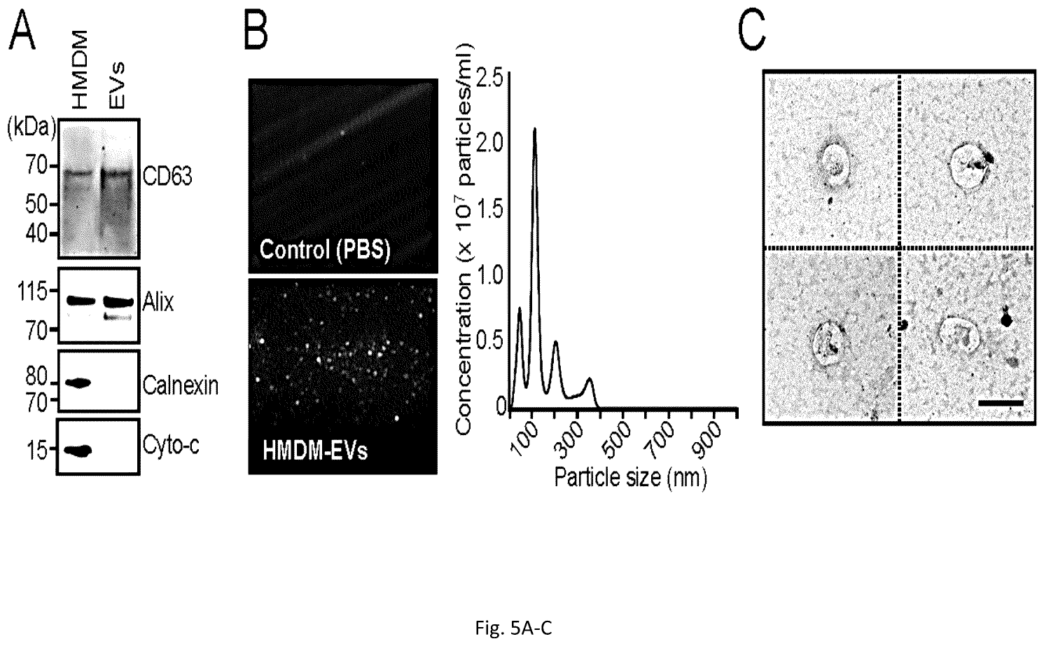

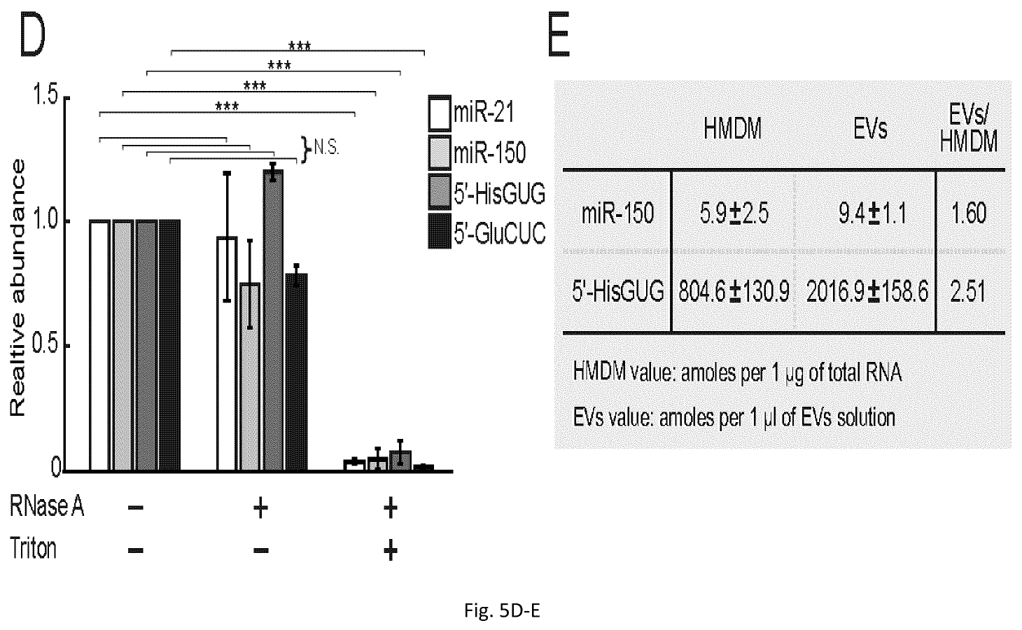

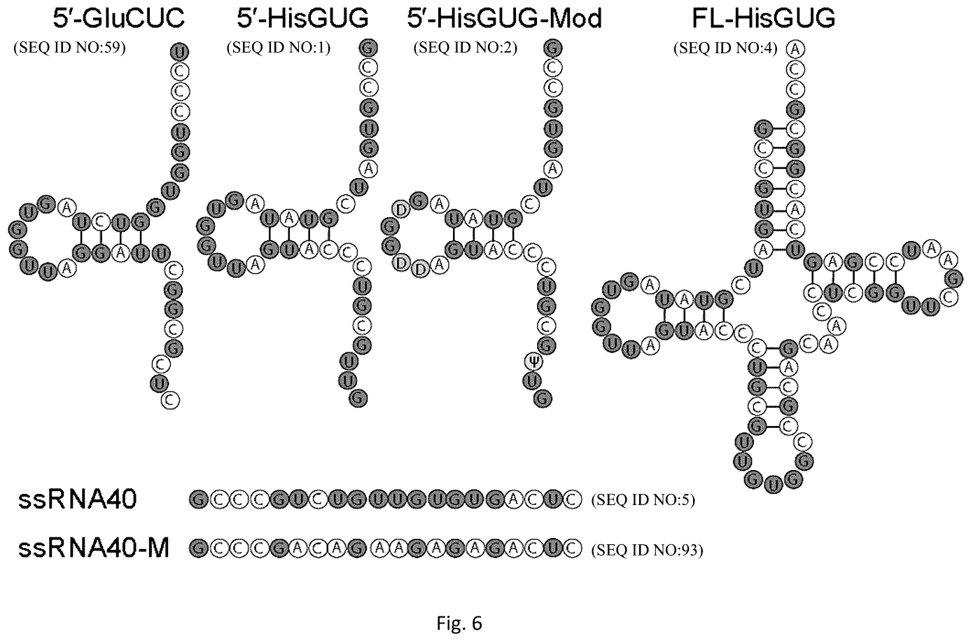

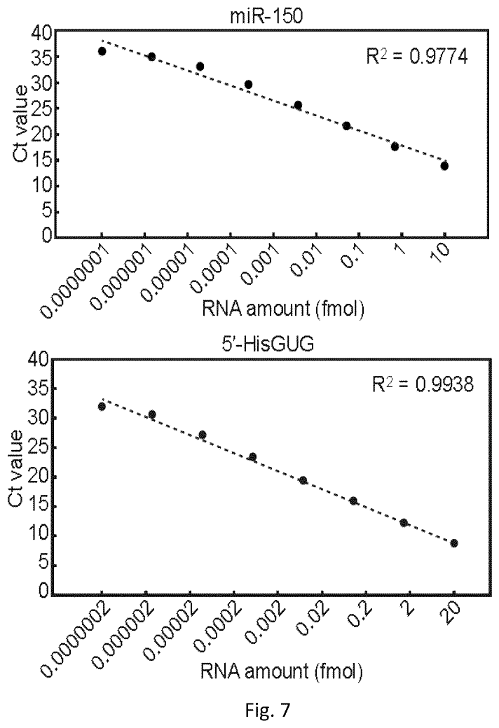

To explore whether EVs secreted from HMDMs are carriers of tRNA halves, the EVs from the culture medium of LPS-treated HMDMs were isolated by an ultracentrifugation-based method. Western blots for the isolated EVs confirmed the presence of CD63 and Alix, proteins known for EV accumulation (Zhang Y, et al., 2010, Mol Cell, 39: 133-144; Holder B, et al., 2016, Traffic, 17: 168-178), and the absence of calnexin and cytochrome c, which are non-EV proteins (FIG. 5A; Lobb R J, et al., 2015, J Extracell Vesicles, 4: 27031; Tang Y T, et al., 2017, Int J Mol Med, 40: 834-844). Nanoparticle tracking analysis (NTA) showed the abundant presence of EVs from 80 to 120 nm at a high concentration (˜2.0×10⁷ particles/ml EV solution) (FIG. 5B). The isolated EVs were further observed by transmission electron microscopy (FIG. 5C), the results of which collectively confirmed the successful isolation of HMDM EVs. The isolated EVs were subjected to TaqMan RT-qPCR for two 5′-tRNA halves, 5′-tRNA^{HisGUG} half and 5′-tRNA^{GluCUC} half, as well as to stem-loop RT-qPCR for two miRNAs, miR-21 and miR-150, which are known to abundantly accumulate in HMDM EVs (Zhang Y, et al., 2010, Mol Cell, 39: 133-144). Clear amplification signals were obtained from all of the four examined RNAs. While the EVs treated with RNase alone yielded similar amplification signals to untreated EVs, the EVs treated with both RNase and detergent yielded drastically reduced amplification signals (FIG. 5D), confirming that the detected 5′-tRNA halves and miRNAs were present inside the isolated EVs and were not captured as non-EV contaminants. The absolute amounts of the 5′-tRNA^{HisGUG} half and miR-150 in LPS-treated HMDMs and their EVs were further explored. The calculation of the amounts was based on the standard curve from synthetic RNAs, which showed excellent linearity between input amounts and amplification signals (FIG. 2, FIG. 6 and FIG. 7). The determined abundances of the two RNAs per ∝g of total HMDM RNA or per ∝l of EV fraction are shown in FIG. 5E. Although miR-150 was reported as the most abundant miRNA species expressed in HMDMs and their EVs (Zhang Y, et al., 2010, Mol Cell, 39: 133-144), the abundance of the 5′-tRNA^{HisGUG} half was much more pronounced than that of miR-150; 136-fold and 215-fold higher in HMDMs and EVs, respectively.

5′-tRNA Halves are Produced from Specific tRNA Species in HMDMs and are Selectively Packaged into EVs

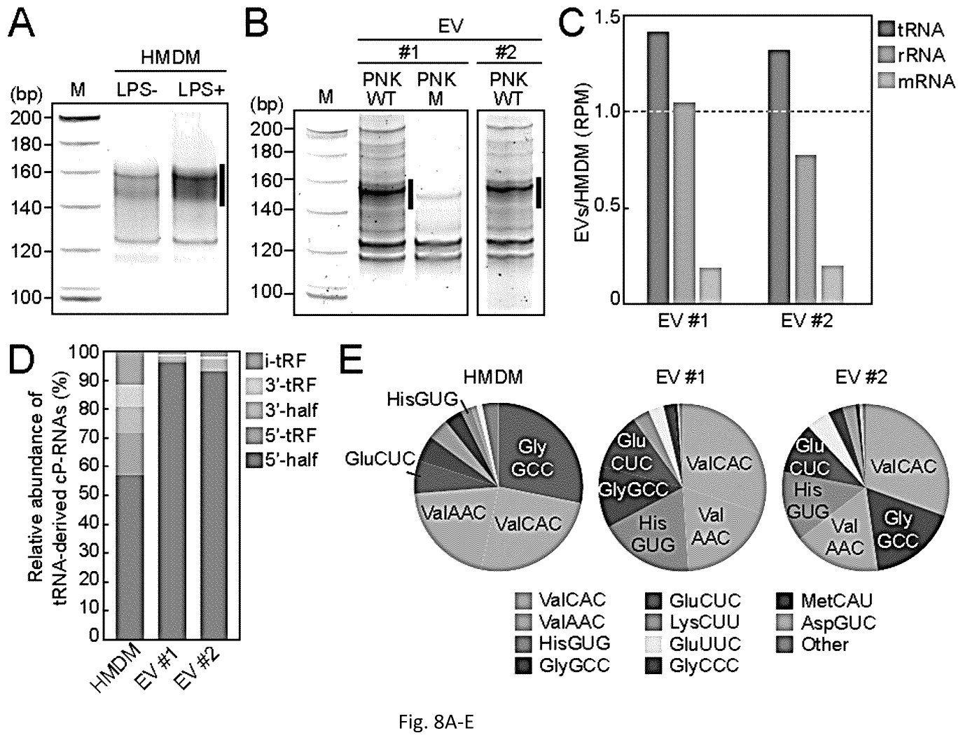

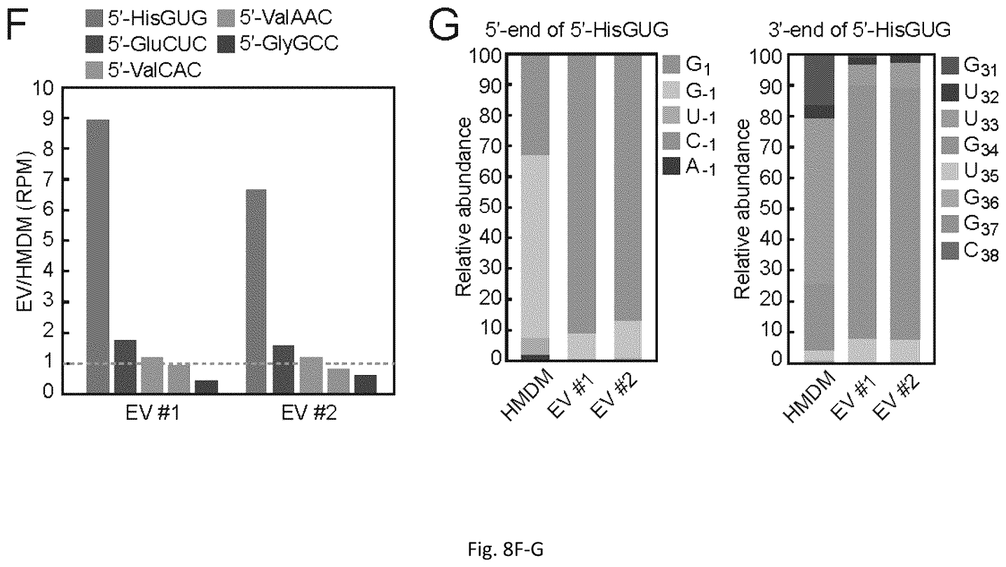

Given the abundant accumulation of 5′-tRNA halves in HMDMs and their EVs, the expression profiles of the 5′-tRNA halves were next identified. Although short RNA-seq was previously performed for HMDMs and their EVs (McDonald M K, et al., 2014, Pain, 155: 1527-1539; Cai C, et al., 2018, Front Immunol, 9: 723), standard RNA-seq cannot accurately capture 5′-tRNA halves because they possess a cP at their 3′-end that hinders adapter ligation (Honda S, et al., 2015, Proc Natl Acad Sci USA, 112: E3816-3825). Instead, “cP-RNA-seq” was employed, which can selectively amplify and sequence cP-RNAs, namely 5′-tRNA halves (Honda S, et al., 2015, Proc Natl Acad Sci USA, 112: E3816-3825; Honda S, et al., 2016, Nat Protoc, 11: 476-489). The cP-RNA-seq procedure was first applied to gel-purified short RNA fractions of HMDMs, which successfully amplified ˜140-160-bp bands (considering adapters' lengths, inserted RNA sequences were estimated to be ˜22-42-nucleotides [nt] in length) (FIG. 8A). Consistent with the upregulation of HMDM tRNA half expression by LPS treatment (FIG. 3), cP-RNA-seq amplified more abundant cDNAs from the LPS-treated HMDMs than from the untreated cells (FIG. 8A). In contrast, attempts to amplify clear cDNA bands from the RNAs of HMDM EVs by cP-RNA-seq were unsuccessful, possibly due to the limited amounts of EV-RNAs present. The cP-RNA-seq procedure includes a periodate oxidation step, which might be harsh enough to damage whole RNAs if the initial RNA amounts are limited. Therefore, for EV-RNAs, the decision was made to capture all short RNA species containing not only a cP but also a phosphate (P) or a hydroxyl group (OH) at the 3′-end. For this, EV-RNAs were first treated with T4 polynucleotide kinase (T4 PNK), which can remove cP and P from the 3′-end of RNAs, and then were subjected to the short RNA-seq procedure. This yielded abundant ˜140-160-bp cDNA bands (FIG. 8B), similar to the bands obtained from cP-RNA-seq of HMDMs (FIG. 8A). Interestingly, RNAs treated with a mutant T4 PNK, which lacks 3′-dephosphorylation activity (Wang L K, et al., 2002, Nucleic Acids Res, 30: 1073-1080), yielded only faint cDNA bands, suggesting that the majority of short RNA species in EVs contain a 3′-terminal cP or P and RNAs containing a 3′-OH end, such as miRNAs, are the minor species in EVs; this is consistent with the experimental results shown in FIG. 5E.

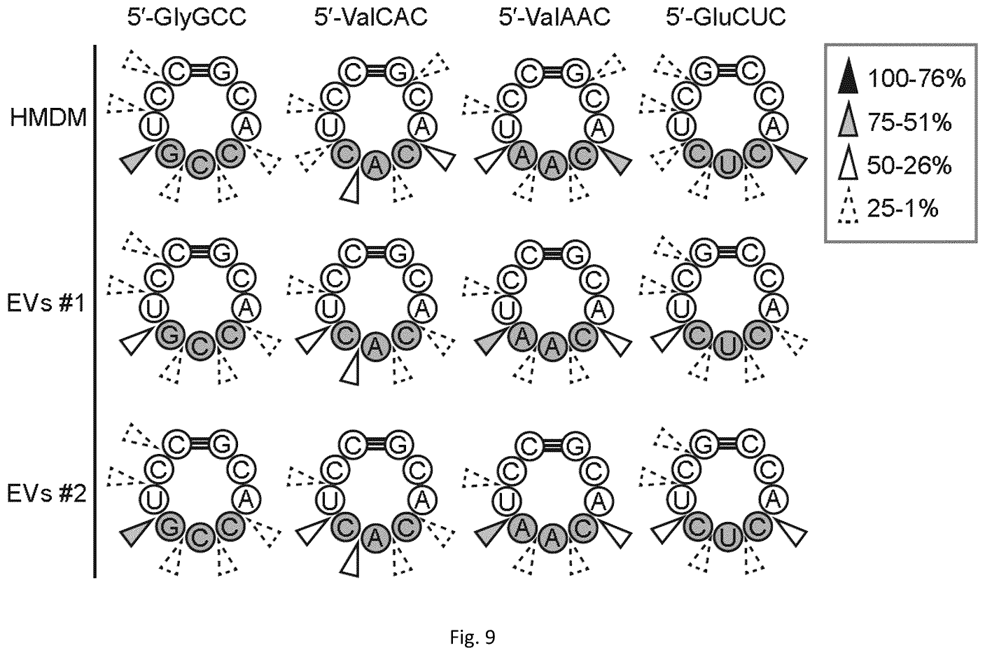

Illumina sequencing of the gel-purified ˜140-160-bp cDNAs from HMDMs and their EVs yielded approximately 35-44 million raw reads, of which >82-95% were extracted as reads with a length of 25-50 nt (FIG. 1). tRNA-mapped reads were enriched in EV libraries (FIG. 8C); among them, the 5′-tRNA halves were the most major species, as expected (FIG. 8D). While 5′-tRNA halves comprised ˜57% of tRNA-derived reads in HMDMs, they accounted for over 93% of tRNA-derived reads in EVs, suggesting that 5′-tRNA halves could be selectively packaged into EVs to a greater extent than other tRNA-derived RNAs. Considering that the human genome encodes 55 cytoplasmic (cyto) tRNA isoacceptors with different anticodon sequences (Chan P P, et al. 2009, Nucleic Acids Res, 37: D93-97), the identified 5′-tRNA halves were derived from a rather focused subset of tRNAs, such as cyto tRNA^{ValCAC}, tRNA^{ValAAC} tRNA^{GlyGCC}, tRNA^{HisGUG}, and tRNA^{GluCUC}, which are in aggregates the sources of 88-90% of the identified 5′-tRNA halves in EVs (FIG. 8E). Among the five major 5′-tRNA halves, the relative abundance of the 5′-tRNA^{HisGUG} half in EVs was considerably greater than that in HMDMs, while the other four 5′-tRNA halves were similarly abundant in both libraries (FIGS. 8E and 8F), implying preferential incorporation of the 5′-tRNA^{HisGUG} half into EVs. tRNA^{HisGUG} contains an additional nucleotide at nucleotide position (np; according to the nucleotide numbering system of tRNAs [Sprinzl M, et al., 1998, Nucleic Acids Res, 26: 148-153]) −1 of its 5′-end. A recent analyses of BT-474 human breast cancer cells showed that the majority (˜60%) of cyto tRNA^{HisGUG} contains G₋₁, but a significant proportion contains U₋₁ or lacks the −1 nucleotide (contains G₁ as a 5′-terminal nucleotide; Shigematsu M, et al., 2017, RNA, 23: 161-168). As shown in FIG. 8G, while the 5′-tRNA^{HisGUG} half containing G₋₁ was the major species in HMDMs, the majority of the 5′-tRNA^{HisGUG} halves in EVs lacked the −1 nucleotide (G₁). Similarly, while the major 3′-terminal nucleotide was U33 for HMDM 5′-tRNA^{HisGUG} halves, the majority of the EV-5′-tRNA^{HisGUG} halves contained G₃₄ as the 3′-end. The 5′-tRNA^{HisGUG} half from G₁ to G₃₄ comprised approximately 80% of EV-5′-tRNA^{HisGUG} halves but only 5% of HMDM 5′-tRNA^{HisGUG} halves. Inconsistency of the identified species between HMDMs and EVs was also observed in some other major 5′-tRNA half species (FIG. 9), implying that the efficiency of EV-loading may not be equal for all 5′-tRNA halves and specific species could be preferentially packaged into EVs.

EV-5′-tRNA Halves are Delivered into Endosomes in Recipient HMDMs

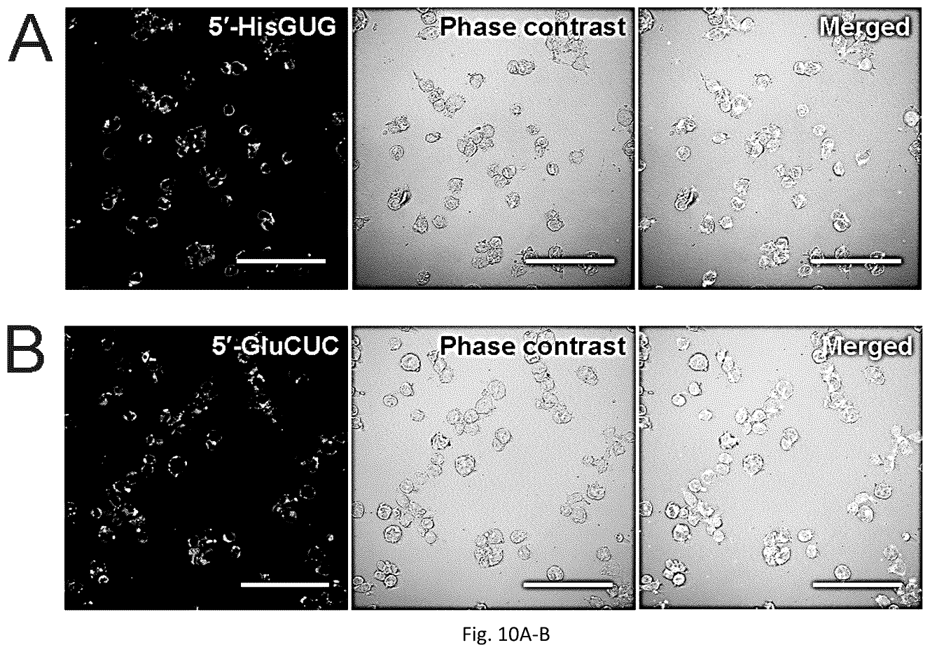

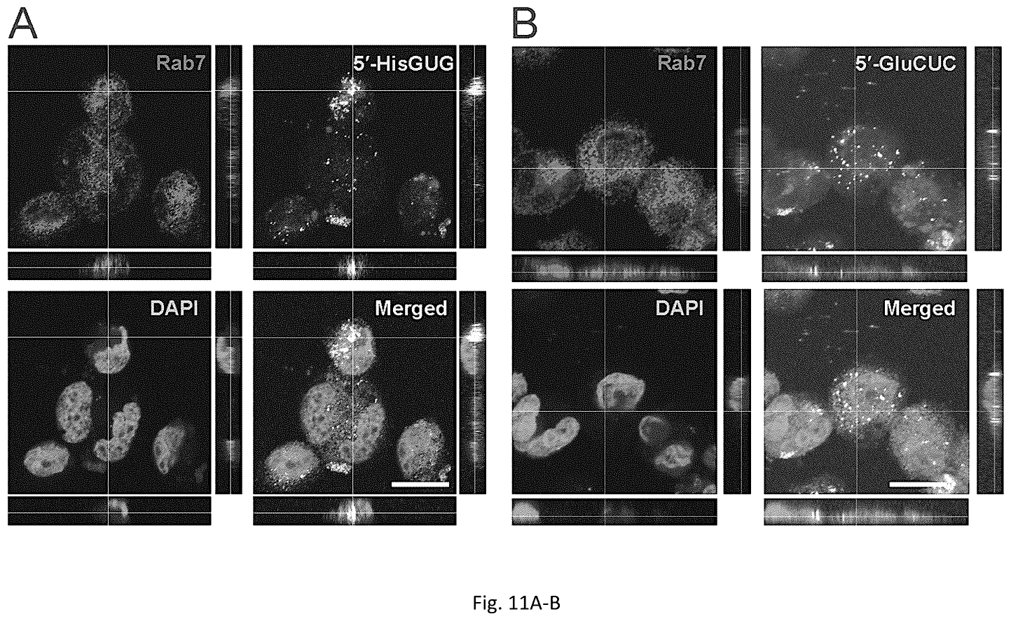

Because EV-miRNAs have been shown to be ligands for endosomal TLRs (Lehmann S M, et al., 2012, Nat Neurosci, 15: 827-835; Fabbri M, et al., 2012, Proc Natl Acad Sci USA, 109: E2110-2116), whether the abundantly-identified EV-tRNA halves are delivered into endosomes in recipient cells was examined. Synthetic 5′-tRNA^{HisGUG} half or 5′-tRNA^{GluCUC} half was chemically tagged with fluorescein-5-thiosemicarbazide (FTSC) (Zearfoss N R, et al., 2012, Methods Mol Biol, 941: 181-193) and transfected it into HMDMs, as shown in FIG. 10A-B. The EVs containing the labeled 5′-tRNA halves from the transfected cells were then isolated and subsequently applied to recipient HMDMs. As a result, the incorporation of the labeled EV-5′-tRNA halves into recipient cells was observed. Clear overlap between the signals of the 5′-tRNA halves and Rab7 (FIG. 11A-B), an endosome marker (Shearer L J, et al., 2019, Heliyon, 5: e02375), and TLR7 (FIG. 12A-B) confirmed the delivery of EV-tRNA halves into the endosomes of the recipient HMDMs. These results experimentally proved that tRNA halves in HMDMs are packaged into EVs and secreted outside of the cells, which are then delivered into the endosomes of recipient cells.

5′-tRNA^{HisGUG} Half Activates Endosomal TLR7

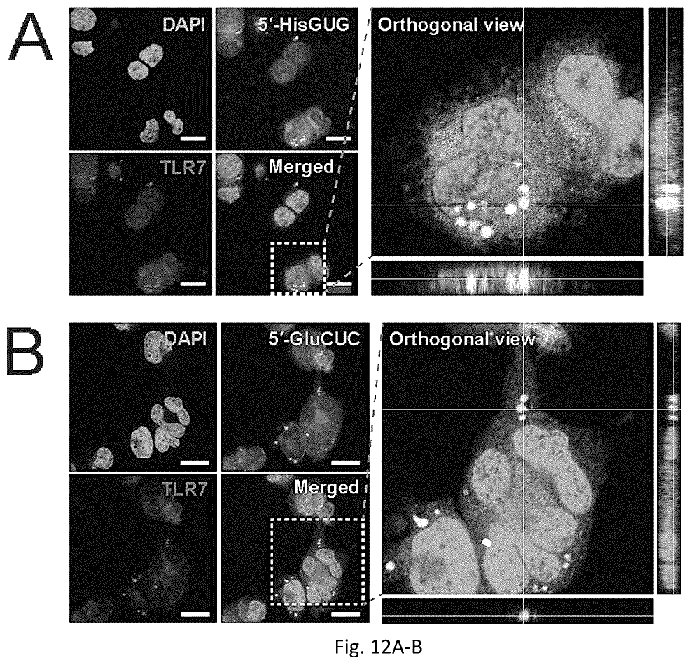

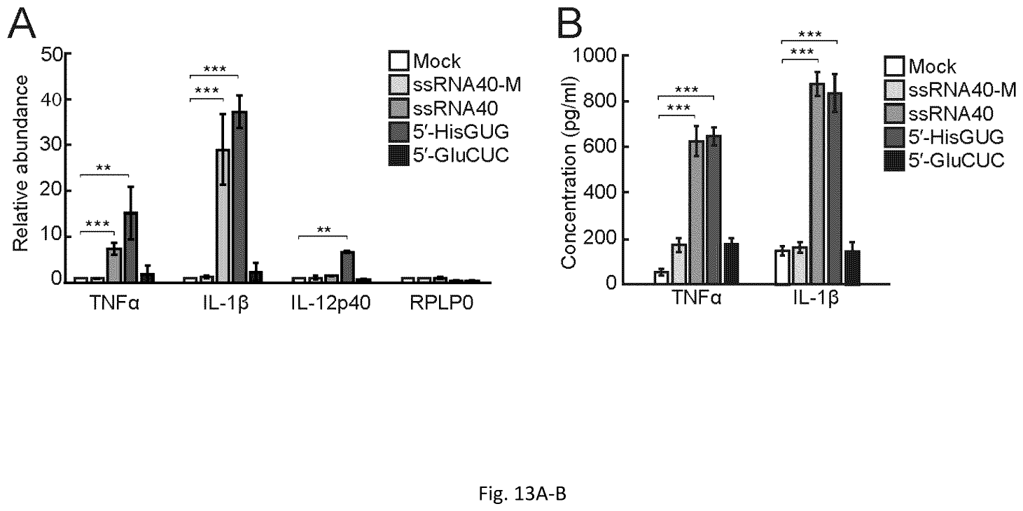

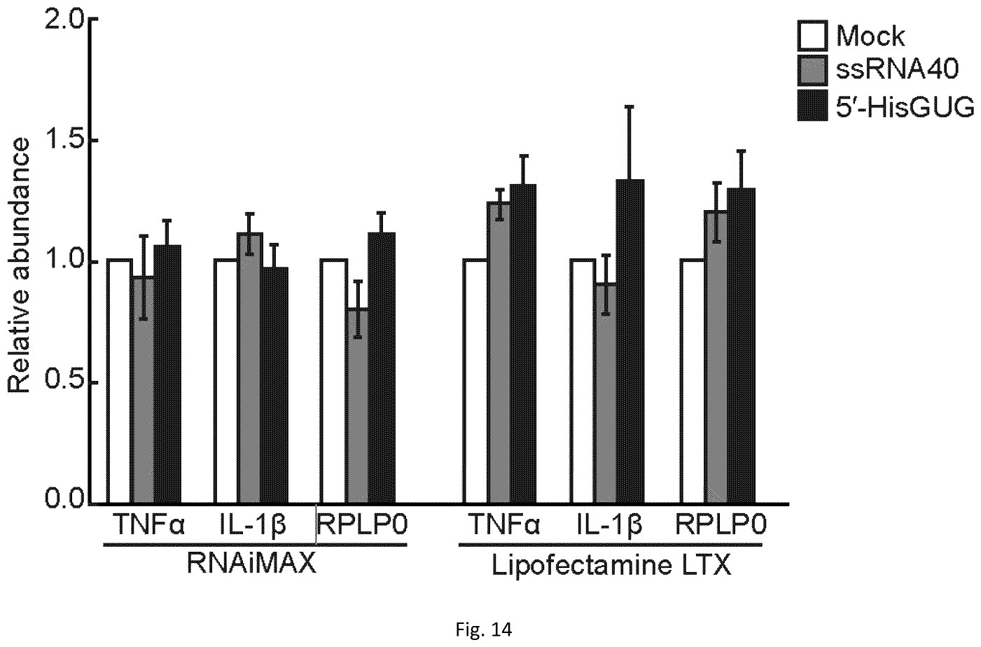

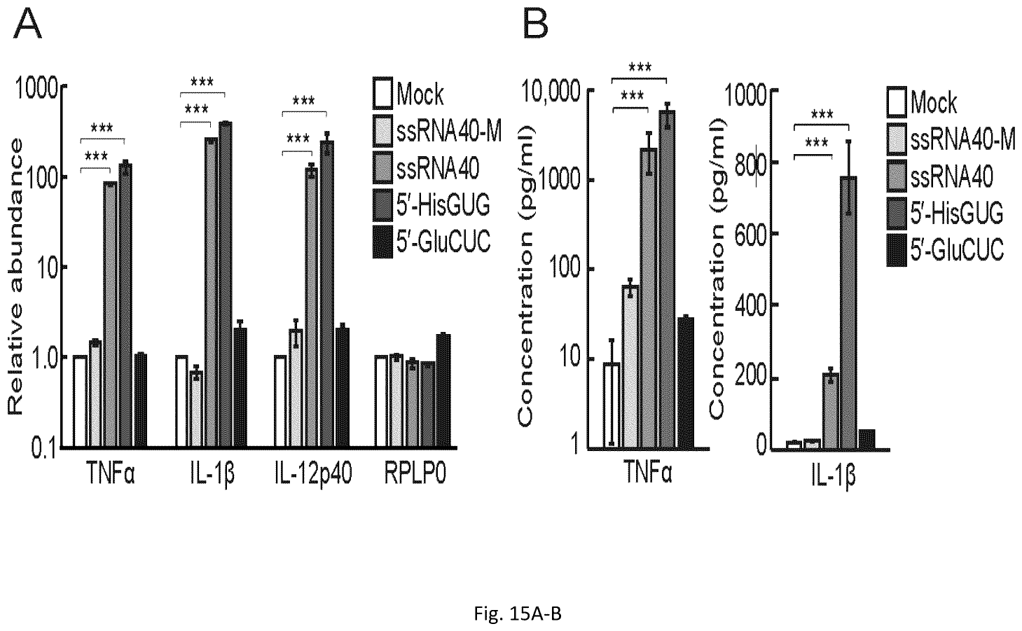

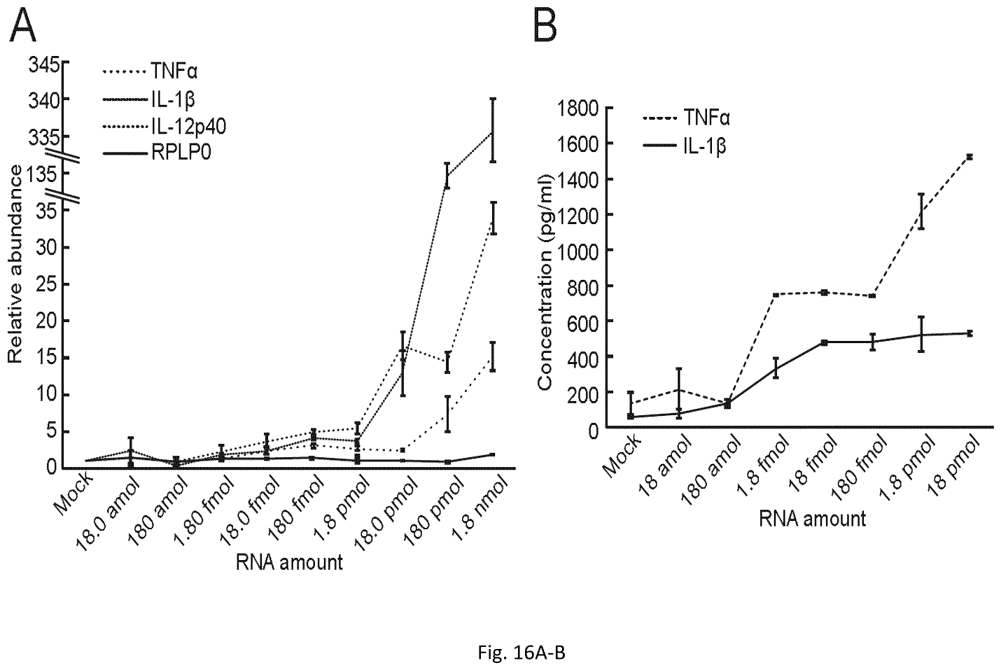

Given the abundant accumulation and endosome-targeted delivery of 5′-tRNA halves in HMDM EVs, the activity of the 5′-tRNA halves in stimulating ssRNA-sensing endosomal TLRs (i.e., TLR7 and -8) was further assessed. As described in earlier studies (Fabbri M, et al., 2012, Proc Natl Acad Sci USA, 109: E2110-2116; Gantier M P, et al., 2008, J Immunol, 180: 2117-2124), HMDMs were primed with interferon γ and then transfected with 5′-tRNA^{HisGUG} half or 5′-tRNA^{GluCUC} half using the cationic liposome 1,2-dioleoyloxy-3-trimethylammonium-propane (DOTAP) which mimics EVs. As controls, a 20-nt HIV-1-derived ssRNA termed ssRNA40 (FIG. 2 and FIG. 6), known to strongly activate endosomal TLRs (Heil F, et al., 2004, Science. 303: 1526-1529), and its inactive mutant (ssRNA40-M), in which U is replaced with A, were also transfected. As shown in FIG. 13A, transfections of the 5′-tRNA^{HisGUG} half and ssRNA40, a positive control, increased the production of TNFα, IL-1β, and IL-12p40 mRNAs, whereas transfections of the 5′-tRNA^{GluCUC} half and ssRNA40-M, a negative control, did not. Induction of the secretion of TNFα and IL-1β into culture medium upon the transfection of the 5′-tRNA^{HisGUG} half, as well as ssRNA40, was further confirmed by ELISA (FIG. 13B). Transfection of the 5′-tRNA^{HisGUG} half using Lipofectamine reagents did not show such inductions (FIG. 14), confirming that the delivery of 5′-tRNA^{HisGUG} half to endosomes, not to the cytoplasm, is necessary for the inductions. The strong activation of endosomal TLR by the DOTAP-fused 5′-tRNA^{HisGUG} half was further observed in PHMDMs. Upon transfection of 5′-tRNA^{HisGUG} half into PHMDMs, increased production of TNFα, IL-1β, and IL-12p40 mRNAs (FIG. 15A) and enhanced secretion of TNFα, and IL-1β (FIG. 15B) were observed. While the calculation of FIG. 5E indicated the presence of 25 fmol of EV-5′-tRNA^{HisGUG} half per 1 ml of medium (1 μl of EV solution was obtained from 80 μl of medium), 1.8 fmol of 5′-tRNA^{HisGUG} half per 1 ml of medium was sufficient to observe the activation of endosomal TLRs in PHMDMs (FIG. 16A-B), suggesting that physiologically-relevant amounts of 5′-tRNA^{HisGUG} half can activate endosomal TLR. Earlier studies have shown that modified nucleotides in tRNAs can affect endosomal TLR activation (Jockel S, et al., 2012, J Exp Med, 209: 235-241; Gehrig S, et al., 2012, J Exp Med, 209: 225-233). In the region of the 5′-tRNA^{HisGUG} half, mature tRNA^{HisGUG} contains the following five post-transcriptionally modified nucleotides: dihydrouridine (D) at np 16, 19, and 20; peudouridine (Ψ) at np 32, and queuosine (Q) at np 34 (Rosa M D, et al., 1983, Nucleic Acids Res, 11: 853-870; Clark W C, et al., 2016, RNA, 22: 1771-1784; Fergus C, et al., 2015, Nutrients, 7: 2897-2929). Among the five modified nucleotides, Q34 has been reported to block ANG-mediated anticodon-cleavage (Wang X, et al., 2018, RNA, 24: 1305-1313) and thus would be absent in the 5′-tRNA^{HisGUG} half. The synthetic 5′-tRNA^{HisGUG} half containing the other four modified nucleotides (FIG. 6) activated endosomal TLRs as strongly as unmodified RNA (FIG. 17A), suggesting that the endogenous, modified 5′-tRNA^{HisGUG} half would have the activity. Although mature tRNAs have been reported to be incorporated in EVs (Shurtleff M J, et al., 2017, Proc Natl Acad Sci USA, 114: E8987-E8995; Nolte-'t Hoen E N, et al., 2012, Nucleic Acids Res, 40: 9272-9285), interestingly, the full-length tRNA^{HisGUG} was incapable of stimulating endosomal TLR (FIG. 17B) possibly due to its rigid secondary and tertiary structures. These results suggest that shortening mature tRNA^{HisGUG} into less-rigid 5′-half molecules by anticodon-cleavage is necessary to activate endosomal TLR.

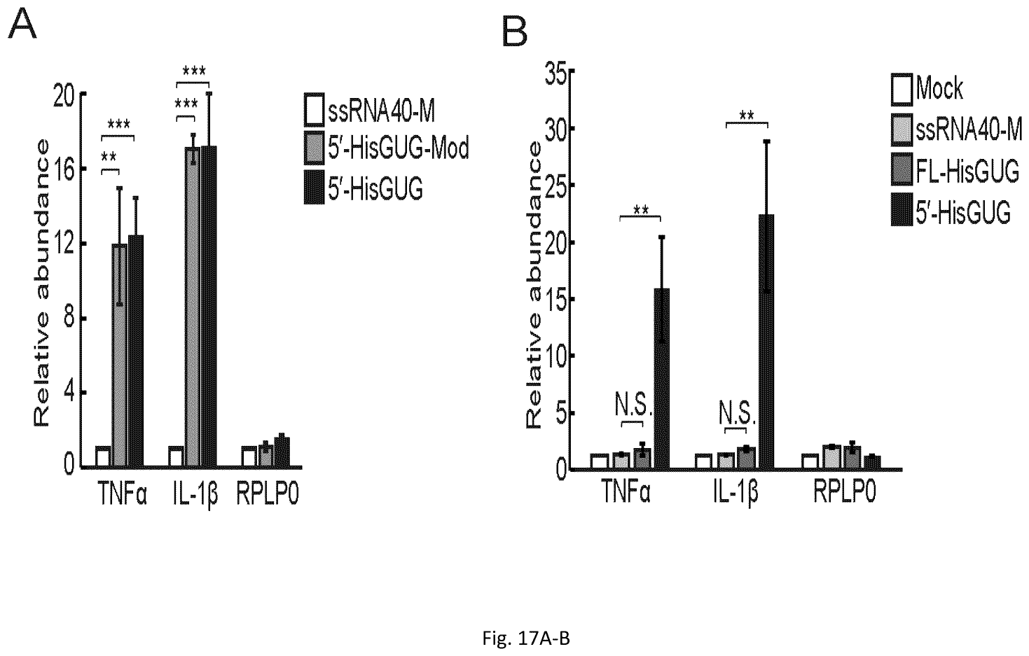

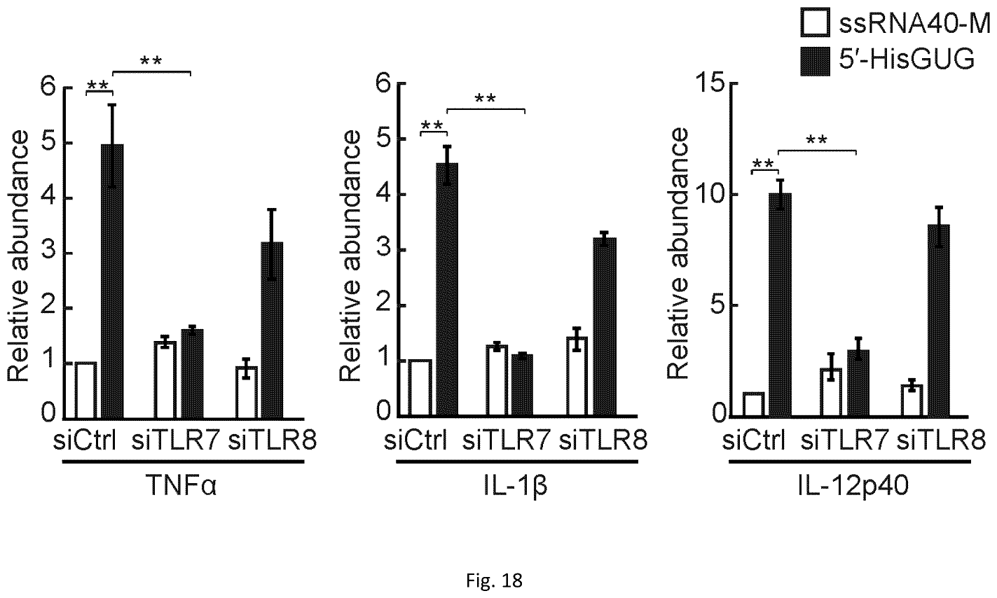

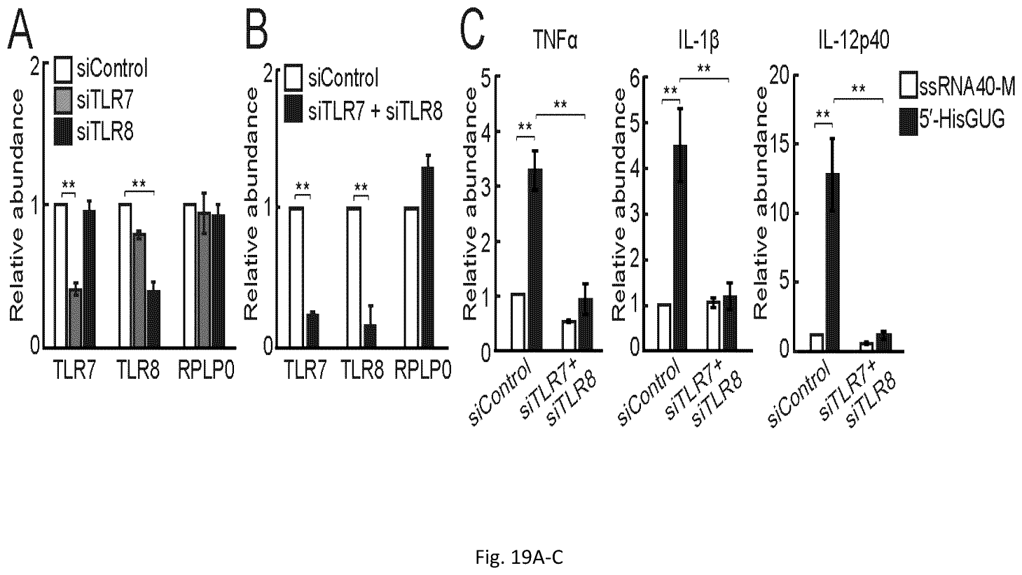

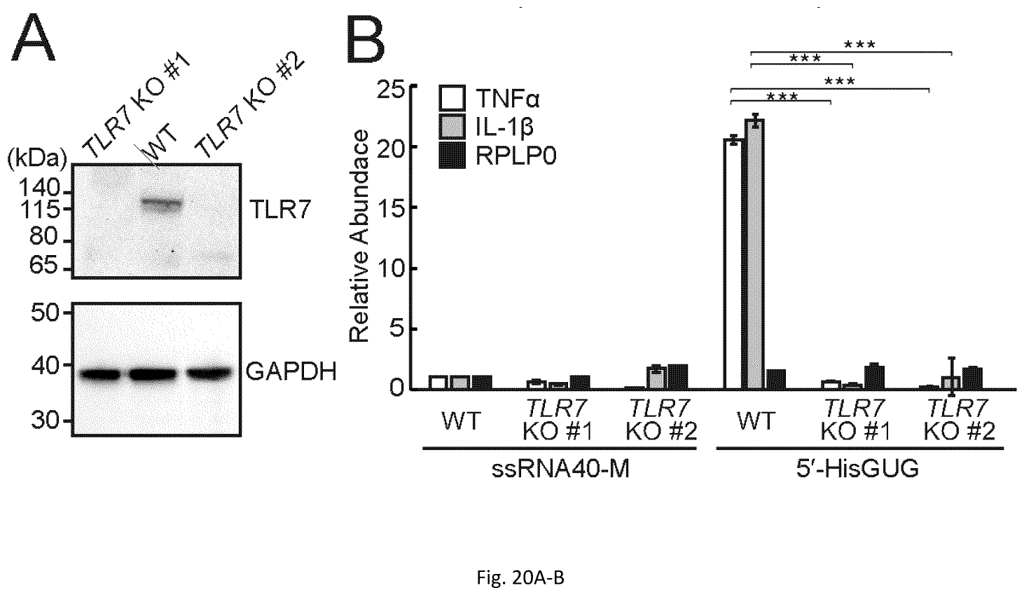

Next, whether the 5′-tRNA^{HisGUG} half activates endosomal TLR7 and/or TLR8 was examined. siRNA-mediated KD of TLR7 alone or simultaneous knockdown of TLR7 and -8 in HMDMs abolished the upregulation of TNFα, IL-1β, and IL-12p40 by DOTAP transfection of the 5′-tRNA^{HisGUG} half, whereas TLR8 KD alone did not (FIG. 18 and FIG. 19A-C). These results suggest that 5′-tRNA^{HisGUG} half stimulates endosomal TLR7 as strongly as ssRNA40, but not TLR8. To further confirm the involvement of TLR7 in the activity of the 5′-tRNA^{HisGUG} half, by using CRISPR/Cas9 approach, TLR7 knockout (KO) THP-1 cell lines were generated in which TLR7 expression is completely abolished (FIG. 20A). The 5′-tRNA^{HisGUG} half did not show the activity to stimulate endosomal TLR in TLR7 KO cells (FIG. 20B), confirming that the 5′-tRNA^{HisGUG} half activates endosomal TLR7.

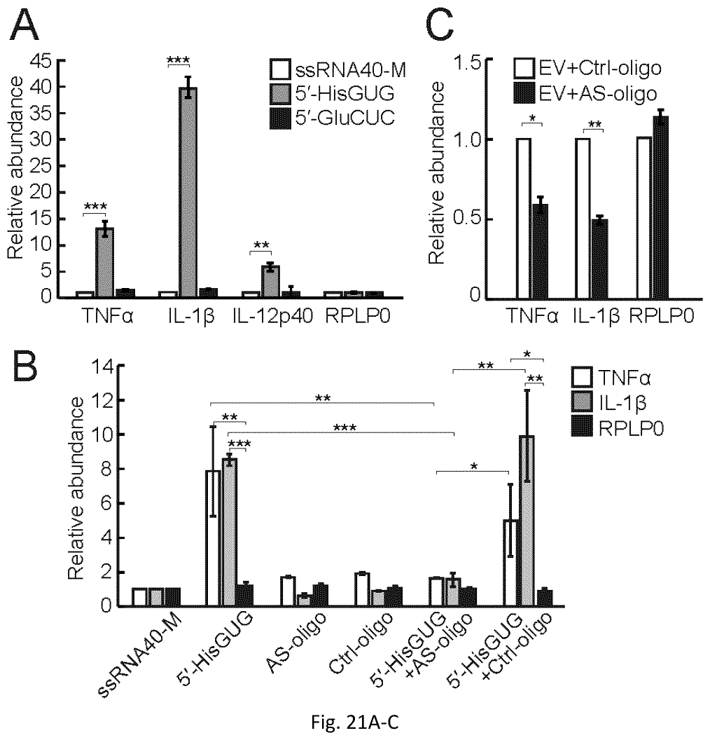

To test whether the EV-5′-tRNA^{HisGUG} half activates TLR7, the 5′-tRNA^{HisGUG} half, 5′-tRNA^{GluCUC} half, and ssRNA40-M (negative control) were transfected into HMDMs, and the EVs isolated from the cells were applied to recipient HMDMs. As shown in FIG. 21A, EVs isolated from HMDMs that transiently expressed the 5′-tRNA^{HisGUG} half were able to induce immune response. To further confirm the activity of endogenous EV-5′-tRNA^{HisGUG} halves, antisense oligonucleotides of the 5′-tRNA^{HisGUG} half and control oligonucleotides with scrambled sequences were utilized. In a DOTAP transfection experiment, both oligonucleotides did not show activity for endosomal TLR by themselves (FIG. 21B). When mixed with an equal amount of 5′-tRNA^{HisGUG} half, the antisense oligonucleotides impaired TLR7 activation by the 5′-tRNA^{HisGUG} half but the control oligonucleotides did not (FIG. 21B), confirming the antisense oligonucleotides' activity to block the 5′-tRNA^{HisGUG} half. In the experiment using the EVs isolated from HMDMs, strikingly, the antisense oligonucleotides of the 5′-tRNA^{HisGUG} half reduced the EV-induced upregulation of TNFα and IL-1β by 40-60% (FIG. 21C). Taken together, these results confirmed that endogenous 5′-tRNA^{HisGUG} halves, which are transferred from EVs to recipient cells, have activity to promote cytokine productions by stimulating endosomal TLR7.

Levels of Circulating 5′-tRNA Halves are Elevated in the Plasma of Mtb-Infected Patients

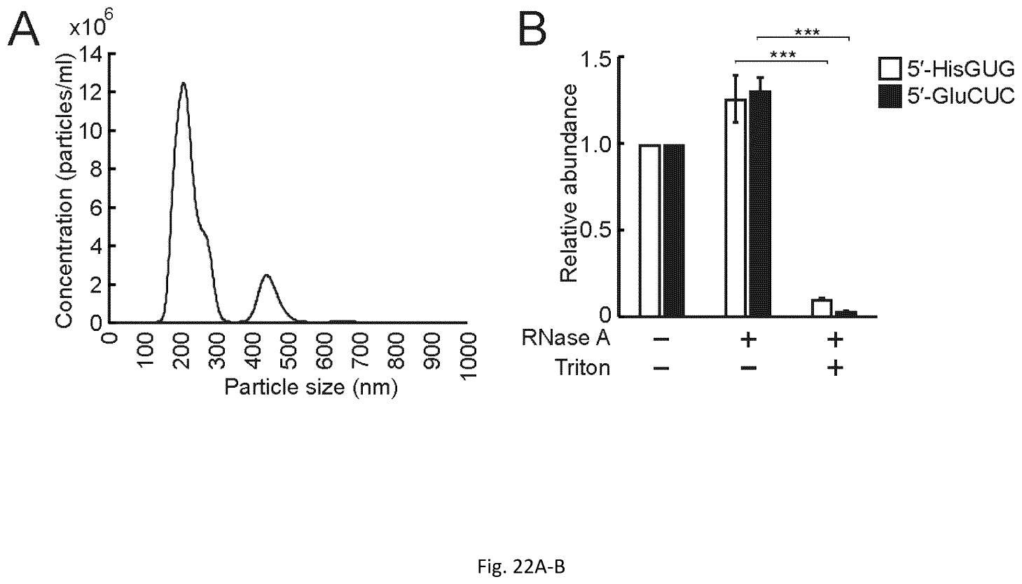

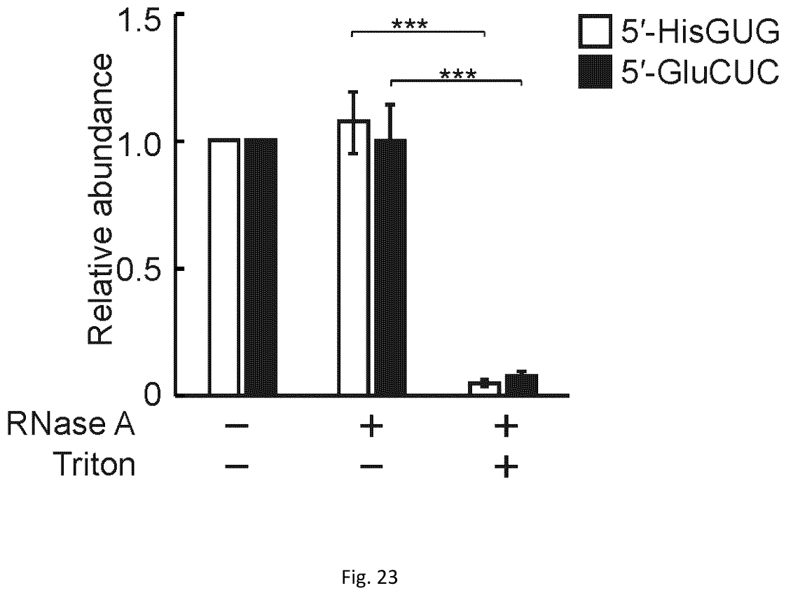

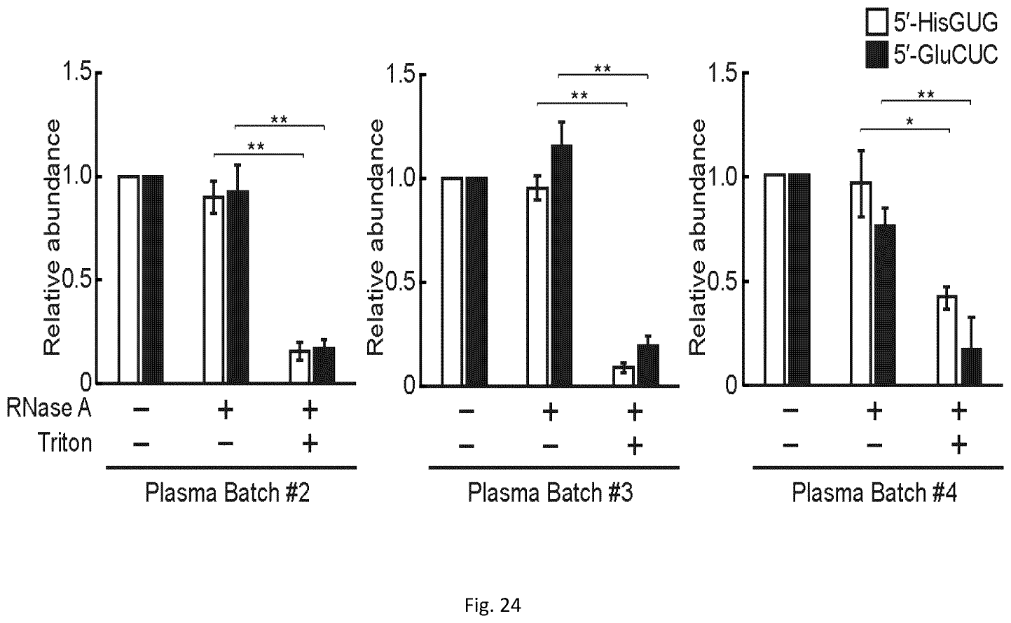

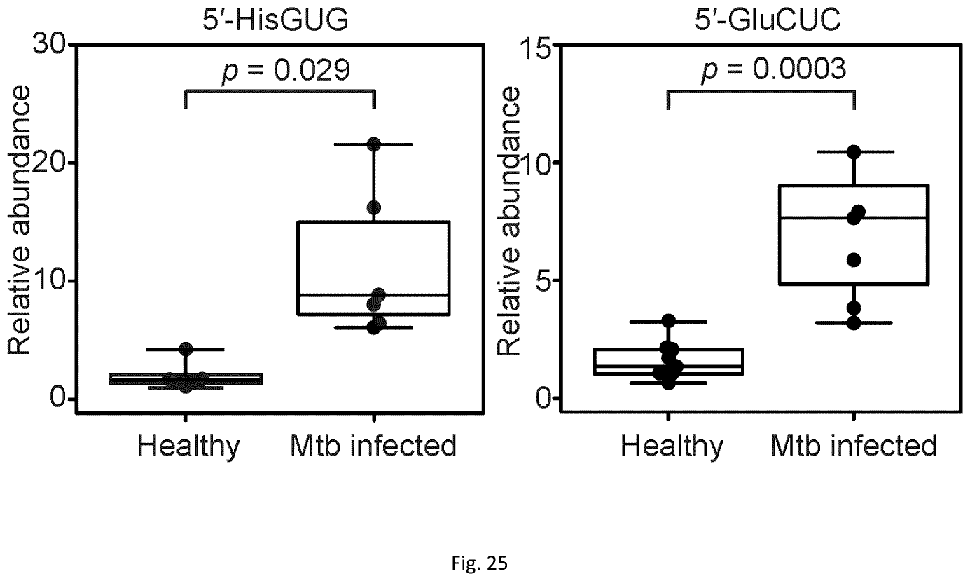

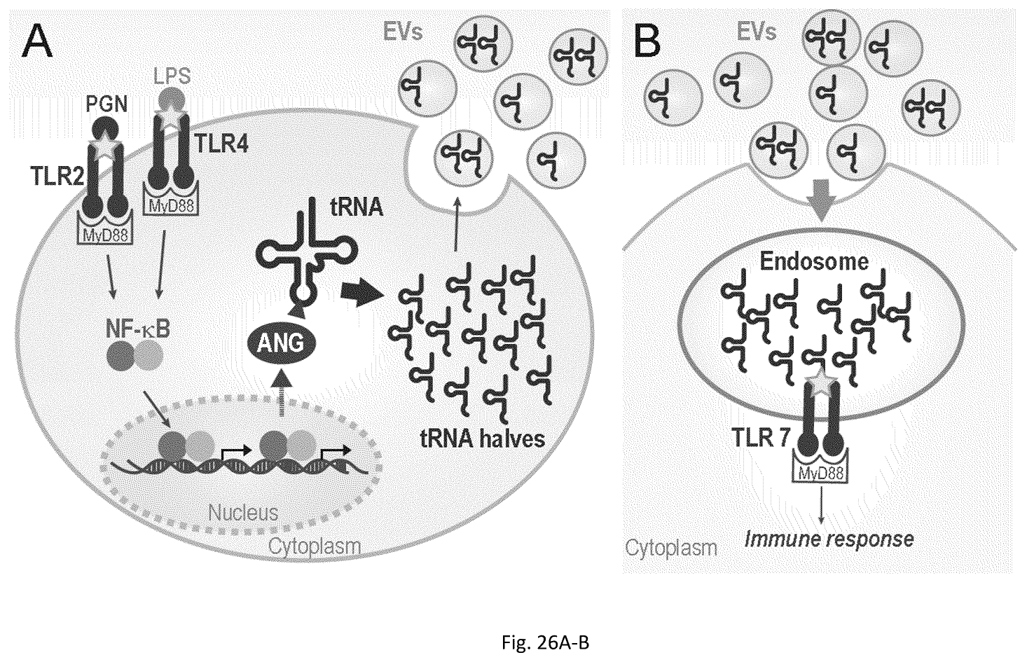

Further, 5′-tRNA half expression in human plasma samples was examined. Plasma EVs were isolated (FIG. 22A) and subjected to treatments with RNase in the presence or absence of detergent. While the plasma EVs treated with RNase alone yielded similar amplification signals to untreated EVs, the EVs treated with both RNase and detergent yielded drastically reduced amplification signals (FIG. 22B), confirming the presence of 5′-tRNA halves inside the plasma EVs. Because the quantification of 5′-tRNA halves using plasma RNAs showed similar amplification patterns with no changes in the levels of 5′-tRNA halves upon RNase treatment of plasma (FIG. 23 and FIG. 24), the detected 5′-tRNA halves in plasma samples were expected to be mostly present inside plasma EVs. The 5′-tRNA halves in the plasma samples from healthy individuals or Mtb-infected patients were then quantified. Because the expression of tRNA halves can be affected by sex hormones (Honda S, et al., 2015, Proc Natl Acad Sci USA, 112: E3816-3825) and aging (Shigematsu M, et al., 2019, PLoS Genet, 15: e1008469), the examined subjects were limited to males aged 30-35 years. During RNA extraction, a synthetic mouse piRNA was added as a spike-in control, and its abundance was used for normalization. As shown in FIG. 25, the expression levels of two examined 5′-tRNA halves were markedly enhanced in Mtb-infected patients compared to healthy subjects. The 5′-tRNA^{HisGUG} half in particular was highly elevated at approximately 10-fold higher in Mtb-infected patients than in control subjects. These results suggest that the upregulation and secretion of 5′-tRNA halves upon infection are not limited to cell culture settings but also occur in actual pathological situations in pathogenic microbe-infected patients.

The conclusions of the present Example are now described herein.

A novel role of 5′-tRNA halves as activators of TLR7 was identified herein. Both BCG infection and PAMP-mediated surface TLR activation induced the expression of 5′-tRNA halves in HMDMs. Considering the results of earlier studies on the function of 5′-tRNA halves in the stress response, translation, and cell proliferation (Honda S, et al., 2015, Proc Natl Acad Sci USA, 112: E3816-3825; Emara M M, et al., 2010, J Biol Chem, 285: 10959-10968; Ivanov P, et al., 2011, Molecular Cell, 43: 613-623; Lyons S M, et al., 2016, Nucleic Acids Res, 44: 6949-6960), infection-induced 5′-tRNA halves could function in various biological processes inside macrophages. In the present study, the secretion of 5′-tRNA halves into EVs and their role as stimulators of endosomal TLRs in recipient cells was the primary focus. Strikingly, the analyses revealed the abundant accumulation of 5′-tRNA halves in HMDM-secreted EVs and their delivery to endosomes in recipient cells for the activation of TLR7. The model proposed herein suggests that infection-induced 5′-tRNA halves function as “immune activators” by being delivered to endosomes in surrounding cells via EV-mediated cell-cell communication and by activating TLR7 (FIG. 26A-B).

Previous studies have shown that stress stimuli and sex hormone signaling pathways induce ANG-catalyzed cleavage of the anticodon-loop of tRNAs, leading to the expression of tRNA halves termed tRNA-derived stress-induced RNAs (tiRNAs) and sex hormone-dependent tRNA-derived RNAs (SHOT-RNAs), respectively (Fu H, et al., 2009, FEBS Lett, 583: 437-442; Yamasaki S, et al., 2009, J Cell Biol, 185: 35-42; Honda S, et al., 2015, Proc Natl Acad Sci USA, 112: E3816-3825). In tiRNA biogenesis, tRNA cleavage is triggered by decreased levels of RNH1, an ANG inhibitor, which increase ANG availability for tRNA cleavage (Saikia M, et al., 2015, J Biol Chem, 290: 29761-29768). Although the mechanism of SHOT-RNA biogenesis is unknown, estrogen or androgen receptors, functioning as transcription factors, might regulate the expression of ANG and/or RNH1. In the case of infection-induced tRNA halves, the analyses herein revealed that TLR-activated NF-cB upregulates the expression levels of ANG mRNA, potentially leading to enhanced levels of ANG protein available for tRNA cleavage. If this is the mechanism behind tRNA half production, because not only surface TLR pathways but also the TLR7 pathway culminates in NF-fB activation, there could be a feed-forward loop in which TLR7 activation by 5′-tRNA halves induces the expression of 5′-tRNA halves for further activation of TLR7. In addition, because dysregulation of NF-κB is linked to various diseases, such as cancers and inflammatory and autoimmune diseases (Xia Y, et al., 2014, Cancer Immunol Res, 2: 823-830; Sun S C, et al., 2013, Trends Immunol, 34: 282-289; Zhang Q, et al., 2017, Cell, 168: 37-57), the potential regulation of tRNA half production by NF-fB suggests the involvement of tRNA halves in such diseases.

By using cP-RNA-seq, the complete expression repertories of 5′-tRNA halves in HMDMs and their secreted EVs were identified, revealing that only specific tRNA species serve as major substrates for infection-induced tRNA half expression. The molecular mechanism underlying the anticodon-loop cleavage of specific tRNA species remains unknown. Because major substrate tRNAs such as cyto tRNA^{ValCAC}, tRNA^{ValAAC}, tRNA^{GlyGCC}, tRNA^{HisGUG}, and tRNA^{GluCUC} were also identified as major sources of SHOT-RNAs in human breast cancer cells (Honda S, et al., 2015, Proc Natl Acad Sci USA, 112: E3816-3825), those tRNAs may be universally susceptible to ANG cleavage, or the molecular factors determining the susceptibility of tRNAs to anticodon cleavage, such as tRNA modifications, may be regulated similarly between the biogenesis of sex-hormone- and infection-induced tRNA halves. The difference in the expression profiles of 5′-tRNA halves between HMDMs and their secreted EVs suggests selective packaging of 5′-tRNA halves into EVs. Selective packaging of the 5′-tRNA^{HisGUG} half into EVs is intriguing as this half is highly active in TLR7 stimulation. Although the mechanism of EV RNA content selection is unknown, biased EV incorporation has been also shown for miRNAs (Villarroya-Beltri C, et al., 2013, Nat Commun, 4: 2980; Santangelo L, et al., 2016, Cell Rep, 17: 799-808; Shurtleff M J, et al., 2016, Elife, 5: e19276) and tRFs (Chiou N T, et al., 2018, Cell Rep, 25(12): 3356-3370; Goodarzi H, et al., 2015, Cell, 161: 790-802). Because Y-box protein 1 (YBX1) has been reported to interact with 5′-tRNA halves (Ivanov P, et al., 2011, Molecular Cell, 43: 613-623) and has been implicated in the sorting of miRNAs for packaging into EVs (Shurtleff M J, et al., 2016, Elife, 5: e19276), such RNA-binding proteins could be involved in the selective packaging of 5′-tRNA halves. Among the cellular 5′-tRNA^{HisGUG} half species, only a specific 5′-tRNA^{HisGUG} half, from G₁ to G₃₄, is preferentially packaged into EVs. Specific sequences and/or secondary/tertiary structures may contribute to preferential binding to RNA-binding proteins responsible for EV packaging. Indeed, in the case of miRNAs, specific 3′-terminal sequences are required to interact with heterogeneous nuclear ribonucleoprotein A2/B1 for preferential incorporation into EVs (Villarroya-Beltri C, et al., 2013, Nat Commun, 4: 2980).

One of the most remarkable characteristics of 5′-tRNA halves is their abundance. Although miR-150 was identified as one of the most abundant miRNAs in HMDMs and their EVs (Zhang Y, et al., 2010, Mol Cell, 39: 133-144), the present quantification revealed the abundance of the 5′-tRNA^{HisGUG} half in HMDMs and EVs to be over 130-fold and 210-fold higher, respectively. Although miRNAs have been shown to function as ligands for TLR7, considering ligand-receptor interactions, 5′-tRNA halves with much more abundance could be more efficient, superior TLR ligands than miRNAs. Given that T4 PNK treatment greatly enhanced amounts of EV-cDNAs during the described sequencing procedure, it is predicted that EV-short ncRNA species are mostly 3′-P- or cP-containing RNAs, such as 5′-tRNA halves, and that 3′-OH-containing RNAs, such as miRNAs, are minor species. While studies on EVs have established the role of EV-RNAs as cell-cell communication agents (Tkach M, et al., 2016, Cell, 164: 1226-1232), most current studies rely on standard RNA-seq, which cannot capture the 3′-P or cP-containing RNAs that account for the majority of short RNA species in EVs. The results presented herein suggest the necessity of shedding light on these previously-unrecognized RNAs by pre-treating EV-RNA fractions with T4 PNK in sequencing studies. Giraldez et al. revealed previously-unexplored mRNA and lncRNA fragments by phosphor-RNA-seq whose procedure includes T4 PNK treatment (Giraldez M D, et al., 2019, EMBO J, 38(11): e101695).

Another striking feature of the 5′-tRNA^{HisGUG} half is its ability to strongly activate TLR7, but not TLR8. This selective activity for TLR7 might result from the high sensitivity of TLR7 to GU-rich ssRNAs, such as the 5′-tRNA^{HisGUG} half, while TLR8 senses AU-rich ssRNAs (Forsbach A, et al., 2008, J Immunol, 180: 3729-3738). The activation of TLR7 by the 5′-tRNA^{HisGUG} half is as high as that by SSRNA40, suggesting the role of the 5′-tRNA^{HisGUG} half as an endogenous ligand for TLR7 with the full capacity to produce an immune response. On the other hand, the 5′-tRNA^{GluCUC} half did not activate TLR7. Because the 5′-tRNA^{GluCUC} half and the 5′-tRNA^{HisGUG} half were similarly delivered to recipient endosomes in our delivery experiments, the inactivity of the 5′-tRNA^{GluCUC} half is probably due to its inefficient binding to TLR7. The lack of 3′-terminal GU-rich sequences may be one of the reasons for the inefficient activity of 5′-tRNA^{GluCUC} toward TLR7 as previous study showed significance of 3′-terminal GU sequences in let-7 miRNA for TLR7 activation (Lehmann S M, et al., 2012, Nat Neurosci, 15: 827-835). Intriguingly, unlike the 5′-tRNA^{HisGUG} half, the full-length tRNA^{HisGUG} is incapable of activating TLR7, suggesting the cruciality of tRNA cleavage and production of tRNA half molecules to yield active ligands for TLR7.

Finally, the elevation of 5′-tRNA half levels in the plasma of Mtb-infected patients was showed, demonstrating the expressional induction of secretion of 5′-tRNA halves in actual pathological situations. Because upregulation of 5′-tRNA half expression has been reported upon infection with respiratory syncytial virus (Zhou J, et al., 2017, J Gen Virol, 98: 1600-1610; Deng J, et al., 2015, Mol Ther, 23: 1622-1629), Rickettsia (Gong B, et al., 2013, BMC Infect Dis, 13(1): 285), and hepatitis B and C viruses (Selitsky S R, et al., 2015, Sci Rep, 5: 7675), induction of 5′-tRNA halves could be a universal phenomenon among infectious diseases. Considering the expressional differences and the demonstrated roles of 5′-tRNA halves in the innate immune response, this suggests the use of 5′-tRNA halves as potential target candidates for future therapeutic applications and/or circulating biomarkers for noninvasive testing to estimate the severity of infectious diseases and the status of the immune response.

### Example 2: Function of 5′-Half Molecules of tRNA^{HisGUG} and 5′-tRNA^{ValCAC/AAC} as an Immunity Booster

Toll-Like Receptors (TLRs) play a key role in the immune response by protecting the host body against invading pathogens or endogenously released hazardous molecules. Endosomal TLR7 and -8 are primarily expressed in immune cells such as monocytes/macrophages, dendritic cells, neutrophils, and B cells, and sense single-stranded RNAs (ssRNAs) as their ligands (Barton G M, et al., 2009, Nat Rev Immunol, 9: 535-542; Blasius, A L et al., 2010, Immunity, 32: 305-315). Their ssRNA recognition recruits MyD88, activates NF-κB-mediated transcription, and induces the production of interferons and cytokines (Heil F, et al., 2004, Science, 303(5663): 1526-1529; Saha B, et al., 2017, J Immunology, 198(5): 1974-1984).

In Example 1 described above, it was shown that specific 5′-tRNA half molecules expressed in macrophages (HMDMs) are packaged into extracellular vehicles (EVs) and are then delivered into endosomes in recipient cells where they activate endosomal TLR7. Regarding TLR7 activation, Example 1 focused on 5′-tRNA^{HisGUG} half. However, as described, we identified various 5′-tRNA half species expressed in HMDM EVs, and we investigated whether those species, in addition to 5′-tRNA^{HisGUG} half, also activate TLR7.

The methods of the present Example are now described herein.

Cell and Bacterial Cultures

THP-1 human acute monocytic leukemia cells (American Type Culture Collection) were cultured in RPMI 1640 medium (Corning) and differentiated into human monocyte-derived macrophages (HMDMs) using phorbol 12-myristate 13-acetate (PMA; Sigma-Aldrich) as described previously (Pawar K, et al., 2016, Front Cell Infect Microbiol, 6: 27; Pawar K, et al., 2016, Sci Rep, 6: 19416). The E. co/i stain K-12 (American Type Culture Collection) were grown as previously described (Mackie A, et al., 2014, J Bacteriol, 196(5): 982-988).

DOTAP-Mediated RNA Delivery to Endosomes

To deliver RNAs to endosomes, DOTAP liposomal transfection reagent (Sigma-Aldrich) was used as previously described (Fabbri M, et al., 2012, Proc Natl Acad Sci USA, 109: E2110-2116; Honda K, et al., 2005, Nature, 434(7036): 1035-40). In brief, 230 pmol or other various amounts of synthetic RNAs were mixed with 60 μl of HBS buffer and 15 μl of DOTAP reagent and incubated for 15 min. The RNA-DOTAP solution was then added to 1 ml of HMDM culture medium, followed by incubation of the cells for 16 h. The supernatant was collected and used for measurement of cytokines.

Measurement of Cytokines

The content of cytokines in supernatant was measured by Multiplexing LASER Bead Technology (Eve Technologies) using a Human Cytokine Array Proinflammatory Focused 15-plex (Cat #: HDF15).

Infection/Invasion Assay

HMDMs (1×10⁶ cells) were plated on 6-well plates and incubated with E. co/i in RPMI 1640 (no antibiotics) for 60 min. Two different amounts of bacteria, 10⁷ and 10⁸ cells, were used as final inocula to obtain a multiplicity of infection (MOI) ratio of 10 and 100, respectively. HMDMs were then washed with PBS and incubated with RPMI 1640 containing high concentration (3×) antibiotics for 60 min, followed by further incubation in normal RPMI 1640 medium for 24 hours. After wash and lyse of the HMDMs, the intracellular bacteria were enumerated by plating preparations on LB agar plates.

Other Methods

The remaining methods are described in the methods of Example 1 above.

The results of the present Example are now described herein.

Only Specific 5′-tRNA Halves were Identified as Major Species Present in Macrophage EVs

The above described sequencing data revealed whole 5′-tRNA half species accumulated in EVs secreted from HMDMs (Example 1). Considering that the human genome encodes 55 cytoplasmic (cyto) tRNA isoacceptors with different anticodon sequences, the identified 5′-tRNA halves were derived from a rather focused subset of tRNAs, such as cyto tRNA^{ValCAC}, tRNA^{ValAAC}, tRNA^{GlyGCC}, tRNA^{HisGUG}, and tRNA^{GluCUC}, which are in aggregates the sources of 88-90% of the identified 5′-tRNA halves in EVs. When the 5′-tRNA half species whose read numbers were >1% of total tRNA-mapped reads were focused on, there were 16 5′-tRNA halves. Because 5′-tRNA^{GluCUC} half has been already shown to be inactive in TLR7 simulation (Example 1), the most abundant species from tRNA^{ValCAC}, tRNA^{HisGUG}, tRNA^{GlyGCC}, and tRNA^{ValAAC} were the focus of continued study in Example 2.

Not Only 5′-tRNA^{HisGUG} Half but Also 5′-tRNA^{ValCAC/ValAAC} Halves Functionally Activate Endosomal TLR7 for Cytokine Secretion

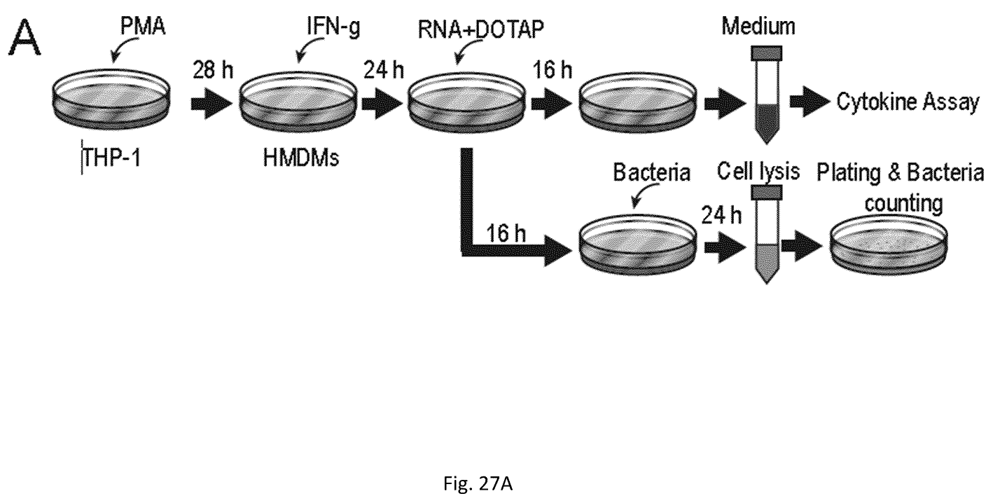

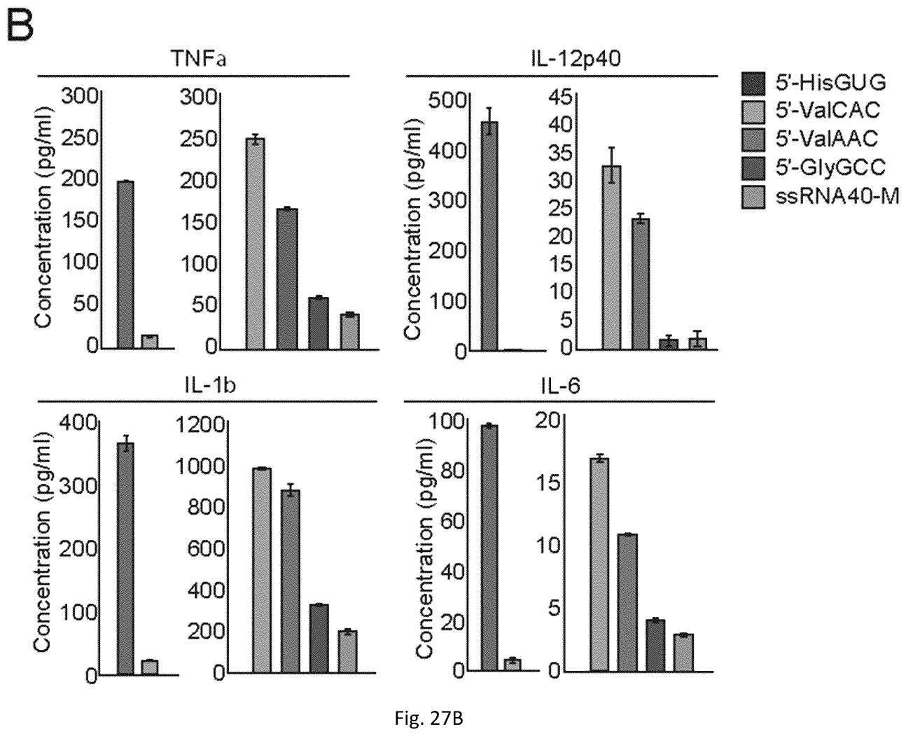

To investigate the activity of the four abundant 5′-tRNA half species, as described in earlier studies (Gantier M P, et al., 2008, J Immunol, 180: 2117-2124; Hamidzadeh K, et al., 2016, Frontiers in Immuno, 7:74; Hipp M M, et al., 2013, Immunity, 39(4): 711-21), HMDMs were primed with interferon γ and then transfected with the selected 5′-tRNA half using the cationic liposome 1,2-dioleoyloxy-3-trimethylammonium-propane (DOTAP) which mimics EVs (FIG. 27A). As a negative control, ssRNA40-M, (in which U is replaced with A of 20-nt HIV-1-derived ssRNA termed ssRNA40; Heil F, et al., 2004, Science. 303: 1526-1529) was also transfected. As shown in FIG. 27B, transfections of the 5′-tRNA^{HisGUG} half, 5′-tRNA^{ValCAC} half, and 5′-tRNA^{ValAAC} half increased the secretion of various pro-inflammatory cytokines, whereas transfections of the 5′-tRNA^{GlyGCC} half and ssRNA40-M (a negative control) did not. These results suggest that 5′-tRNA^{ValCAC/ValAAC} halves, as well as a 5′-tRNA^{HisGUG} half (already shown in Example 1 above), functionally activate endosomal TLR7 for cytokine secretion.

Activation of Endosomal TLR7 by 5′-tRNA Halves Prior to the Infection Enhances Bacterial Elimination

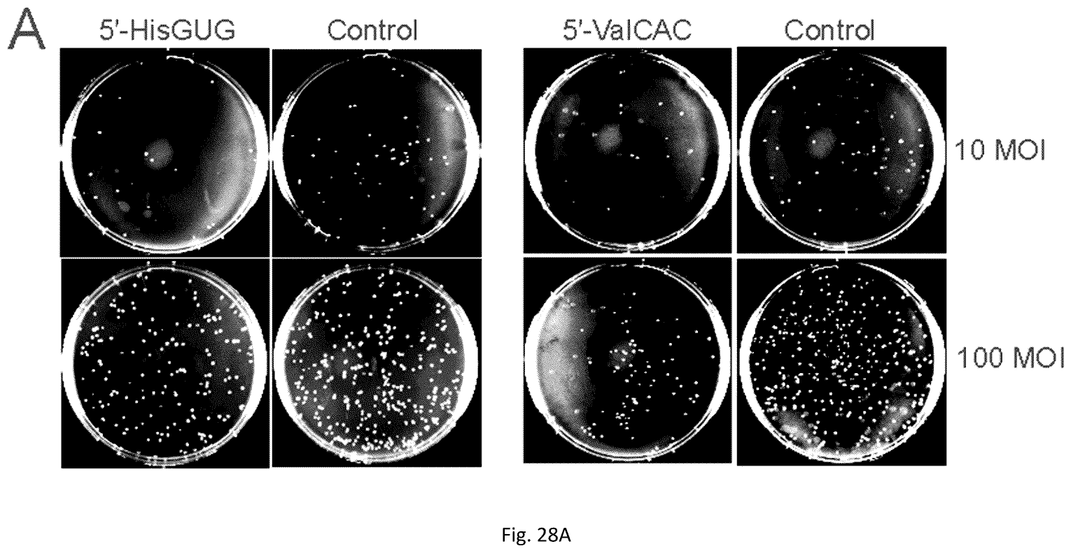

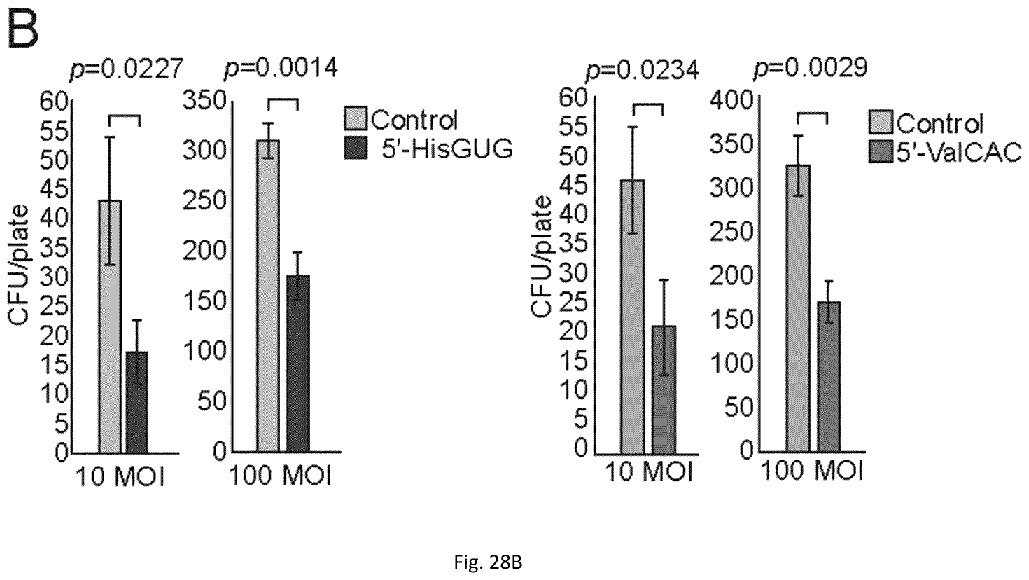

After 5′-tRNA^{HisGUG} half- or 5′-tRNA^{ValCAC} half-mediated TLR activation, HMDMs were infected with E-coli with different multiplicities of infection (MOI) and incubated (FIG. 27A). HMDMs were then lysed and plated on LB agar plates. Representative plate images after incubation are shown in FIG. 28A, and colony forming units (CFU) per plate were counted. As shown in FIG. 28B, CFU were significantly reduced by the transfection of 5′-tRNA^{HisGUG} half or 5′-tRNA^{ValCAC} half molecules, indicating that those 5′-tRNA halves act as immune boosters to actually eliminate bacteria.

The conclusions of the present Example are now described herein.

Because of its role in the immunity, TLR7/8 agonists can be used for the immunotherapy, adjuvant strategy, antiviral/antibacterial action, and treatments of allergy and asthma (Patinote C, et al., 2020, Eur J Med Chem, 112238). Many pharmaceutical companies and research institutions are thereby developing specific TLR modulators. Some of them have been pre-clinically and clinically evaluated. One of the commonly and widely identified synthetic agonists for TLR7/8 are imidazoquinolines (Shi C, et al., 2012, ACS Med Chem Ltrs, 3(6): 501-504). Derivatives of imidazoquinolines, including imiquimod and resiquimod [R-848], are low molecular weight synthetic compounds that have potent immune stimulating properties and have demonstrated anti-viral and anti-tumor activity (Miller R L, et al., 1999, Int J Immunopharmacol, 21: 1-14; Chen M, et al., 1988, Antimicrob Agents Chemother, 32: 678-683; Bernstein D I, et al., 2001, J Infect Dis, 183: 844-849; Stanley M A, et al., 2002, Clin Exp Dermatol, 27: 571-577). However, those synthetic compounds are not perfect. For example, R-848 has a poor tolerability profile when tested in humans, and common systemic side effects include injection site reactogenicity and flu-like symptoms (fever, headache, and malaise) that correlate with systemic immune activation (Sauder D N, et al., 2003, Antimicrob Agents Chemother, 47: 3846-3852; Szeimies R M, et al., 2008, Br J Dermatol, 159: 205-210). The molecule 852A (a. k. a. S-32865: N-[4-(4-amino-2-ethyl-1H-imidazo[4,5-c]quinolin-1-yl)butyl]methanesulfonamide) has only moderate bioavailability of 26.5±7.84% and also has highly variable absorption and lower availability in oral dosing compared with intravenous and subcutaneous injection.

The natural ligands for TLR7/8 in the cells are ssRNAs, and therefore, ssRNAs may function as superior TLR7/8 modulators with lower cellular toxicity compared to synthetic compounds for the immunotherapy and other therapeutic applications. Herein it was experimentally demonstrated that 5′-tRNA^{HisGUG} half, 5′-tRNA^{ValCAC} half, and 5′-tRNA^{ValAAC} half, naturally expressed RNAs in human body, act as a potent agonist for the TLR7, and that their TLR7 activation actually eliminates bacteria. Together with the data presented in Example 1 above, these results suggest that tRNA half molecules can be used as immune boosters in various therapeutic applications. To improve stability inside the human body, covalent linkage of two ssRNA species by their 3′-ends can be used or the 5′ guanosine nucleotides can be substituted with nuclease-resistant 7-deazaguanosines (Lan T, et al., 2007, Proc Natl Acad Sci USA, 104: 13750-13755). Moreover, use of protamine to condense RNA that serves to protect nucleic acids from degradation (Miller D, et al., 2010, Reproduction, 139: 287-301) is also an effective agent to improve efficacy in vivo. By applying those technical improvements, it is believed that tRNA half molecules will act as novel and stable immune boosters in various therapeutic applications.

### Example 3: Sequences

| Name | tRNA | Sequence | Length |
|---|---|---|---|
| tRH-#1 | ValCAC | GUUUCCGUAGUGUAGUGGUUAUCACGUUCGCCU (SEQ | 33 |
| | | ID NO: 57) | |
| tRH-#2 | HisGUG | GCCGUGAUCGUAUAGUGGUUAGUACUCUGCGUUG | 34 |
| | | (SEQ ID NO: 1) | |
| tRH-#3 | GlyGCC | GCAUUGGUGGUUCAGUGGUAGAAUUCUCGCCU (SEQ | 32 |
| | | ID NO: 58) | |
| tRH-#4 | GluCUC | UCCCUGGUGGUCUAGUGGUUAGGAUUCGGCGCUCUC | 36 |
| | | (SEQ ID NO: 59) | |
| tRH-#5 | ValAAC | GUUUCCGUAGUGUAGUGGUCAUCACGUUCGCCU (SEQ | 33 |
| | | ID NO: 60) | |
| tRH-#6 | GluUUC | UCCCACAUGGUCUAGCGGUUAGGAUUCCUGGUU (SEQ | 33 |
| | | ID NO: 61) | |
| tRH-#7 | LysCUU | GCCCGGCUAGCUCAGUCGGUAGAGCAUGAGACU (SEQ | 33 |
| | | ID NO: 62) | |
| tRH-#8 | AspGUC | UCCUCGUUAGUAUAGUGGUGAGUAUCCCCGCCUGUC | 36 |
| | | (SEQ ID NO: 63) | |
| tRH-#9 | MetCAU | AGCAGAGUGGCGCAGCGGAAGCGUGCUGGGCCC (SEQ | 33 |
| | | ID NO: 64) | |
| tRH-#10 | ProAGG | GGCUCGUUGGUCUAGGGGUAUGAUUCUCGCUU (SEQ | 32 |
| | | ID NO: 65) | |
| tRH-#11 | LeuCAG | GUCAGGAUGGCCGAGCGGUCUAAGGCGCUGCGUUC | 35 |
| | | (SEQ ID NO: 66) | |
| RH-#12 | ArgUCU | GGCUCCGUGGCGCAAUGGAUAGCGCAUUGGACU (SEQ | 33 |
| | | ID NO: 67) | |
| tRH-#13 | LysUUU | GCCCGGAUAGCUCAGUCGGUAGAGCAUCAGACU (SEQ | 33 |
| | | ID NO: 68) | |
| tRH-#14 | ValUAC | GGUUCCAUAGUGUAGUGGUUAUCACGUCUGCUUU | 34 |
| | | (SEQ ID NO: 69) | |
| tRH-#15 | GlnCUG | GGUUCCAUGGUGUAAUGGUUAGCACUCUGGACUC | 34 |
| | | (SEQ ID NO: 70) | |
| tRH-#16 | ArgCCG | GACCCAGUGGCCUAAUGGAUAAGGCAUCAGCCU (SEQ | 33 |
| | | ID NO: 71) | |
| tRH-#17 | ArgACG | GGGCCAGUGGCGCAAUGGAUAACGCGUCUGACU (SEQ | 33 |
| | | ID NO: 72) | |
| tRH-#18 | LeuUAA | GUUAAGAUGGCAGAGCCUGGUAAUUGCAUAAAACUUA | 37 |
| | | (SEQ ID NO: 73) | |
| tRH-#19 | ArgUCG | GACCGCGUGGCCUAAUGGAUAAGGCGUCUGACU (SEQ | 33 |
| | | ID NO: 74) | |
| tRH-#20 | AsnGUU | GUCUCUGUGGCGCAAUCGGUUAGCGCAUUCGGCU | 34 |
| | | (SEQ ID NO: 75) | |
| tRH-#21 | AlaCGC | GGGGAUGUAGCUCAGUGGUAGAGCGCGCGCUU (SEQ | 32 |
| | | ID NO: 76) | |
| tRH-#22 | LeuAAG | GGUAGCGUGGCCGAGCGGUCUAAGGCGCUGGAUU | 34 |
| | | (SEQ ID NO: 77) | |
| tRH-#23 | ThrUGU | GGCUCCAUAGCUCAGGGGUUAGAGCACUGGUC (SEQ | 32 |
| | | ID NO: 78) | |
| tRH-#24 | AlaAGC | GGGGAAUUAGCUCAAAUGGUAGAGCGCUCGCUU (SEQ | 33 |
| | | ID NO: 79) | |
| tRH-#25 | LeuCAA | GUCAGGAUGGCCGAGUGGUCUAAGGCGCCAGAC (SEQ | 33 |
| | | ID NO: 80) | |
| tRH-#26 | PheGAA | GCCGAAAUAGCUCAGUUGGGAGAGCGUUAGACU (SEQ | 33 |
| | | ID NO: 81) | |
| tRH-#27 | GlnUUG | GGCCCCAUGGUGUAAUGGUUAGCACUCUGGACU (SEQ | 33 |
| | | ID NO: 82) | |
| tRH-#28 | TrpCCA | GGCCUCGUGGCGCAACGGUAGCGCGUCUGACUCC (SEQ | 34 |
| | | ID NO: 83) | |
| tRH-#29 | SerGCU | GACGAGGUGGCCGAGUGGUUAAGGCGAUGGACUGCU | 36 |
| | | (SEQ ID NO: 84) | |
| tRH-#30 | CysGCA | GGGGGUAUAGCUCAGUGGGUAGAGCAUUUGACU (SEQ | 33 |
| | | ID NO: 85) | |
| tRH-#31 | IleAAU | GGCCGGUUAGCUCAGUCGGCUAGAGCGUGGUGCUA | 35 |
| | | (SEQ ID NO: 86) | |
| tRH-#32 | ArgCCU | GCCCCAGUGGCCUAAUGGAUAAGGCAUUGGCCU (SEQ | 33 |
| | | ID NO: 87) | |
| tRH-#33 | SerAGA | GUAGUCGUGGCCGAGUGGUUAAGGCGAUGGAC (SEQ | 32 |
| | | ID NO: 88) | |
| tRH-#34 | GlyUCC | GCGUUGGUGGUAUAGUGGUUAGCAUAGCUGCCU (SEQ | 33 |
| | | ID NO: 89) | |
| tRH-#35 | LeuUAG | GGUAGUGUGGCCGAGCGGUCUAAGGCGCUGGAUU | 34 |
| | | (SEQ ID NO: 90) | |
| tRH-#36 | AlaUGC | GGGGAUGUAGCUCAGUGGUAGAGCGCAUGCUU (SEQ | 32 |
| | | ID NO: 91) | |
| tRH-#37 | ThrCGU | GGCAGAGUGGUGCAGCGGAAGCGUGCUGGGCCC (SEQ | 33 |
| | | ID NO: 92) | |

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of treating or preventing a disease or disorder in a subject in need thereof, the method comprising administering a nucleic acid molecule comprising a fragment or variant of a tRNA molecule to the subject.

2. The method of claim 1, wherein the fragment or variant of a tRNA molecule activates at least one toll-like receptor (TLR).

3. The method of claim 2, wherein the TLR is selected from the group consisting of TLR7, TLR8 and a combination thereof.

4. The method of claim 1, wherein the fragment or variant of a tRNA molecule comprises a fragment comprising at least 4 nucleotides of a tRNA molecule.

5. The method molecule of claim 1, wherein the tRNA is selected from the group consisting of tRNA^{HisGUG}, tRNA^{GluCUC}, tRNA^{ValCAC}, tRNA^{GlyGCC}, tRNA^{ValAAC}, tRNA^{GluUUC}, tRNA^{LysCUU}, tRNA^{AspGUC}, tRNA^{MetCAU}, tRNA^{ProAGG}, tRNA^{LeuCAG}, tRNA^{ArgUCU}, tRNA^{LysUUU}, tRNA^{ValUAC}, tRNA^{GlnCUG}, tRNA^{ArgCCG}, tRNA^{ArgACG}, tRNA^{LeuUAA}, tRNA^{ArgUCG}, tRNA^{AsnGUU}, tRNA^{AlaCGC}, tRNA^{LeuAAG}, tRNA^{ThrUGU}, tRNA^{AlaAGC}, tRNA^{LeuCAA}, tRNA^{PheGAA}, tRNA^{GlnUUG}, tRNA^{TrpCCA}, tRNA^{SerGCU}, tRNA^{CysGCA}, tRNA^{IleAAU}, tRNA^{ArgCCU}, tRNA^{SerAGA}, tRNA^{GlyUCC}, tRNA^{LeuUAG}, tRNA^{AlaUGC}, and tRNA^{ThrCGU}.

6. The method of claim 5, wherein the tRNA is selected from the group consisting of tRNA^{HisGUG}, tRNA^{ValCAC}, and tRNA^{ValAAC}.

7. The method of claim 1, wherein the fragment or variant of a tRNA molecule comprises at least one selected from the group consisting of:
a) an RNA molecule comprising a nucleotide sequence selected from the group consisting of SEQ ID NO:1, SEQ ID NO:3, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91 and SEQ ID NO:92;
b) a fragment of an RNA molecule comprising a nucleotide sequence selected from the group consisting of SEQ ID NO: 1, SEQ ID NO:3, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91 and SEQ ID NO:92 comprising at least 4 nucleotides;
c) a variant of an RNA molecule comprising a nucleotide sequence selected from the group consisting of SEQ ID NO:1, SEQ ID NO:3, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91 and SEQ ID NO:92 comprising at least one modified nucleotide; and
d) a variant of an RNA molecule comprising a nucleotide sequence selected from the group consisting of SEQ ID NO:1, SEQ ID NO:3, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91 and SEQ ID NO:92 comprising a sequence having at least 90% identity to an RNA molecule comprising a nucleotide sequence selected from the group consisting of SEQ ID NO:1, SEQ ID NO:3, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91 and SEQ ID NO: 92.

8. The method of claim 7, wherein the variant of SEQ ID NO:1 comprises SEQ ID NO:2.

9. The method of claim 1, wherein the disease or disorder is selected from the group consisting of cancer and an infectious disease.

10. A nucleic acid molecule comprising a fragment or variant of a tRNA molecule.

11. The nucleic acid molecule of claim 10, wherein the fragment or variant of a tRNA molecule activates at least one toll-like receptor (TLR).

12. The nucleic acid molecule of claim 10, wherein the TLR is selected from the group consisting of TLR7, TLR8 and a combination thereof.

13. The nucleic acid molecule of claim 10, wherein the fragment or variant of a tRNA molecule comprises a fragment comprising at least 4 nucleotides of a tRNA molecule.

14. The nucleic acid molecule of claim 10, wherein the tRNA is selected from the group consisting of tRNA^{HisGUG}, tRNA^{GluCUC}, tRNA^{ValCAC}, tRNA^{GlyGCC}, tRNA^{ValAAC}, tRNA^{GluUUC}, tRNA^{LysCUU}, tRNA^{AspGUC}, tRNA^{MetCAU}, tRNA^{ProAGG}, tRNA^{LeuCAG}, tRNA^{ArgUCU}, tRNA^{LysUUU}, tRNA^{ValUAC}, tRNA^{GlnCUG}, tRNA^{ArgCCG}, tRNA^{ArgACG}, tRNA^{LeuUAA}, tRNA^{ArgUCG}, tRNA^{AsnGUU}, tRNA^{AlaCGC}, tRNA^{LeuAAG}, tRNA^{ThrUGU}, tRNA^{AlaAGC}, tRNA^{LeuCAA}, tRNA^{PheGAA}, tRNA^{GlnUUG}, tRNA^{TrpCCA}, tRNA^{SerGCU}, tRNA^{CysGCA}, tRNA^{IleAAU}, tRNA^{ArgCCU}, tRNA^{SerAGA}, tRNA^{GlyUCC}, tRNA^{LeuUAG}, tRNA^{AlaUGC}, and tRNA^{ThrCGU}.

15. The nucleic acid molecule of claim 14, wherein the tRNA is selected from the group consisting of tRNA^{HisGUG}, tRNA^{ValCAC}, and tRNA^{ValAAC}.

16. The nucleic acid molecule of claim 10, wherein the fragment or variant of a tRNA molecule comprises at least one selected from the group consisting of:
a) an RNA molecule comprising a nucleotide sequence selected from the group consisting of SEQ ID NO:1, SEQ ID NO:3, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91 and SEQ ID NO:92;
b) a fragment of an RNA molecule comprising a nucleotide sequence selected from the group consisting of SEQ ID NO: 1, SEQ ID NO:3, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91 and SEQ ID NO:92 comprising at least 4 nucleotides;
c) a variant of an RNA molecule comprising a nucleotide sequence selected from the group consisting of SEQ ID NO:1, SEQ ID NO:3, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91 and SEQ ID NO:92 comprising at least one modified nucleotide; and
d) a variant of an RNA molecule comprising a nucleotide sequence selected from the group consisting of SEQ ID NO:1, SEQ ID NO:3, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91 and SEQ ID NO:92 comprising a sequence having at least 90% identity to an RNA molecule comprising a nucleotide sequence selected from the group consisting of SEQ ID NO:1, SEQ ID NO:3, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91 and SEQ ID NO:92.

17. The nucleic acid molecule of claim 16, wherein the variant of SEQ ID NO:1 comprises SEQ ID NO:2.

18. A composition comprising the nucleic acid molecule of claim 10 comprising a fragment or variant of a tRNA molecule.

19. The composition of claim 18, further comprising at least one selected from the group consisting of a pharmaceutically acceptable excipient and an adjuvant.

20. The composition of claim 18, wherein the composition further comprises at least one additional therapeutic agent.

21. The composition of claim 20, wherein the additional therapeutic agent is selected from the group consisting of an altered T-cell, a chimeric antigen receptor T-cell (CAR-T), an antigen, a vaccine, an antibody, an immune checkpoint inhibitor, a small molecule, a chemotherapeutic agent, and a stem cell.

22. A method of increasing an immune response in a subject in need thereof, the method comprising administering the composition of claim 18 to the subject.

23. A method of activating a TLR in a subject in need thereof, the method comprising administering the composition of claim 18 to the subject.

24. The method of claim 23 wherein the TLR is selected from the group consisting of TLR7, TLR8 and a combination thereof.


SEQUENCE LISTING (93 sequences)

SEQ ID NO: 1 - RNA, 34 nt, Artificial Sequence
  Chemically Synthesized
gccgugaucguauagugguuaguacucugcguug

SEQ ID NO: 2 - RNA, 33 nt, Artificial Sequence
  Chemically Synthesized
gccgugaucguauagdggddaguacucugcgug

SEQ ID NO: 3 - RNA, 34 nt, Artificial Sequence
  Chemically Synthesized
ucccugguggucuagugguuaggauucggcgcuc

SEQ ID NO: 4 - RNA, 72 nt, Artificial Sequence
  Chemically Synthesized
gccgugaucguauagugguuaguacucugcguuguggccgcagcaaccucgguucgaauccgagucacggca

SEQ ID NO: 5 - RNA, 20 nt, Artificial Sequence
  Chemically Synthesized
gcccgucuguugugugacuc

SEQ ID NO: 6 - RNA, 20 nt, Artificial Sequence
  Chemically Synthesized
gcccgacagaagagagacac

SEQ ID NO: 7 - RNA, 22 nt, Artificial Sequence
  Chemically Synthesized
uagcuuaucagacugauguuga

SEQ ID NO: 8 - RNA, 22 nt, Artificial Sequence
  Chemically Synthesized
ucucccaacccuuguaccagug

SEQ ID NO: 9 - RNA, 28 nt, Artificial Sequence
  Chemically Synthesized
ugagaguggcaucuaaauguuuaguggu

SEQ ID NO: 10 - DNA, 25 bp, Artificial Sequence
  Chemically Synthesized
gcttaatacgactcactatagccgt

SEQ ID NO: 11 - DNA, 50 bp, Artificial Sequence
  Chemically Synthesized
caacgcagagtactaaccactatacgatcacggctacgatcacggctagt

SEQ ID NO: 12 - DNA, 97 bp, Artificial Sequence
  Chemically Synthesized
cctgcagtaatacgactcactatagggagaagggactgatgagtccgtgaggacgaaacggtacccggtaccgtctccctggtggtctagtggttag

SEQ ID NO: 13 - DNA, 21 bp, Artificial Sequence
  Chemically Synthesized
gagcgccgaatcctaaccact

SEQ ID NO: 14 - DNA, 26 bp, Artificial Sequence
  Chemically Synthesized
gcttaatacgactcactatagcccgt

SEQ ID NO: 15 - DNA, 26 bp, Artificial Sequence
  Chemically Synthesized
gagtcacacaacagacgggctatagt

SEQ ID NO: 16 - DNA, 26 bp, Artificial Sequence
  Chemically Synthesized
gcttaatacgactcactatagcccga

SEQ ID NO: 17 - DNA, 26 bp, Artificial Sequence
  Chemically Synthesized
gugtctctcttctgtcgggctatagt

SEQ ID NO: 18 - DNA, 50 bp, Artificial Sequence
  Chemically Synthesized
gtcgtatccagtgcagggtccgaggtattcgcactggatacgactcaaca

SEQ ID NO: 19 - DNA, 19 bp, Artificial Sequence
  Chemically Synthesized
cggcgtagcttatcagact

SEQ ID NO: 20 - DNA, 16 bp, Artificial Sequence
  Chemically Synthesized
gtgcagggtccgaggt

SEQ ID NO: 21 - DNA, 50 bp, Artificial Sequence
  Chemically Synthesized
gtcgtatccagtgcagggtccgaggtattcgcactggatacgaccactgg

SEQ ID NO: 22 - DNA, 18 bp, Artificial Sequence
  Chemically Synthesized
gacgtctcccaacccttg

SEQ ID NO: 23 - DNA, 16 bp, Artificial Sequence
  Chemically Synthesized
gtgcagggtccgaggt

SEQ ID NO: 24 - DNA, 50 bp, Artificial Sequence
  Chemically Synthesized
gtcgtatccagtgcagggtccgaggtattcgcactggatacgacaccact

SEQ ID NO: 25 - DNA, 19 bp, Artificial Sequence
  Chemically Synthesized
cggcgtagcttatcagact

SEQ ID NO: 26 - DNA, 16 bp, Artificial Sequence
  Chemically Synthesized
gtgcagggtccgaggt

SEQ ID NO: 27 - DNA, 20 bp, Artificial Sequence
  Chemically Synthesized
gctgtcctcctctgcaccat

SEQ ID NO: 28 - DNA, 20 bp, Artificial Sequence
  Chemically Synthesized
atctgccgggaggtgtagct

SEQ ID NO: 29 - DNA, 22 bp, Artificial Sequence
  Chemically Synthesized
catgctagtagctgccttctgc

SEQ ID NO: 30 - DNA, 20 bp, Artificial Sequence
  Chemically Synthesized
agcttcctcgcggtgtaaga

SEQ ID NO: 31 - DNA, 20 bp, Artificial Sequence
  Chemically Synthesized
gagcactgaaagcatgatcc

SEQ ID NO: 32 - DNA, 21 bp, Artificial Sequence
  Chemically Synthesized
cgagaagatgatctgactgcc

SEQ ID NO: 33 - DNA, 22 bp, Artificial Sequence
  Chemically Synthesized
agaagcgggtgagaaacaaaac

SEQ ID NO: 34 - DNA, 21 bp, Artificial Sequence
  Chemically Synthesized
agtgctgggtcaggaagtgtg

SEQ ID NO: 35 - DNA, 20 bp, Artificial Sequence
  Chemically Synthesized
caggctgctctgggattctc

SEQ ID NO: 36 - DNA, 21 bp, Artificial Sequence
  Chemically Synthesized
cctggaaggagcacttcatct

SEQ ID NO: 37 - DNA, 21 bp, Artificial Sequence
  Chemically Synthesized
gagtctgcccattgaggtcat

SEQ ID NO: 38 - DNA, 23 bp, Artificial Sequence
  Chemically Synthesized
aattttcatcctggatcagaacc

SEQ ID NO: 39 - DNA, 20 bp, Artificial Sequence
  Chemically Synthesized
cctttcccagagcatacagc

SEQ ID NO: 40 - DNA, 20 bp, Artificial Sequence
  Chemically Synthesized
ggacagaactcccacagagc

SEQ ID NO: 41 - DNA, 20 bp, Artificial Sequence
  Chemically Synthesized
cagagcatcaaccaaagcaa

SEQ ID NO: 42 - DNA, 20 bp, Artificial Sequence
  Chemically Synthesized
gctgccgtagcctcaaatac

SEQ ID NO: 43 - DNA, 24 bp, Artificial Sequence
  Chemically Synthesized
ctatcatcaacgggtacaaacgag

SEQ ID NO: 44 - DNA, 21 bp, Artificial Sequence
  Chemically Synthesized
cagatggatcagccaagaagg

SEQ ID NO: 45 - DNA, 22 bp, Artificial Sequence
  Chemically Synthesized
gtcttcaccaccatggagaagg

SEQ ID NO: 46 - DNA, 22 bp, Artificial Sequence
  Chemically Synthesized
atgatcttgaggctgttgtcat

SEQ ID NO: 47 - DNA, 21 bp, Artificial Sequence
  Chemically Synthesized
tcgcttcggcagcacatatac

SEQ ID NO: 48 - DNA, 21 bp, Artificial Sequence
  Chemically Synthesized
cgaatttgcgtgtcatccttg

SEQ ID NO: 49 - DNA, 29 bp, Artificial Sequence
  Chemically Synthesized
cagagtactaaccactatacgatcacggc

SEQ ID NO: 50 - DNA, 19 bp, Artificial Sequence
  Chemically Synthesized
gcgccgaatcctaaccact

SEQ ID NO: 51 - DNA, 21 bp, Artificial Sequence
  Chemically Synthesized
gccaatatttacgtgctgcta

SEQ ID NO: 52 - RNA, 23 nt, Artificial Sequence
  Chemically Synthesized
aaaccuaagaauaagcaagucau

SEQ ID NO: 53 - RNA, 21 nt, Artificial Sequence
  Chemically Synthesized
gccuugaggccaacaacauuu

SEQ ID NO: 54 - RNA, 21 nt, Artificial Sequence
  Chemically Synthesized
gguggugcuucaauuaauauu

SEQ ID NO: 55 - RNA, 20 nt, Artificial Sequence
  Chemically Synthesized
acuuucagguguuuccaaug

SEQ ID NO: 56 - RNA, 20 nt, Artificial Sequence
  Chemically Synthesized
uaggaaaccaucuagcccca

SEQ ID NO: 57 - RNA, 33 nt, Artificial Sequence
  Chemically Synthesized
guuuccguaguguagugguuaucacguucgccu

SEQ ID NO: 58 - RNA, 32 nt, Artificial Sequence
  Chemically Synthesized
gcauuggugguucagugguagaauucucgccu

SEQ ID NO: 59 - RNA, 36 nt, Artificial Sequence
  Chemically Synthesized
ucccugguggucuagugguuaggauucggcgcucuc

SEQ ID NO: 60 - RNA, 33 nt, Artificial Sequence
  Chemically Synthesized
guuuccguaguguaguggucaucacguucgccu

SEQ ID NO: 61 - RNA, 33 nt, Artificial Sequence
  Chemically Synthesized
ucccacauggucuagcgguuaggauuccugguu

SEQ ID NO: 62 - RNA, 33 nt, Artificial Sequence
  Chemically Synthesized
gcccggcuagcucagucgguagagcaugagacu

SEQ ID NO: 63 - RNA, 36 nt, Artificial Sequence
  Chemically Synthesized
uccucguuaguauaguggugaguauccccgccuguc

SEQ ID NO: 64 - RNA, 33 nt, Artificial Sequence
  Chemically Synthesized
agcagaguggcgcagcggaagcgugcugggccc

SEQ ID NO: 65 - RNA, 32 nt, Artificial Sequence
  Chemically Synthesized
ggcucguuggucuagggguaugauucucgcuu

SEQ ID NO: 66 - RNA, 35 nt, Artificial Sequence
  Chemically Synthesized
gucaggauggccgagcggucuaaggcgcugcguuc

SEQ ID NO: 67 - RNA, 33 nt, Artificial Sequence
  Chemically Synthesized
ggcuccguggcgcaauggauagcgcauuggacu

SEQ ID NO: 68 - RNA, 33 nt, Artificial Sequence
  Chemically Synthesized
gcccggauagcucagucgguagagcaucagacu

SEQ ID NO: 69 - RNA, 34 nt, Artificial Sequence
  Chemically Synthesized
gguuccauaguguagugguuaucacgucugcuuu

SEQ ID NO: 70 - RNA, 34 nt, Artificial Sequence
  Chemically Synthesized
gguuccaugguguaaugguuagcacucuggacuc

SEQ ID NO: 71 - RNA, 33 nt, Artificial Sequence
  Chemically Synthesized
gacccaguggccuaauggauaaggcaucagccu

SEQ ID NO: 72 - RNA, 33 nt, Artificial Sequence
  Chemically Synthesized
gggccaguggcgcaauggauaacgcgucugacu

SEQ ID NO: 73 - RNA, 37 nt, Artificial Sequence
  Chemically Synthesized
guuaagauggcagagccugguaauugcauaaaacuua

SEQ ID NO: 74 - RNA, 33 nt, Artificial Sequence
  Chemically Synthesized
gaccgcguggccuaauggauaaggcgucugacu

SEQ ID NO: 75 - RNA, 34 nt, Artificial Sequence
  Chemically Synthesized
gucucuguggcgcaaucgguuagcgcauucggcu

SEQ ID NO: 76 - RNA, 32 nt, Artificial Sequence
  Chemically Synthesized
ggggauguagcucagugguagagcgcgcgcuu

SEQ ID NO: 77 - RNA, 34 nt, Artificial Sequence
  Chemically Synthesized
gguagcguggccgagcggucuaaggcgcuggauu

SEQ ID NO: 78 - RNA, 32 nt, Artificial Sequence
  Chemically Synthesized
ggcuccauagcucagggguuagagcacugguc

SEQ ID NO: 79 - RNA, 33 nt, Artificial Sequence
  Chemically Synthesized
ggggaauuagcucaaaugguagagcgcucgcuu

SEQ ID NO: 80 - RNA, 33 nt, Artificial Sequence
  Chemically Synthesized
gucaggauggccgaguggucuaaggcgccagac

SEQ ID NO: 81 - RNA, 33 nt, Artificial Sequence
  Chemically Synthesized
gccgaaauagcucaguugggagagcguuagacu

SEQ ID NO: 82 - RNA, 33 nt, Artificial Sequence
  Chemically Synthesized
ggccccaugguguaaugguuagcacucuggacu

SEQ ID NO: 83 - RNA, 34 nt, Artificial Sequence
  Chemically Synthesized
ggccucguggcgcaacgguagcgcgucugacucc

SEQ ID NO: 84 - RNA, 36 nt, Artificial Sequence
  Chemically Synthesized
gacgagguggccgagugguuaaggcgauggacugcu

SEQ ID NO: 85 - RNA, 33 nt, Artificial Sequence
  Chemically Synthesized
ggggguauagcucaguggguagagcauuugacu

SEQ ID NO: 86 - RNA, 35 nt, Artificial Sequence
  Chemically Synthesized
ggccgguuagcucagucggcuagagcguggugcua

SEQ ID NO: 87 - RNA, 33 nt, Artificial Sequence
  Chemically Synthesized
gccccaguggccuaauggauaaggcauuggccu

SEQ ID NO: 88 - RNA, 32 nt, Artificial Sequence
  Chemically Synthesized
guagucguggccgagugguuaaggcgauggac

SEQ ID NO: 89 - RNA, 33 nt, Artificial Sequence
  Chemically Synthesized
gcguuggugguauagugguuagcauagcugccu

SEQ ID NO: 90 - RNA, 34 nt, Artificial Sequence
  Chemically Synthesized
gguaguguggccgagcggucuaaggcgcuggauu

SEQ ID NO: 91 - RNA, 32 nt, Artificial Sequence
  Chemically Synthesized
ggggauguagcucagugguagagcgcaugcuu

SEQ ID NO: 92 - RNA, 33 nt, Artificial Sequence
  Chemically Synthesized
ggcagaguggugcagcggaagcgugcugggccc

SEQ ID NO: 93 - RNA, 20 nt, Artificial Sequence
  Chemically Synthesized
gcccgacagaagagagacuc